(19) United States
(12) Patent Application Publication
(10) Pub. No.: US 2026/0120865 A1
(43) Pub. Date: Apr. 30, 2026

(54) Systems and Methods to Assess Neonatal Health Risk and Uses Thereof

(71) Applicant: The Board of Trustees of the Leland Stanford Junior University, Stanford, CA (US)

(72) Inventors: Nima Aghaeepour, Mountain View, CA (US); Davide De Francesco, Stanford, CA (US); Jonathan Reiss, Stanford, CA (US); David K. Stevenson, Stanford, CA (US)

(73) Assignee: The Board of Trustees of the Leland Stanford Junior University, Stanford, CA (US)

(21) Appl. No.: 18/840,454

(22) PCT Filed: Feb. 28, 2023
(86) PCT No.: PCT/US2023/014186

§ 371 Date: Aug. 21, 2024

Related U.S. Application Data
(60) Provisional application No. 63/268,689, filed on Feb. 28, 2022.

(51) Int. Cl. G16H 50/20; G16H 10/60; G16H 20/40; G16H 20/60

(52) U.S. Cl. CPC G16H 50/20; G16H 10/60; G16H 20/40; G16H 20/60

(57) ABSTRACT

Embodiments herein describe systems and methods to assess neonatal health risk and uses thereof. Many embodiments utilize one or more machine learning models to identify neonatal disorders based on electronic health records (EHR) for an individual. Further embodiments treat, remediate, ameliorate, or mitigate the disorder.

### CROSS-REFERENCE TO RELATED APPLICATIONS

The current application claims priority to U.S. Provisional Patent Application No. 63/268,689, entitled “Systems and Methods to Assess Neonatal Health Risk and Uses Thereof” to Aghaeepour et al., filed Feb. 28, 2022, the disclosure of which is hereby incorporated by reference in its entirety.

### STATEMENT REGARDING FEDERALLY SPONSORED RESEARCH OR DEVELOPMENT

This invention was made with Government support under contracts HL139844 and GM138353 awarded by the National Institutes of Health. The Government has certain rights in the invention.

### FIELD OF THE INVENTION

The present invention relates to neonatal health; more specifically, the present invention relates to systems and methods incorporating machine learning for identifying neonatal risk, especially in preterm births.

### BACKGROUND

Prematurity is the leading cause of death in children under 5 years of age. Although gestational age and birth weight along with other anthropometric indices give clinicians a crude approximation of risk for neonatal morbidities and mortality, these data are increasingly recognized as poor surrogates. For example, while gestational age is commonly viewed as a surrogate for biologic immaturity, this variable alone performs poorly as a risk predictor. For instance, many infants born before 28 weeks' gestation develop at least one of the sequelae of prematurity including intraventricular hemorrhage (IVH), respiratory distress syndrome (RDS), necrotizing enterocolitis (NEC), sepsis (early or late), retinopathy of prematurity (ROP), BPD and periventricular leukomalacia (PVL).6 Some preterm neonates develop more than 1-2 of these entities; rarely do babies have none of them.

Accurate risk prediction and prognostication is crucial in perinatal and neonatal medicine. Validated clinical prediction calculators have estimated risk trajectories for common outcomes related to prematurity, including death, neurodevelopmental impairment, bronchopulmonary dysplasia (BPD) and others. Prognostic estimates help clinicians and families choose reasonable interventions to pursue in hopes of securing the outcomes(s) they value or most desire. Historically, pre- and post-natal risk calculators have incorporated a small set of clinical risk factors assessed at single time point, giving families and providers an approximate estimate of risk for their fetus or newborn. To date, most clinical prediction calculators have limited predictive power and clinical utility owing to the small number of parameters considered and the single time point utilized.

Understanding which premature neonates are more likely to develop an acquired complication of prematurity based on their underlying level of personal risk, is a critical quest aligned with the precision medicine mandate of the 21st century.

### SUMMARY OF THE INVENTION

This summary is meant to provide some examples and is not intended to be limiting of the scope of the invention in any way. For example, any feature included in an example of this summary is not required by the claims, unless the claims explicitly recite the features. Various features and steps as described elsewhere in this disclosure may be included in the examples summarized here, and the features and steps described here and elsewhere can be combined in a variety of ways.

In some aspects, the techniques described herein relate to a machine learning model, including a multitask neural network, where the neural network includes an encoder, a hidden state, and a decoder, where the encoder reads an input, where the hidden state represents an internal learned representation of the entire input, and where the decoder interprets the interprets internal learned representation and reconstructs the input.

In some aspects, the techniques described herein relate to a machine learning model, where the input includes electronic health records (EHR) for an individual.

In some aspects, the techniques described herein relate to a machine learning model, where the machine learning model is trained using EHR for a plurality of individuals and a plurality of newborns, where each individual in the plurality of individuals has birthed at least one newborn in the plurality of newborns.

In some aspects, the techniques described herein relate to a method for assessing neonatal risk, including obtaining or having obtained electronic health records (EHR) for an individual, and identifying at least one neonatal disorder for a child of the individual based on the metabolites in the EHR utilizing a machine learning model including deep learning neural network with at least one bottleneck layer.

In some aspects, the techniques described herein relate to a method, where the machine learning model determines respiratory support strategies (including ventilator settings) to reduce adverse outcomes.

In some aspects, the techniques described herein relate to a method, where the respiratory support strategies includes ventilator settings.

In some aspects, the techniques described herein relate to a method, where the model identifies a medication prescribed to the mother than can impact neonatal morbidities.

In some aspects, the techniques described herein relate to a method, where the at least one neonatal disorder is selected from bronchopulmonary dysplasia (BPD), intraventricular hemorrhage (IVH), necrotizing enterocolitis (NEC), retinopathy of prematurity (ROP), Bronchopulmonary dysplasia (BPD), intraventricular hemorrhage (IVH), necrotizing enterocolitis (NEC), retinopathy of prematurity (ROP), pulmonary hypertension, pulmonary hemorrhage, jaundice, periventricular leukomalacia (PVL), respiratory distress syndrome (RDS), early onset sepsis, late onset sepsis, patent ductus arteriosus (PDA), cerebral palsy, and neurodevelopmental impairment (NDI).

In some aspects, the techniques described herein relate to a method, where the EHR comes from multiple institutions.

In some aspects, the techniques described herein relate to a method, further including treating the child for the at least one neonatal disorder.

In some aspects, the techniques described herein relate to a method for providing intravenous nutrients to a premature baby, including obtaining or having obtained electronic health records (EHR) for an individual, where the EHR include details about the individual's health, and the individual is a premature baby, and selecting a nutrient bag including a mix of nutrients to supplement the health of the individual.

In some aspects, the techniques described herein relate to a method, where the machine learning model includes a multitask neural network, where the neural network includes an encoder, a hidden state, and a decoder, where the encoder reads an input, where the hidden state represents an internal learned representation of the entire input, and where the decoder interprets the interprets internal learned representation and reconstructs the input.

In some aspects, the techniques described herein relate to a method, where the nutrient bag is one bag of a set of nutrient bags, where each bag in the set of nutrient bags is included of a composition of nutrients generated by clustering from a bottleneck layer.

In some aspects, the techniques described herein relate to a method, where the nutrient bag improves wound healing.

In some aspects, the techniques described herein relate to a method, where the nutrient bag improves neurocognitive development.

In some aspects, the techniques described herein relate to a method, where the nutrient bag improves respiratory health.

In some aspects, the techniques described herein relate to a method, where the nutrient bag improves gastrointestinal health.

In some aspects, the techniques described herein relate to a method, where the nutrient bag improves eye health.

In some aspects, the techniques described herein relate to a method for nutritional support, including obtaining health information about an individual, and providing a dietary recommendation for the individual.

In some aspects, the techniques described herein relate to a method, where providing a dietary recommendation includes providing a food recommendation.

In some aspects, the techniques described herein relate to a method, where the food recommendation includes at least one baby food recommendation.

In some aspects, the techniques described herein relate to a method, where providing a dietary recommendation includes interfacing with a database of foods and nutritional information.

In some aspects, the techniques described herein relate to a method for manufacturing intravenous nutritional supplement solutions, including developing a set of nutritional recipes for intravenous supplementation using a machine learning model, producing a nutrient bag including a recipe from the set of recipes.

In some aspects, the techniques described herein relate to a method, where the machine learning model includes a multitask neural network, where the neural network includes an encoder, a hidden state, and a decoder, where the encoder reads an input, where the hidden state represents an internal learned representation of the entire input, and where the decoder interprets the interprets internal learned representation and reconstructs the input.

In some aspects, the techniques described herein relate to a method, where producing a nutrient bag includes producing a nutrient bag for each recipe in the set of recipes.

In some aspects, the techniques described herein relate to a method, where the set of nutritional recipes includes at least 5 recipes.

In some aspects, the techniques described herein relate to a method, where the set of nutritional recipes includes 15 recipes.

In some aspects, the techniques described herein relate to a method, where the nutrient bag is a sterile IV bag.

Other features and advantages of the present invention will become apparent from the following detailed description, taken in conjunction with the accompanying drawings which illustrate, by way of example, the principles of the invention.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates an exemplary LSTM-based autoencoder that enables objective identification of subgroups with enhanced Performance for an AI model in accordance with various embodiments. Specifically, subgroup discovery performed on the feature latent space obtained using a LSTM autoencoder identified subgroups of newborns where the AI model has high precision-recall. FIG. 1 provides an exemplary architecture of the exemplary LSTM-based autoencoder used to extract a lower-dimensional encoded representation of the input sequences containing the maternal EHR history. Subgroup discovery proceeds iteratively at each level by dividing the dataset into many overlapping subgroups defined by variables of the obtained latent space. The search path for a single subgroup proceeds down two levels. At the end of the procedure, subgroups are scored and ranked based on predefined scoring criteria (i.e. AUPRC) for further analysis. Classification accuracy, in terms of AUC, AUPRC and AUPRC compared to a random classifier, in subgroups identified through subgroup discovery and in the full dataset.

FIG. 2 illustrates an exemplary correlation plot of EHR codes in maternal medical histories and measurements in accordance with various embodiments, where each node represents a code or measurement. The size of the node is proportional to the metric described in the methods to assess feature importance, averaged across all outcomes; edges connect nodes whose correlation is among the top 1% of all correlations.

FIG. 3 illustrates an exemplary AUC of an AI model in pre-term newborns (born <37 weeks of gestation) and full-term newborns (born ≥37 weeks of gestation) for the different neonatal outcomes in accordance with various embodiments. None of the full-term newborns had PVL or anemia, these outcomes were therefore excluded from the plot.

FIGS. 4A-4D illustrate an exemplary overview of an AI pipeline for prediction of neonatal outcomes in accordance with various embodiments. FIG. 4A provides an example of a hypothetical patient timeline with multiple visits before and after delivery/birth; at each visit, any combination of conditions, observations, medications and procedures can be recorded. FIG. 4B provides an exemplary architecture of the multi-input multi-task deep learning model: the sequence of codes from the maternal/newborn medical history, after code embeddings, is fed into a bi-directional LSTM layer with 128 units, while maternal/newborn socio-demographic information, maternal measurements and, when specified, gestational age and birthweight are fed into a 4-unit dense layer. The outputs of these two networks are then concatenated and fed into a dense one-layer neural network with 64 units followed by a set of dense layers, one set for each outcome, consisting of two dense layers and a single-unit output. FIG. 4C provides an exemplary bi-directional LSTM layer to learn bidirectional long-term dependencies between codes within a sequence: each code in the sequence is fed into a forward and a backward LSTM layer and the outputs of the two layers are further concatenated. While processing, the hidden state from the layer of the previous code in the sequence is passed to the layer of the following code of the sequence; the hidden state acts as the memory of the neural network, holding information on previous data the network has seen before. FIG. 4D provides an exemplary structure of a single LSTM layer for the t-th code in the sequence: ct is the cell state that carries relevant information throughout the processing of the sequence, ht is the hidden state that of the t-th code in the sequence that is passed to the layer of the next code in the sequence, xt is the input to the layer processing the t-th code in the sequence, i.e. the vector corresponding to the embeddings of the t-th code in the sequence. Each line carries an entire vector, circles represent pointwise operations, boxes represent learned neural network layers with the indicated activation function. Lines merging denote concatenation, line forking denotes the content is copied and the copies going to different locations. Basically, the LSTM layer learns what information has to be discarded from the cell state and what new information has to be stored in the cell state; finally, the output is calculated based on the cell state and the processed input.

FIGS. 5A-5D provide exemplary data showing proportions of codes in various situations. Specifically, FIG. 5A shows the proportion of codes from each feature set out of the total number of unique codes found in medical histories of the 32,354 newborns and the 27,519 mothers; FIG. 5B shows the proportion of codes from each feature set out of all the codes found in medical histories; FIG. 5C shows an average percentage decrease in AUC across the 24 outcomes for the AI model not including a specific feature set; FIG. 5D shows an average percentage decrease in AUPRC across the 24 outcomes for the AI model not including a specific feature set.

FIG. 6 illustrates exemplary data showing the proportion of maternal medical histories in which each concept code was present up to delivery in Stanford (x-axis) and UCSF (y-axis) pregnancies; axes are in the logit scale; the black solid line indicates perfect agreement (i.e. the % is the same in UCSF and Stanford); black dashed lines indicates when the % in one dataset was between half and twice the % in the other dataset; concept codes outside of these dashed line were excluded from subsequent analyses.

FIGS. 7A-7B illustrate exemplary data showing multitask analysis of EHR data between 2014-2020 results in a longitudinal and comprehensive predictive model of neonatal morbidity before and after birth. Prediction AUC at birth is >0.7 for 22/24 outcomes, >0.8 for 17/24, and >0.9 for 10/24. Neonatal outcomes with AUC>0.9 at birth include BPD, ROP, Anemia of Prematurity, Death, IVH, Cardiac Failure, PVL, Pulm hem, NEC, and Atelectasis. AUC is >0.9 for 21/24 at 1W postnatal age and including outcomes with long latency periods prior to diagnosis such as BPD, ROP and NEC. Prediction of neonatal morbidities in real-time can be difficult to ascertain, especially for less common outcomes such as IVH, NEC, Sepsis and Death. The heat map is shaded according to the multitask modeling output that results from a 1) specific time period and 2) encompasses the associated clinical inputs (medications, measurements, conditions, observations and procedures) that result in (FIG. 7A) Fold increase/decrease in AUPRC of the AI model compared to a random classifier at different timepoints, from 5 months before delivery/birth (−5M) up to two months after delivery/birth (+2M), 0 indicated delivery/birth or (FIG. 7B) AUC for an individual outcome at different time points. All outcomes prior to birth incorporate maternal codes at either 5M, 4M, 3M, 2M, 1M, 2W, or 1W before birth. All outcomes at birth incorporate all maternal inputs up to and including delivery. All outcomes after birth incorporate maternal and neonatal inputs up to a specific postnatal time point (e.g. 1 week, 2 weeks, 1 month or 2 months). The linkage between maternal (n=27,519) and neonatal (n=32,354) clinical data has allowed us to build generalizable longitudinal models that inform risk prediction for all neonatal outcomes. Neonatal outcomes on the y-axis are arranged based on cluster analysis. Comprehensive longitudinal calculators that incorporate maternal, fetal and neonatal risk factors have the potential to transform clinical care through standardization of risk assessment, early identification of high-risk populations that may benefit from additional therapies and/or enrollment in clinical trials, and timely intervention prior to the development of an outcome.

FIG. 8 illustrates exemplary data showing a tetrachoric correlation plot of the 24 neonatal outcomes considered: the size of the node is proportional to the prevalence in the study dataset; nodes are connected if the correlation is greater than 0.5, thickness and the color of the edges are proportional to the strength of the correlation, with darker green color and thicker lines showing stronger correlations; outcomes of prematurity including RDS, IVH, Sepsis, NEC, ROP and Anemia of Prematurity are shown to be highly correlated.

FIG. 9 illustrates an exemplary hypothetical prediction timeline for a newborn with BPD; the predicted score from the AI model at different timepoints is based on various risk factors obtained from EHR records in the maternal and newborn history. Throughout pregnancy, at birth, and in the postnatal period, additional data is incorporated into the model and the prediction model iteratively improves. Patients with higher BPD prediction scores (i.e. 0.2), have a greater propensity to develop BPD (twice as likely) compared to those with lower BPD prediction scores (i.e. 0.1) and can be directly compared as such. BPD prediction scores should not be interpreted as individual probabilities for the later development of BPD.

FIGS. 10A-10B illustrate exemplary data of AUC of an AI model for the prediction of the 24 neonatal outcomes at different timepoints, from 5 months before delivery/birth (−5M) up to 2 months after delivery/birth (+2M); the vertical dashed line indicates delivery/birth; the shaded area indicates the 95% confidence interval for the AUC; the horizontal dotted line indicates the AUC of a random classifier (i.e. 0.5).

FIGS. 10C-10D illustrate exemplary data of AUPRC of an AI model for the prediction of the 24 neonatal outcomes at different timepoints, from 5 months before delivery/birth (−5M) up to 2 months after delivery/birth (+2M); the vertical dashed line indicates delivery/birth; the shaded area indicates the 95% confidence interval for the AUPRC; the horizontal dotted line indicated the AUPRC of a random classifier, equivalent to the prevalence of the outcome in the dataset.

FIG. 11 illustrates an example of neonatal outcome prediction scores for an individual dichorionic-patient born at Lucile Packard Children's Hospital on Apr. 13, 2020 at gestational age of 24 weeks, 2 days following PPROM, chorioamnionitis, and spontaneous PTL. Risk prediction is calculated based on maternal and neonatal codes that chronologically lead up to and include a specific diagnosis but do not extend beyond the date of an individual diagnosis (when this occurs). The patient ultimately had EHR diagnoses of RDS, IVH (Grade I bilateral), BPD, Sepsis, PDA, Anemia of Prematurity, ROP and Hyperbilirubinemia. The individual prediction score at birth was highest for ROP, Anemia of Prematurity, R D S and Hyperbilirubinemia, all diagnoses for which the patient ultimately had. The prediction score at birth was lowest for NEC, Pulmonary Hypertension, CP, PVL, and Death. Despite this infant's high risk for these diagnoses the patient is alive and never developed any of these outcomes with the exception of transient Pulmonary Hypertension. This model allows us to predict individual outcomes based on population-level data. We acknowledge and thank the parents of this patient who gave us permission to create and publish this individuals' risk prediction score.

FIGS. 12A-12B illustrate an exemplary time-shifting experiment dataset. AUC of the AI model for the prediction of the 24 neonatal outcomes at delivery/birth in the whole dataset (tested using 5-fold cross-validation, black line), in newborns born in 2019 only (n=5,852, light green line), and in newborns born in 2020 only (n=4,397, dark green line).

FIGS. 12C-12D illustrate an exemplary time-shifting experiment dataset. AUPRC of the AI model for the prediction of the 24 neonatal outcomes at delivery/birth in the whole dataset (tested using 5-fold cross-validation, black line), in newborns born in 2019 only (n=5,852, light green line), and in newborns born in 2020 only (n=4,397, dark green line).

FIG. 13A illustrates exemplary AUPRC of simplified models to predict the five selected outcomes in the train dataset (Stanford) and in the external validation data (UCSF).

FIG. 13B illustrates exemplary AUC of simplified models to predict the five selected outcomes in the train dataset (Stanford) and in the external validation data (UCSF).

FIGS. 14A-14B illustrate exemplary AUC at delivery/birth of the AI model (black solid line) and of the APGAR at 1 minute (orange dashed line); the grey dashed line indicates the AUC of a random classifier. The APGAR score at 1 minute is composed of 5 discrete subjective scores (each scored 0-2) composed of 1) appearance 2) heart rate 3) grimace 4) activity and 5) respiratory effort. The AI model has similar AUC's to the APGAR model for outcomes such as RDS, Death, Sepsis, Pulmonary Hemorrhage, and Other CNS of which hypoxic ischemic encephalopathy (mild, moderate and severe) are included. Of note, the APGAR score is a subjective group of physical exam findings obtained shortly after birth and does not have adequate predictive capabilities for any known neonatal outcome.

FIGS. 14C-14D illustrate exemplary AUPRC at delivery/birth of the AI model (black solid line) and of the APGAR at 1 minute (orange dashed line).

FIG. 15A illustrates an exemplary odds ratio between condition concept codes (row) and neonatal outcomes (columns); the 50 condition concept codes with the highest average odds ratio across outcomes are displayed; the color indicates the strength and direction of the association with red indicating a positive association (i.e. the presence of the concept code in the maternal EHR history was associated with an increased risk of the outcome in the newborn) and blue indicating a negative association (i.e. i.e. the presence of the concept code in the maternal EHR history was associated with a decreased risk of the outcome in the newborn).

FIG. 15B illustrates an exemplary odds ratio between observation concept codes (row) and neonatal outcomes (columns); the 50 observation concept codes with the highest average odds ratio across outcomes are displayed; the color indicates the strength and direction of the association with red indicating a positive association (i.e. the presence of the concept code in the maternal EHR history was associated with an increased risk of the outcome in the newborn) and blue indicating a negative association (i.e. i.e. the presence of the concept code in the maternal EHR history was associated with a decreased risk of the outcome in the newborn).

FIG. 15C illustrates an exemplary odds ratio between medication concept codes (row) and neonatal outcomes (columns); the 50 medication codes with the highest average odds ratio across outcomes are displayed; the color indicates the strength and direction of the association with red indicating a positive association (i.e. the presence of the concept code in the maternal EHR history was associated with an increased risk of the outcome in the newborn) and blue indicating a negative association (i.e. i.e. the presence of the concept code in the maternal EHR history was associated with a decreased risk of the outcome in the newborn). Of note, there is inherent indication bias for medications commonly used in the setting of preterm delivery such as magnesium sulfate, indomethacin and 17-alphaa-hydroxyprogesterone. In addition medications to prevent hypertensive conditions of pregnancy (nifedipine, aspirin), or various forms of diabetes (insulin, glyburide, glucagon,) all demonstrated positive associations as would be expected.

FIG. 15D illustrates an exemplary odds ratio between procedure concept codes (row) and neonatal outcomes (columns); the 50 procedure concept codes with the highest average odds ratio across outcomes are displayed; the color indicates the strength and direction of the association with red indicating a positive association (i.e. the presence of the concept code in the maternal EHR history was associated with an increased risk of the outcome in the newborn) and blue indicating a negative association (i.e. i.e. the presence of the concept code in the maternal EHR history was associated with a decreased risk of the outcome in the newborn).

FIG. 15E illustrates an exemplary odds ratio between the last measurement value recorded one week before delivery/birth (row) and neonatal outcomes (columns); the 50 measurements with the highest average odds ratio across outcomes are displayed; the color indicates the strength and direction of the association with red indicating a positive association (i.e. a value above the median was associated with an increased risk of the outcome in the newborn) and blue indicating a negative association (i.e. a value below the median was associated with a decreased risk of the outcome in the newborn). Of note, the protective effect of higher albumin levels may reflect superior nutrition associated with many of the neonatal outcomes.

FIG. 16 illustrates an exemplary correlation network of the top 20 conditions, medications, observations, procedures and measurements with the strongest association across all the 24 neonatal outcomes; the metric obtained from odds ratios as described in the methods was used to rank 20 conditions, medications, measurements, procedures and measurements and select the top 20 within each set with the highest average across neonatal outcomes. A tSNE map of the resulting features was constructed; nodes represent conditions, observations, procedures, medications and measurements; edges connect nodes with a correlation exceeding 0.8 (red edges represent negative correlations, green edges represent positive correlations; correlation was assessed using tetrachoric, biserial, or Pearson's correlation coefficient, as appropriate; the size of the nodes is proportional to the average odds ratio across the 24 outcomes, the larger the node the stronger is the average association across outcomes.

FIGS. 17A-17B illustrate exemplary AUC of the multi-task AI model (in dark blue), simultaneously predicting the 24 neonatal outcomes, and the separate single-task models (in light blue) each predicting one individual outcome; the grey dashed line indicates the AUC of a random classifier.

FIGS. 17C-17D illustrate exemplary AUPRC of the multi-task AI model (in dark blue), simultaneously predicting the 24 neonatal outcomes, and the separate single-task models (in light blue) each predicting one individual outcome.

FIGS. 18A-18F illustrate exemplary data of pathological mechanisms underlying NEC that are leveraged by the multi-task approach to improve NEC predictions. Specifically FIG. 18A illustrates a correlation network of the top 20 conditions, medications, observations, procedures and measurements with the strongest association across all the 24 neonatal outcomes including NEC; the metric obtained from odds ratios as described in the methods was used to rank conditions, medications, measurements, procedures and measurements and select the top 20 within each set with the highest average across neonatal outcomes. A tSNE map of the resulting features was constructed; nodes represent conditions, observations, procedures, medications and measurements; edges connect nodes with a correlation exceeding 0.8; correlation was assessed using tetrachoric, biserial, or Pearson's correlation coefficient, as appropriate; the size of the nodes is proportional to the odds ratio of NEC. The larger the node the stronger is the association with the outcome, regardless of the direction, i.e. positive or negative association. FIG. 18B illustrates exemplary AUC for the prediction of NEC of the single-task model (black dashed line), the two-output multi-task model simultaneously predicting NEC and polycythemia (green line), and the two-output multi-task model simultaneously predicting NEC and anemia of prematurity (blue line). FIG. 18C illustrates an exemplary comparison of maternal hemoglobin levels for infants diagnosed with NEC compared to those not diagnosed with NEC. Statistically significant differences in maternal hemoglobin levels occurred at 5M, 4M, and 1M prior to delivery. FIG. 18D illustrates an exemplary comparison of neonatal hemoglobin levels at birth, 1M, 2M, 3M and 4M of age for neonates diagnosed with NEC versus those not diagnosed with NEC. Infants who developed NEC had lower hemoglobin concentrations at birth compared to infants who did not develop NEC. FIG. 18E illustrates exemplary data of maternal hemoglobin level at time of delivery versus NEC predicted score for neonates diagnosed with NEC and those never diagnosed with NEC. FIG. 18F illustrates exemplary data of newborn hemoglobin level at birth versus NEC predicted score for neonates diagnosed with NEC and those never diagnosed with NEC.

FIGS. 19A-19B illustrate exemplary data showing that an AI model is able to distinguish according to IVH grading and to provide insight into the pathological mechanisms underlying IVH. FIG. 19A provides an exemplary correlation network of the top 20 conditions, medications, observations, procedures and measurements with the strongest association across all the 24 neonatal outcomes; the metric obtained from odds ratios as described in the methods was used to rank conditions, medications, measurements, procedures and measurements and select the top 20 within each set with the highest average across neonatal outcomes. A tSNE map of the resulting features was constructed; nodes represent conditions, observations, procedures, medications and measurements; edges connect nodes with a correlation exceeding 0.8; correlation was assessed using tetrachoric, biserial, or Pearson's correlation coefficient, as appropriate; the size of the nodes is proportional to the odds ratio with IVH, the larger the node the stronger is the association with the outcome, regardless of the direction, i.e. positive or negative association. FIG. 19B provides exemplary IVH predicted scores from the AI model at delivery in newborns stratified by IVH grading. The predictive power of the model increases in a stepwise manner with the IVH clinical severity such that Grade IV>Grade III>Grade II>Grade I. Patients with higher IVH prediction scores (i.e. 0.2), have a greater propensity to develop IVH (twice as likely) compared to those with lower IVH prediction scores (i.e. 0.1) and can be directly compared as such. IVH prediction scores should not be interpreted as individual probabilities for the development of IVH. Neonates in the unspecific grade category had discrepancies in the IVH Grade reported in the ultrasound reports and their ICD coding such that it was difficulty to classify them according to the Papile grading system.

### DETAILED DESCRIPTION

Turning now to the drawings, systems and methods to assess neonatal health risk and uses thereof are provided. Many embodiments provide methods that include a machine learning model to improve risk prediction by integrating serial and rich neonatal and maternal information contained in electronic health records (EHR) collected before and after birth. Further embodiments describe methods that predict a likelihood of one or more disorders to which preterm babies are susceptible. Certain embodiments describe recommendations to treat, remediate, ameliorate, or mitigate one or more disorders that are predicted. Further embodiments allow for risk stratification of a preterm baby based on the likelihood of that baby having any disorder.

Over the last decade, hospital systems have increasingly implemented EHR systems to capture and store clinical data in real time. Longitudinal data capture along and the serialization of clinical information for patients with both acute and chronic health conditions, inpatient hospital stays, and outpatient care have revolutionized clinical medicine. EHRs have allowed formalized communication of large amounts of data among providers and has streamlined billing, and to some extent, research workflows. However, EHR clinical data are notoriously complex, and difficult to interrogate. They are also heterogenous and lack standardization. Recent computational advances help mitigate such limitations by data linkage and the availability of vast amounts of demographic, diagnostic, medication and clinical data. Moreover, these data can often be retrieved at a fraction of the time and cost spent on prospective cohort studies or clinical trials and include thousands or tens of thousands of additional patients.

From an analytical point of view, EHR data present challenges that traditional computational approaches fail to address. These include incorporation of longitudinal information with temporal dependencies, and modeling thousands of potential predictors, arising from the complexity and granularity of the data. Recent developments in artificial intelligence (AI) methods allow addressing many of these challenges thereby fully leveraging the breadth of EHR data. AI models, such as artificial neural networks can handle large volumes of structured and unstructured data with large numbers of input variables. In particular, recurrent neural networks such as long short-term memory (LSTM) models, are designed to utilize temporal dependencies and do not need to specify a priori which potential predictor variables should be considered. Moreover, multi-task learning allows us to predict multiple outcomes simultaneously. By leveraging underlying commonalities among outcomes, the knowledge learned in predicting one outcome is shared when predicting other outcomes, thus improving predictive power when compared to models developed to predict each outcome independently.

Many embodiments herein leverage multi-task learning to simultaneously predict the risk for the most important adverse neonatal outcomes using longitudinal EHR data spanning a period starting shortly after the time of conception, and ending months after birth.

### Machine Learning Models

Artificial neural networks (NNs) are a family of computing systems based on a collection of connected units or nodes, which receive a signal (input data or the signal returned by previous units), process it and then transmit it to the following units. Units are aggregated into layers, and each layer may perform different transformations on their inputs. Signals travel from the first layers (the input layers), to the last layers (the output layers containing the object of the prediction). Many embodiments use NNs due their ability to process vast amount of data, to learn and model complex non-linear relationships that can be generalized to unseen data, and because NNs do not require strict assumptions regarding the distribution of input variables and their associations. In the presence of multiple outcomes, multi-task learning allows prediction of multiple outcomes at the same time by leveraging representations that are shared across related outcomes. Additionally, recurrent NNs (RNNs) use their internal state (e.g., memory), taking information from prior inputs to influence the current input and output. Unlike traditional NNs, where inputs and outputs are independent of each other, the output of recurrent NNs depends on the prior elements within the sequence. Long Short-Term Memory (LSTM) are a particular type of RNN, proposed to address the problem of long-term dependencies, e.g., when the previous state that is influencing the current prediction is not in the recent past, but in a more distant past.

In many embodiments, LSTM RNNs are characterized by “cells” in the hidden layers of the NN, which have three gates (e.g., an input gate, an output gate, and a forget gate). These gates control the flow of information allowing the LSTM layer to remember the information for longer periods. In regular (uni-directional) LSTM NNs the input flows in one direction, typically forward, i.e. from past to future. In bi-directional LSTM NNs the input flows in both directions to preserve both future and past information.

Many embodiments use one or more multi-input multi-task deep neural networks for the prediction of neonatal outcomes, determine nutritional needs, and/or any other use described herein. Some embodiments utilize one or more multi-input multi-task deep neural networks to identify and/or discover subgroups within a population. In numerous embodiments, the one or more multi-input multi-task deep neural networks includes an autoencoder. Autoencoders are a self-supervised learning model that can learn a compressed, lower dimensional representation of the input data. An autoencoder typically consists of an encoder and a decoder: the encoder reads an input sequence; the hidden state or output of an encoder represents an internal learned representation of the entire input sequence that is then provided as an input to a decoder model, which interprets the internal learned representation and reconstructs the input sequence. Additionally, subgroup discovery is a data mining technique that identifies descriptions of data subsets showing an interesting distribution with respect to a pre-specified target. For example: given a dataset X and a search space S identified by a set of descriptors (i.e. variables), subgroup discovery finds and ranks subgroups of X where a target concept is high or low.

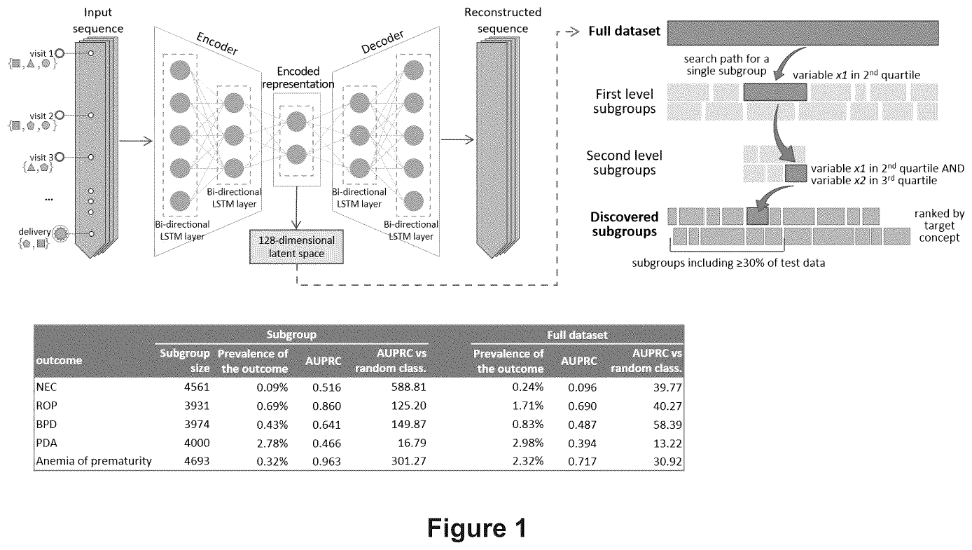

Turning to FIG. 1, in one exemplary embodiment, the input of the autoencoder consists of a sequences of concept codes. These codes can be fed into an appropriate layer or layers of an encoder. In various embodiments, the layers are LSTM layers, convolutional layers, and/or any appropriate layer time. In the illustrated example, the layers include a 256-unit bi-directional LSTM layer followed by a 128-unit bi-directional LSTM layer. The output of the second layer is an encoded 128-dimensional latent space of the input data that can be used to identify subgroups using subgroup discovery. A bridging layer can be used to connect an encoder and decoder. As further illustrated in this example, the bridging layer is a repeat vector layer, but any appropriate layer can be used within embodiments. The decoder can take any number of layers and/or layer types to reconstruct an input sequence. In some embodiments, such as illustrated in FIG. 1, the decoder can consist of two bi-directional LSTM layers that mirror the two layers of encoder—e.g., a 128-unit bidirectional LSTM layer followed by a 256-unit bidirectional LSTM layer.

### Training Machine Learning Model

Many embodiments train a model based on input derived from EHRs including (but not limited to) conditions, observations, medications, procedures and measurements recorded under a mother's patient identification number. Exemplary measurements can include test results that indicate one or more of an individual's genetics, blood panel results, enzymes, metabolites, fatty acids, and/or any other measurable component. In various embodiments, the records include: 1) Conditions: presence of a disease or medical condition, 2) Observations: observed clinical sequelae obtained as part of the medical history, 3) Medications: utilization of any prescribed and over-the-counter medicines, vaccines, and large-molecule biologic therapies, 4) Procedures: records of activities or processes ordered by or carried out by a healthcare provider on the patient for a diagnostic or therapeutic purpose, and 5) Measurements: structured values obtained through systematic and standardized examination or testing of a patient or patient's sample such as laboratory tests, vital signs, quantitative findings from pathology reports, etc., Conditions, observations, medications and procedures are organized by patient and time, and records corresponding to conditions used to identify newborn's outcomes are excluded to avoid potential leakage of information about the outcomes into the input data. The resulting entire sequence of time-ordered records, up to the timepoint of prediction (e.g. delivery, one week before delivery, two weeks before delivery, etc.), formed one of the newborn's personalized input to the model. In additional embodiments, the most common measurements (e.g., available in ≥10% of mothers) are extracted to form an additional newborn's personalized input together with maternal demographics (age at delivery and ethnicity), and, when specified, newborn's sex, gestational age at delivery, and birthweight. Table 1 provides a list of measurements, which can be used in various embodiments.

Additionally, many embodiments further include the entire medical histories for each newborn, including all conditions, observations, medications, and procedures—these events are extracted and organized by time. For each neonate, the resulting sequence of records are combined to the sequence of records of the respective mother (up to delivery/birth) to form the input data for models at points of prediction after delivery. Certain embodiments can further use inputs derived from medications, nutritional supplements, diets, and/or any other relevant aspect that can play a role in health and/or development.

To predict a range of neonatal outcomes and to fully interrogate the shared neonatal pathologies via the multi-task approach, a list of neonatal outcomes can be obtained as the presence or absence of any record related to each of these outcomes at any time in the newborn's medical history that is available when the data is extracted. When numbers allowed, certain disorders affecting the same organ system can be grouped to form a single, non-generic outcome (e.g. other CNS disorders). Table 2 provides a list of codes used to identify the presence/absence of each outcome in various embodiments.

Many embodiments extract data from clinical notes and calculate risk scores. To accomplish these tasks, gestational age at delivery and birthweight are extracted from clinical notes in the newborns' EHRs, in many embodiments. Free text in clinical notes can be systematically searched using regular expressions for “Gestational Age” and “Birth Weight”. The text associated with (e.g., following or preceding) these mentions can be extracted and converted into days for gestational age and grams for birthweight. When multiple clinical notes are available for the same newborn and values are discordant, the most commonly occurring value can be retained or the average across all the different values if two or more values appear with the same frequency.

Several neonatal risk scores have been developed to quantify the risk of mortality and/or severe outcomes in newborns. Most of these scoring systems have been derived from preterm newborns and target a single outcome, such as mortality. Many embodiments described herein predict a broader range of neonatal outcomes, including mortality, on all newborns, regardless of the gestational age at delivery.

In sequence-processing deep learning algorithms used in various embodiments, each element of the sequence (i.e., codes) can be represented as a real-value vector encoding the meaning of the element such that elements that are closer in the vector space are expected to be similar in meaning. These vectors, encoding the meaning of each potential element that can be found in the sequence, are called embeddings. Given the large number of unique codes, this approach can be preferable to one-hot encoding in which each code k would be represented by a K-dimensional vector of 0 s, except for the k-th element, which would be 1.

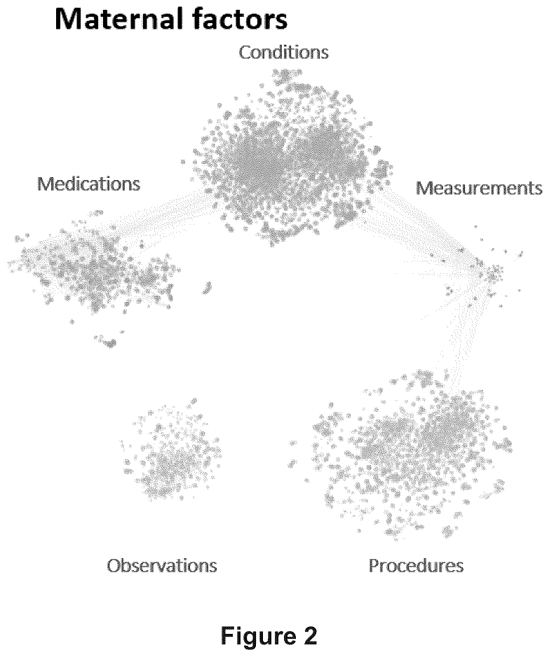

In various embodiments, inputs, such as the codes, are embedded to reduce the codes into a lower dimensional space. For example, codes may be reduced to 128-dimension space. As a non-limiting example, a sequence of n codes is converted into a 128×n matrix and fed into a bi-directional long short-term memory (LSTM) recurrent NN with 128 units. Recurrent NNs, are a class of NNs which use sequential data or time series data. Certain embodiments train global vector (GloVe) embeddings for all codes present in either the maternal or newborn's medical histories to reduce the codes into a 128-dimensional space. The GloVe model can be trained on the non-zero entries of a global code-code co-occurrence matrix, which tabulates how frequently codes co-occur with one another in a patient's EHR medical history. The main intuition underlying the GloVe model is that ratios of code-code co-occurrence probabilities have the potential for encoding some form of meaning. The obtained embeddings for codes can be projected into two dimensions, split by set (i.e. conditions, observations, medications and procedures) using tSNE for visualization purposes. Similarly, a two-dimensional tSNE map can be obtained for measurements. An exemplary tSNE map is illustrated in FIG. 2, where 20,172 codes present in the exemplary data is visualized. In FIG. 2, the size of the node is proportional to the metric described in the methods to assess feature importance, averaged across all outcomes; edges connect nodes whose correlation is among the top 1% of all correlations.

The encoded 128-dimensional space obtained can split into a training and a test dataset with a 60%-40% split. Subgroup discovery can applied in the training dataset containing the encoded 128-dimensional latent space and classification metrics were evaluated in the test dataset. Each dimension in the latent space can be discretized into groups (e.g., 2 groups, 3 groups, 4 groups, 5 groups, etc.) using appropriate quantiles to form the search space. The target concept can the AUPRC, so that subgroups identified were those where the AUPRC is the highest. AUPRC can be obtained from the ground truth presence/absence of a given neonatal outcome and the predicted score outputted by the AI model at delivery/birth. It should be noted that the foregoing embodiments are solely exemplary, and certain models and/or methods of training can include one or more layers; can have more or fewer units per layer; and/or the layers, data extraction, and/or training methodology can be optimized for computer performance or specific uses.

### Neonatal Outcome Prediction

Newborns delivered after 37 weeks have traditionally been considered a relatively low-risk group for adverse neonatal outcomes, with lower rates of neonatal morbidity and mortality compared to preterm newborns. Nevertheless, full-term newborns, especially those with cardiac, neurologic or genetic disorders are at increased risk for long NICU hospitalizations secondary to disease pathologies that overlap with preterm infants.

Certain embodiments can be used to predict neonatal outcomes. Such outcomes are be at different timepoints of gestation and/or post-delivery, such as any time from approximately 5 months before delivery to approximately 2 months after delivery. In such embodiments, a machine learning model (such as described herein) can be trained with:
(i) the sequence of codes from the maternal and newborn's medical history up to the timepoint of prediction,
(ii) maternal/newborn socio-demographic information, maternal measurements closest to the time of prediction and, when specified, gestational age and birthweight.

In some specific embodiments, input (i) included all the maternal EHR records up to the timepoint of prediction or delivery, whichever occurred first, plus newborn's EHR records up to the timepoint of prediction (only for models predicting outcomes after delivery/birth). Measurements in input (ii) were updated selecting the closest results to the timepoint of prediction or delivery (both within a 30-day time window), whichever occurred first, whereas gestational age and birthweight were added only in models obtained at delivery or onwards. For example, for the model trained using data available at delivery/birth, input (i) included all maternal EHR records up to delivery (i.e. the newborn date of birth) and no records from the newborn's medical history, input (ii) included measurements closest to delivery (within a 30-day time window), gestational age at delivery, birthweight plus maternal/newborn socio-demographic information. On the other hand, a model obtained one week after delivery was based on the maternal medical history up to delivery combined with the newborn's EHRs up to one week after birth [input (i)]; and on the maternal/newborn socio-demographic information, gestational age at delivery, birthweight and measurements closest to delivery formed input (ii).

In various embodiments, input (i), after code embeddings, is fed into a bi-directional long short-term memory (LSTM) recurrent neural network with 128 units, while input (ii) is processed by a dense one-layer neural network with 4 units. The outputs of these two networks are then concatenated and fed into a dense one-layer neural network with 64 units followed by a set of dense layers, one set for each outcome, consisting of two dense layers and a single-unit output.

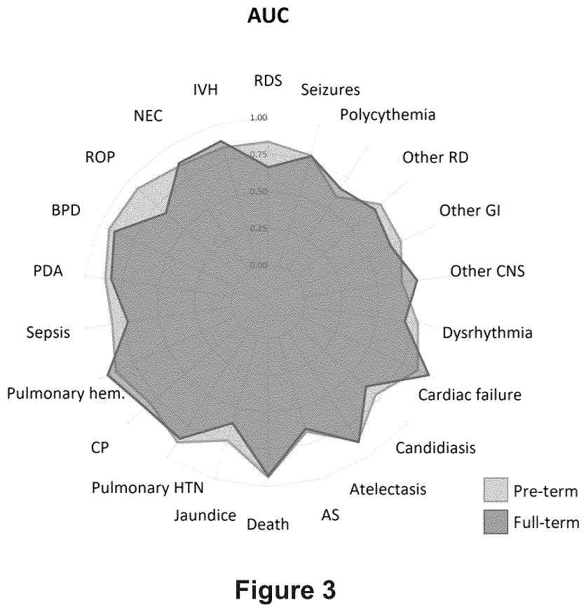

Many embodiments are capable of predicting outcomes in both preterm and full-term births. However, all 24 morbidity and mortality outcomes were more prevalent among preterm newborns, (n=3,639, 11.5%) than in full-term newborns (n=27,998, 88.5%) (See Tables 1 & 3). For example, in one embodiment, IVH prevalence was 5.1% and 0.2% in preterm and term newborns respectively, while NEC prevalence was 1.8% and 0.04%, respectively, and FIG. 3 illustrates the AUC for each outcome in preterm and term newborns, as the AUC is not dependent on the prevalence of the outcome. AUCs in term newborns were similar to those seen in preterm newborns for most of the outcomes; however, a decrease in the AUC was observed for RDS (0.661 in full-term vs. 0.833 in pre-term newborns), ROP (0.665 vs. 0.918), sepsis (0.707 vs. 0.814), hyperbilirubinemia (0.606 vs. 0.728), candidiasis (0.626 vs. 0.711), cardiac instability (0.683 vs. 0.773) and neonatal gastroesophageal reflux (0.659 vs. 0.734).

With such predictions, many embodiments can provide solutions to treat or ameliorate certain conditions, such as providing phototherapy (including intensity and/or time settings), respiratory strategies (e.g., type of gas, pressure, and/or other ventilator settings), and/or particular medications, supplements, medicines, or strategies that can be used prenatally (e.g., during gestation by the mother) or neonatally to prevent, ameliorate, and/or mitigate a neonatal morbidities or conditions.

### Neonatal Nutrition Supplementation

Certain embodiments can be used to generate recipes and/or formulations for intravenous (IV) nutritional supplementation bags for neonates. A significant problem is the ability to generate for nutritional supplement bags for premature babies. Many of such babies cannot absorb nutrients due to insufficiently formed digestive systems. Currently, supplementation takes the path of IV supplementation based on assessment of laboratory results. This process involves compounding custom bags for each individual neonate on an ad hoc basis and are susceptible to supply chain problems, contamination, human error, and/or any other issue involved in making and/or mixing components for IV supplementation.

To solve this problem, many embodiments are directed to nutrient bags for IV supplementation. Such nutrient bags can be developed from recipes and/or formulas designed using an autoencoder, such as described herein. Such models can be trained using neonatal EHRs, such as described herein, including formulations for nutritional supplementation contained within such EHRs.

Using a model trained as described, many embodiments provide recipes and/or formulations for standardized nutrient bags. Such embodiments can output any number of recipes and/or formulations, such as 1, 2, 3, 4, 5, 6, 7, 8, 9, 10, 15, 20, 25, etc. number of recipes/formulations. In various embodiments, there may be a reduced efficacy gained from additional recipes and/or formulations (e.g., 15 unique recipes/formulations may be 97% effective for supplementation, while 20 unique recipes/formulations may only provide 97.5% efficacy but would also require additional storage and/or manufacturing lines.

Nutrient bags, such as described herein, may be formulated as sterile IV bags. Such IV bags can be manufactured for later reconstitution with a diluent (e.g., sterile water, either locally sourced or sourced separately) or manufactured in liquid form, fully constituted for use. Additionally, some recipes may be catered to prevent and/or ameliorate specific conditions and/or disorders in a neonate, such as neurocognitive development, respiratory health, gastrointestinal health, eye health, and/or any other developmental condition or concern.

While the above describes methods to determine IV based nutritional supplementation, further embodiments can expand on this to provide oral health supplementation, such as through baby formulas (e.g., Similac®) or baby foods. Some embodiments provide recommendations and/or recipes for neonate nutrition, such as which specific foods (e.g., peas, carrots, beets, bananas, apples, etc.) and how much (e.g., 1 jar, ½ jar, etc.) to provide for a child to attain proper nutrition.

### Using Models

Embodiments are capable of using EHR obtained across multiple institutions (e.g., clinics, hospitals, children's hospitals, etc.) to predict neonatal outcomes. For example, many embodiments are able to obtain EHR for a child-producing individual, such as a female, a woman, a girl, a person with a uterus, and/or any other individual capable of giving birth. Such EHR can be obtained for the child-producing individual at any point before or during a pregnancy, such as for child planning, counseling, or other planning purposes on behalf of the child-producing individual. In some embodiments, such EHR is utilized by a medical practitioner, such as an obstetrician, gynecologist, neonatologist, and/or any other medical professional. In such embodiments, the outcome can be used to prepare medical treatments (e.g., surgery, antibiotics, etc.), nutritional planning, and/or any other act to benefit a child (pre- or full-term) that may be susceptible or prone to an adverse health condition.

EHRs can be obtained from public sources or proprietary data sources, such as a database. These data sources can be local (e.g., hard drive) or remote (e.g., a server accessed via network communication). Data sources can include hospital records, health system records, records from obstetricians, records from gynecologists, and/or electronically available records from any other medical or health source. Some EHRs can be compiled from a plurality of sources, such as when an individual receives care from multiple locations and/or medical professionals.

### EXEMPLARY EMBODIMENTS

Although the following embodiments provide details on certain embodiments of the inventions, it should be understood that these are only exemplary in nature and are not intended to limit the scope of the invention.

### Example 1: Longitudinal Risk Prediction for Maternal-Child Health Utilizing Artificial Intelligence and Electronic Health Records

### Methods:

### Data Sources

This is a cohort study anchored in routinely collected EHRs at Stanford Hospital and Clinics and the Lucile Packard Children's Hospital (California, US). The linkage of the EHRs from the two hospitals allows for a unique combination of serial maternal and neonatal data. All EHRs from inpatient and outpatient data were mapped to the Observational Medical Outcomes Partnership (OMOP) Common Data Model (CDM) version 5.3.1.16, 17 Data included patient demographics, provider orders, diagnostic, procedural, medication, laboratory test and clinical information collected during all inpatient and outpatient encounters. The study was approved by the Institutional Review Board of Stanford University (#39225).

### Delivery Cohort

First a cohort of 193,546 women was identified, where the women were aged between 14 and 45 years with at least one pregnancy-related record between 2014 and September 2020. A pregnancy-related record consisted of any record containing one of the codes identified in Matcho et al., (cited previously) to identify pregnancy episodes, broadly encompassing live birth, stillbirth, abortion (spontaneous and induced), delivery, pregnancy test, and ectopic pregnancy. Of the 193,546 women identified, 27,521 were linked to 32,356 newborns delivered at one of the two hospitals between April 2014 and October 2020. For the remaining pregnancies, no resulting product of delivery was found because this was either only a record of pregnancy testing with no actual pregnancy, delivered outside the two hospitals or the pregnancy was terminated. Of the 32,356 newborns identified, 2 were further excluded because they had less than 30 days of observation time available after birth or because there were no records for the respective mothers before delivery. The final dataset consisted of 32,354 newborns, with 3639 preterm, and 28,715 term infants from 27,519 mothers. Among the 32,354 pregnancies there were 644 twin pregnancies and 60 triplet pregnancy. Of the 27,519 mothers, 4,449 delivered multiple infants at various points in time; 4,087 delivered two newborns, 340 three newborns, 20 four newborns and 2 delivered five newborns in total.

### Maternal Medical History and Features Extraction

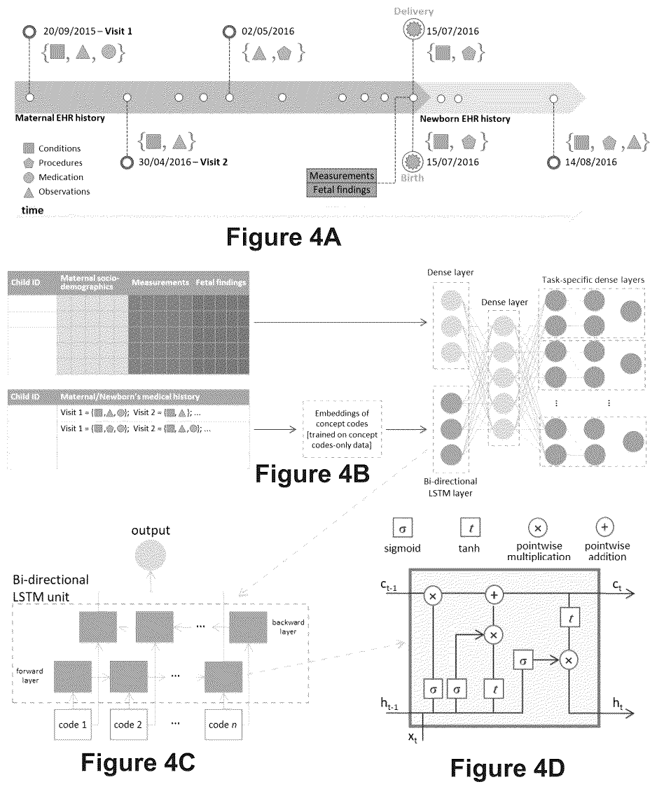

For each newborn, the entire maternal medical history available in the EHR up to delivery was extracted. This consisted of all conditions, observations, medications, procedures and measurements recorded under the mother's patient identification number. Different types of records were: 1) Conditions: presence of a disease or medical condition, 2) Observations: observed clinical sequelae obtained as part of the medical history, 3) Medications: utilization of any prescribed and over-the-counter medicines, vaccines, and large-molecule biologic therapies, 4) Procedures: records of activities or processes ordered by or carried out by a healthcare provider on the patient for a diagnostic or therapeutic purpose and 5) Measurements: structured values obtained through systematic and standardized examination or testing of a patient or patient's sample such as laboratory tests, vital signs, quantitative findings from pathology reports, etc., Conditions, observations, medications and procedures were organized by patient and time, and records corresponding to conditions used to identify newborn's outcomes were excluded to avoid potential leakage of information about the outcomes into the input data. The resulting entire sequence of time-ordered records, up to the timepoint of prediction (e.g. delivery, one week before delivery, two weeks before delivery, etc.), formed one of the newborn's personalized input to the model. In addition, the most common measurements, available in ≥10% of mothers, were extracted to form an additional newborn's personalized input together with maternal demographics (age at delivery and ethnicity), and, when specified, newborn's sex, gestational age at delivery and birthweight. The full list measurements utilized is reported in Table 1. For each measurement, the result closest to the timepoint of prediction (e.g. delivery), within 15 days before or after the point of prediction, was extracted (FIGS. 4A-4D).

### Newborn's Medical History and Outcomes

Similarly, the entire newborn's medical history of all conditions, observations, medications and procedures was extracted and organized by time. For each neonate, the resulting sequence of records was combined to the sequence of records of the respective mother (up to delivery/birth) to form the input data for models at points of prediction after delivery.

Moreover, as there was an interest in understanding whether artificial intelligence could accurately predict a wide range of neonatal outcomes and to fully interrogate the shared neonatal pathologies via the multi-task approach, a list of 24 neonatal outcomes was obtained as the presence or absence of any record related to each of these outcomes at any time in the newborn's medical history that was available when the data were extracted (i.e. January 2021 allowing a minimum of three moths of follow up). For the outcome death, we only considered deaths within two months after birth. These outcomes were selected among those subsumed by the ‘Neonatal disorder’ code (SNOMED code ‘22925008’) with enough cases (i.e. n≥100) to allow meaningful analysis and excluding transient disorders such as tachypnea, vomiting, electrolyte disturbance, etc. When numbers allowed, certain disorders affecting the same organ system were grouped to form a single, non-generic outcome (e.g. other CNS disorders). The list of codes used to identify the presence/absence of each outcome is reported in Table 2.

Data Extraction from Clinical Notes and Calculation of Neonatal Risk Scores

Gestational age at delivery and birthweight were extracted from clinical notes in the newborns' EHRs. Free text in clinical notes was systematically searched using regular expressions for “Gestational Age” and “Birth Weight”. The text following any of these mentions was extracted and converted into days for gestational age and grams for birthweight. When multiple clinical notes were available for the same newborn and values were discordant, the most commonly occurring value was retained or the average across all the different values if two or more values appeared with the same frequency.

Several neonatal risk scores have been developed to quantify the risk of mortality and/or severe outcomes in newborns. Most of these scoring systems have been derived from preterm newborns and target a single outcome, such as mortality. This approach more holistically aimed at predicting a broader range of neonatal outcomes, including mortality, on all newborns, regardless of the gestational age at delivery. The classification performance of the proposed model to that of two neonatal risk calculators was compared: the APGAR score and, the National Institute of Child Health and Human Development (NICHD)-Neonatal Research Network (NRN) mortality risk score. The APGAR score is routinely used in pediatrics and obstetrics to quickly evaluate the physical condition of all newborns after delivery. Clinical notes were systematically searched for regular expressions such as “APGAR scores:” or “APGAR totals” and the text following any of these regular expressions was extracted and further searched for mentions of “1 min:”, “1 minute:”, “one min:” or “one minute:” The APGAR score at one minute after delivery was then obtained by extracting the number following any of these regular expressions. Given that the APGAR score is a subjective measure of an infant's physical exam findings shortly after birth, while our proposed model is much more holistic, comparisons between models must recognize their significant differences and goals.

Information to calculate the NICHD-NRN mortality risk score was also obtained. In addition to gestational age at delivery, birth weight and newborn sex, multiple births and use of antenatal steroids were derived from the extracted conditions, observations, medications and procedures recorded in the maternal EHR history. Specifically, codes subsumed by the ‘Multiple Birth’ code (SNOMED code ‘45384004’) were used to identify multiple births, and codes related to ‘Betamethasone’ and ‘Dexamethasone’ (RxNorm codes ‘1514’ and ‘3264’) within two weeks before delivery were used to infer use of antenatal steroids. Among the calculators commonly used to assess survivability and risk for neurodevelopmental impairment in preterm newborns, the NICHD-NRN mortality risk score is frequently used in clinical practice. The score provides risk estimates for newborns delivered between 22 and 25 completed weeks of gestation, with a birth weight between 401 grams and 1,000 grams. The coefficient associated with the highest gestational age category (i.e. 25 weeks) was applied to preterm newborns born after 25 completed weeks (and before 37 weeks) in order to extend the calculation of the score to all preterm newborns in the study population. Similarly, coefficients for 22 weeks were applied when gestational age was less than 22 weeks. Once again, given that our model includes broad gestational age ranges and prediction of 24 queried neonatal outcomes, comparisons with the NICHD model must be interpreted with caution.

### Code Embeddings

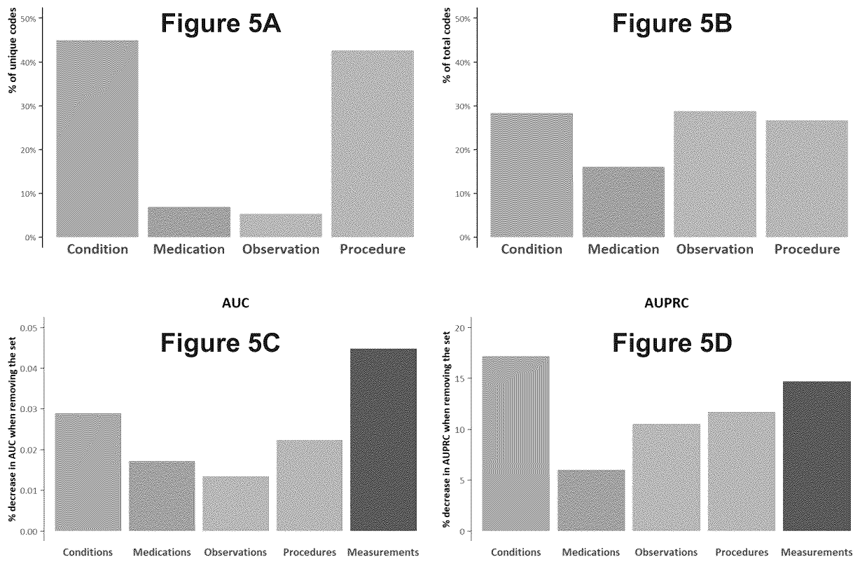

In total the maternal and newborn's medical histories contained 20,172 unique codes, of which 44.6% were condition codes and 43.0% were procedure codes. Out of the 11,182,582 records, 29.0% were records of conditions, 29.0% were procedures, 25.6% were observations and 16.4% were medication codes (FIGS. 5A-5D). In sequence-processing deep learning algorithms, each element of the sequence (i.e. codes) can be represented as a real-value vector encoding the meaning of the element such that elements that are closer in the vector space are expected to be similar in meaning. These vectors, encoding the meaning of each potential element that can be found in the sequence, are called embeddings. Given the large number of unique codes (K=20,172), this approach was preferred to one-hot encoding in which each code k would be represented by a K-dimensional vector of 0 s, except for the k-th element, which would be 1.

Global vector (GloVe) embeddings were trained for all the 20,172 codes present in either the maternal or newborn's medical histories to reduce the 20,172 codes into a 128-dimensional space.22 The GloVe model was trained on the non-zero entries of a global code-code co-occurrence matrix, which tabulated how frequently codes co-occur with one another in a patient's EHR medical history. The main intuition underlying the GloVe model is that ratios of code-code co-occurrence probabilities have the potential for encoding some form of meaning. The obtained embeddings for the 20, 172 codes were projected into two dimensions, split by set (i.e. conditions, observations, medications and procedures) using tSNE for visualization purposes. Similarly, a two-dimensional tSNE map was obtained for measurements (FIG. 2).

### The Multi-Input Multi-Task Deep Learning Model

Several multi-input multi-task deep neural networks were trained to simultaneously predict the 24 neonatal outcomes at different timepoints from 5 months before delivery up to 2 months after delivery. For each of these models, the inputs of the model are (i) the sequence of codes from the maternal and newborn's medical history up to the timepoint of prediction, (ii) maternal/newborn socio-demographic information, maternal measurements closest to the time of prediction and, when specified, gestational age and birthweight. Specifically, input (i) included all the maternal EHR records up to the timepoint of prediction or delivery, whichever occurred first, plus newborn's EHR records up to the timepoint of prediction (only for models predicting outcomes after delivery/birth). Measurements in input (ii) were updated selecting the closest results to the timepoint of prediction or delivery (both within a 30-day time window), whichever occurred first, whereas gestational age and birthweight were added only in models obtained at delivery or onwards. For example, for the model trained using data available at delivery/birth, input (i) included all maternal EHR records up to delivery (i.e. the newborn date of birth) and no records from the newborn's medical history, input (ii) included measurements closest to delivery (within a 30-day time window), gestational age at delivery, birthweight plus maternal/newborn socio-demographic information. On the other hand, the model obtained one week after delivery was based on the maternal medical history up to delivery combined with the newborn's EHRs up to one week after birth [input (i)]; and on the maternal/newborn socio-demographic information, gestational age at delivery, birthweight and measurements closest to delivery formed input (ii).

Input (i), after code embeddings, is fed into a bi-directional long short-term memory (LSTM) recurrent neural network with 128 units, while input (ii) is processed by a dense one-layer neural network with 4 units. The outputs of these two networks are then concatenated and fed into a dense one-layer neural network with 64 units followed by a set of dense layers, one set for each outcome, consisting of two dense layers and a single-unit output (further details in the Supplementary material).

Five-fold cross validation was performed in order to avoid overfitting to the data. First, newborns were randomly partitioned into five parts. Subsequently, the model was trained five times: each time the model was trained using inputs from newborns in four of the five parts as training/validation data while the remaining part was used as test data, so that predictions for each newborn come from a model trained without using data related to that newborn. Cross-validation area under the precision-recall curve (AUPRC) and under the receiver operating characteristics curve (AUC) were used to assess the classification performance of the model. The reference value for AUC, i.e. the AUC achieved by a random classifier, is always 0.5, regardless of the prevalence of the outcome; on the other hand, the reference value for AUPRC corresponds to the prevalence of the outcome and, therefore, differs from outcome to outcome. For visualization purposes, we also reported the fold increase/decrease of the AUPRC obtained by the AI model compared to the AUPRC of a random classifier; the prevalence of each outcome is reported in Table 4.

When measurements, gestational age and birthweight were missing they were imputed using the respective mean values in the available data. All the analyses were performed using R v3.6.3 and the multi-input multi-task deep neural networks were implemented using Keras through the R package ‘keras’. AI models were trained using a batch size of 512, Adam optimization, binary cross-entropy loss with early stopping (training was stopped after 10 consecutive epochs with no improvement in validation loss) or stop after 100 epochs.

### Robustness to Dataset Shift and External Validation

EHR data can be subject to changes in the patient population, clinical and administrative workflows, and updates in coding systems. These changes can lead to temporal dataset shifts that would impact the deployment of AI models and result into the degrading of the predictive performances over time. In order to test for any potential dataset shift, an experiment was in which the AI model at delivery/birth was trained using newborns born between 2014 and the end of 2018, and tested in those born in 2019 and 2020, separately. AUCs and AUPRCs were then compared from the original model to those obtained in newborns born in 2019 and 2020.

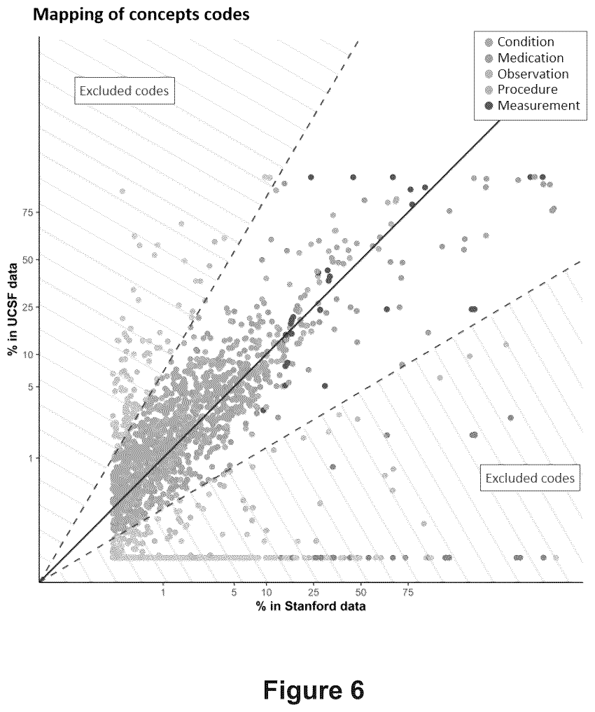

Additionally, a simplified model was trained for five selected outcomes (RDS, NEC, IVH, PDA and anemia of prematurity) and validated the performance in external EHRs from UCSF. Linked maternal-newborn EHRs including conditions, medications, procedures and measurements were available for 12,258 neonates in the UCSF EHR database. This model first identified 1,808 different OMOP CDM concept codes which were present in the maternal medical history up to delivery of at least 0.2% of pregnancies identified in the Stanford delivery cohort. These were mapped to the relevant coding system used for UCSF EHRs: ICD 9 and 10 for conditions, RxNorm for medications, CPT4 for procedures and LOINC for measurements. Mapping was done as indicated in the OMOP CDM concept relationship table. For each of the codes, the proportion of maternal medical histories in which these codes were present up to delivery were compared in Stanford and UCSF pregnancies (FIG. 6). To avoid bias due to the mapping of codes from different coding systems, subsequent analyses were restricted to the 850 concept codes for which the proportions in the two datasets was similar (i.e. when the proportion of maternal medical histories at UCSF in which the concept code was present was at least half and less than twice the same proportion at Stanford).

Binary variables indicating the presence/absence of these selected 850 concept codes in the maternal medical history up to delivery were generated in both Stanford and UCSF data. For each selected outcome, concept codes were ranked based on their association with the outcome (assessed using OR) in Stanford data and a logistic model with the top 10 codes plus gestational age was trained using Stanford data. These models were then tested in the USCF data and AUC and AUPRC were calculated. These simplified models served to verify the generalizability and transferability of the more complex multi-input multi-task model to external health care settings. External validation of the full model was not possible due to the difference in the coding system used.

### Subgroup Discovery

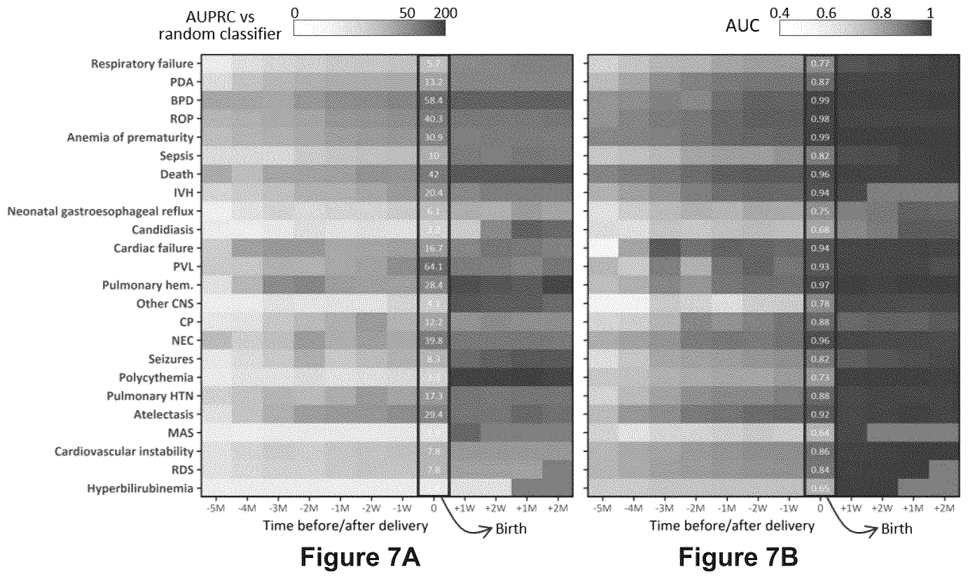

Subgroup discovery was used to identify subgroups of newborns for which the AI model at delivery/birth showed the highest predictive ability in terms of AUPRC. Subgroup discovery can be used for heterogenous study populations such as the one employed in this dataset. First, a 128-dimensional latent space of input (i) at delivery was obtained using a LSTM autoencoder (FIG. 7A). Autoencoders are a self-supervised learning model that can learn a compressed, lower dimensional representation of the input data. An autoencoder typically consists of an encoder and a decoder: the encoder model reads the input sequence; the hidden state or output of this model represents an internal learned representation of the entire input sequence that is then provided as an input to the decoder model that interprets it and reconstruct the input sequence. The input of the autoencoder consisted of the sequences of concept codes, i.e. input (i), after code embedding. These were fed into a 256-unit bi-directional LSTM layer, followed by a 128-unit bi-directional LSTM layer. The output of this layer is the encoded 128-dimensional latent space of the input data that was used to identify subgroups using subgroup discovery. A repeat vector layer was used as a bridge between the encoder and decoder modules, the decoder consisted of two bi-directional LSTM layers that mirrored the two layers of encoder. The bridge layer can represent a bottleneck layer in an autoencoder. In certain embodiments, certain details can be extracted from a bottleneck layer, such as for subgroup discovery.

Subgroup discovery is a data mining technique that identifies descriptions of data subsets showing an interesting distribution with respect to a pre-specified target. Given a dataset X and a search space S identified by a set of descriptors (i.e. variables), subgroup discovery finds and ranks subgroups of X where a target concept is high or low. The encoded 128-dimensional space obtained was split into a training and a test dataset with a 60%-40% split. Subgroups discovery was applied in the training dataset containing the encoded 128-dimensional latent space (FIG. 7B) and classification metrics were evaluated in the test dataset. Each dimension in the latent space was discretized into 2, 3, 4 and 5 groups using appropriate quantiles to form the search space. The target concept was the AUPRC, so that subgroups identified were those where the AUPRC is the highest. AUPRC was obtained from the ground truth presence/absence of a given neonatal outcome and the predicted score outputted by the AI model at delivery/birth. Beam search was used with a depth equal to 2, i.e. subgroups were identified by combinations of no more than two descriptors (e.g. dimensions of the latent space). AUPRC within each subgroup in the training data was calculated to rank subgroups26; subsequently ranked subgroups were progressively combined until they covered at least 30% of the tests dataset and classification metrics were calculated in the resulting set of subgroups.

### Associations Between Input Features and Outcomes

To investigate what information drives the predictions of neonatal outcomes, the importance of each EHR code was evaluated, also grouped in sets of conditions, medications, observations and procedures. Maternal medical history and measurements up to 1 week before delivery were considered to identify features contributing to the development of outcomes beyond those immediately preceding delivery and during labor.

First, a code set removal experiment was conducted. For each set of conditions, medications, observations and procedures, all codes belonging to that set (one set at the time) were removed from input (i) of the AI model derived 1 week before delivery. For each set, the AI model was then re-trained using the modified input (i) that excluded all codes from that set (but included codes from the other sets) and 5-fold cross-validated AUCs and AUPRCs were calculated. Moreover, to evaluate the importance of measurements, AUCs and AUPRCs were calculated for the AI model trained without input (ii), therefore including only input (i) with codes from all sets up to 1 week before delivery. For each set (conditions, medications, observations, procedures and measurements) the percentage decrease in AUPRC and AUC due to the removal of the set compared to the AUPRC and AUC of the AI model including all sets was calculated.

In addition, the importance of each EHR code was explored towards the prediction of each neonatal outcome. A total of 13,668 unique codes were found in maternal medical histories up to 1 week before delivery; of these 7,082 were present in less than five maternal medical histories and were therefore excluded from this analysis. For each of the 6,586 unique codes found in at least five maternal medical histories, a binary variable was created indicating the presence/absence of that code in the maternal medical history up to 1 week before delivery. Then, odds ratios were calculated for each of these 6,586 binary variables and for each of the 24 neonatal outcomes, alongside the respective p-values to assess their significance. Similarly, odds ratios were calculated for each of the measurements considered (using the results closest to 1 week before delivery) and each of the 24 outcomes. Each measurement was dichotomized splitting by the respective median and logistic regression was used to calculate odds ratios and the corresponding p-values.

To balance between the strength of the association, indicated by the odds ratio, and the statistical significance, and to distinguish between positive and negative associations, a new metric was obtained as follows. Odds ratios (or the inverse of their reciprocal, i.e. −1/odds ratio, for odds ratios <1) were multiplied by 1 minus the respective p-value. The obtained metric was capped to 10 (or −10) to reduce the impact of outliers. The obtained metric ranged from −10 (very strong negative association between the code and outcome, meaning that the presence of the code in the maternal medical history, or the measurement being above the median, reduces the risk of the outcome) to +10 (very strong positive association indicating that the presence of the code in the maternal medical history, or the measurement being above the median, increases the risk of the outcome).

### Assessing the Benefit of the Multi-Task Approach

The predictive performance of the multi-input multi-task model at delivery/birth (described above) was compared to that of 24 separate multi-input single-task models, each trained to predict one of the 24 outcomes of interest. Both the multi-task and single-task models had the same inputs with information available at delivery/birth, i.e. (i) the sequence of codes from the maternal medical history up to delivery, (ii) maternal/newborn socio-demographic information, maternal measurements at delivery, gestational age and birthweight. The single-task models had the same architecture as the multi-task model with a bi-directional LSTM layer for input (i) and a dense layer for input (ii), concatenated and then fed into a dense layer. While in the multi-task model this last layer was followed by one set of dense layers for each outcome, in the single-task models this was followed by only one set of dense layers, the set that is responsible for the prediction of that specific outcome. Five-fold cross validation ROC and precision-recall curves were derived, along with the respective areas under the curve (AUC and AUPRC), to compare the predictive performance of the single-task model compared to the multi-task model for each of the 24 outcomes.

Moreover, a separate experiment was conducted to show the benefit of the multi-task approach particularly with respect to correlated outcomes. A large discrepancy was noted between the performance of the single-task and multi-task models when predicting NEC. Therefore, two additional multi-task models were trained with the aim of monitoring changes in the ability to predict NEC. A multi-task model was trained to predict NEC and the outcome that was most strongly correlated with NEC, i.e. anemia of prematurity. Similarly, a multi-task model was trained to predict NEC and polycythemia, i.e. the outcome with the weakest correlation with NEC. Both these multi-task models had the same structure described for the main multi-task model with all the 24 outcomes, with two separate final sets of dense layers, one for NEC and the other for anemia of prematurity and polycythemia, respectively.

### Results:

### Maternal/Newborn Characteristics and Neonatal Outcomes

A total of 32,354 live births occurring from 2014 to 2020 from 27,519 unique women were included in the study. Maternal and newborn sociodemographic characteristics are reported in Table 4 along with the prevalence of each of the 24 neonatal outcomes, which ranged in frequency from 0.07% (PVL) to 46.4% (hyperbilirubinemia). Comparative outcome prevalence in term and preterm newborns is included in Table 3. Codes by category extracted from the EHR have been listed by percentage with conditions and procedures each composing over 40% of the overall feature set (FIGS. 5A-5D). The overall prevalence of the neonatal outcomes at our center was comparable to national averages.

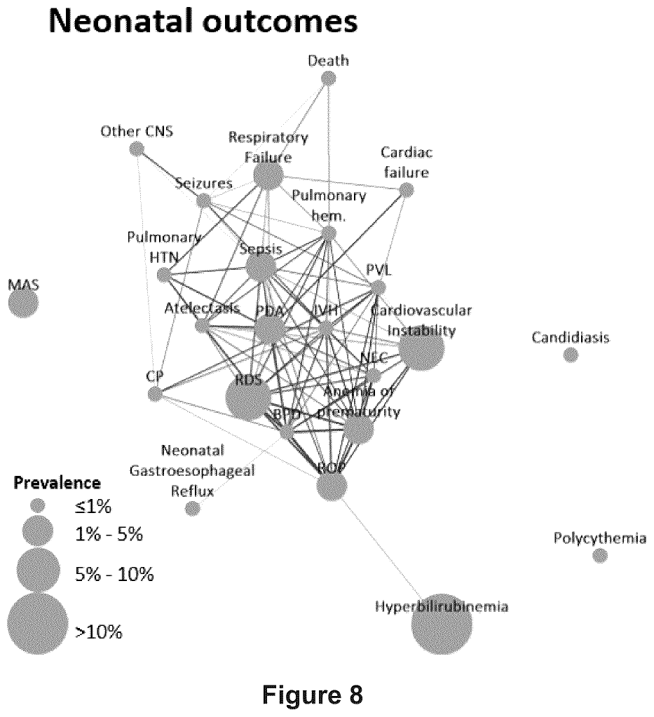

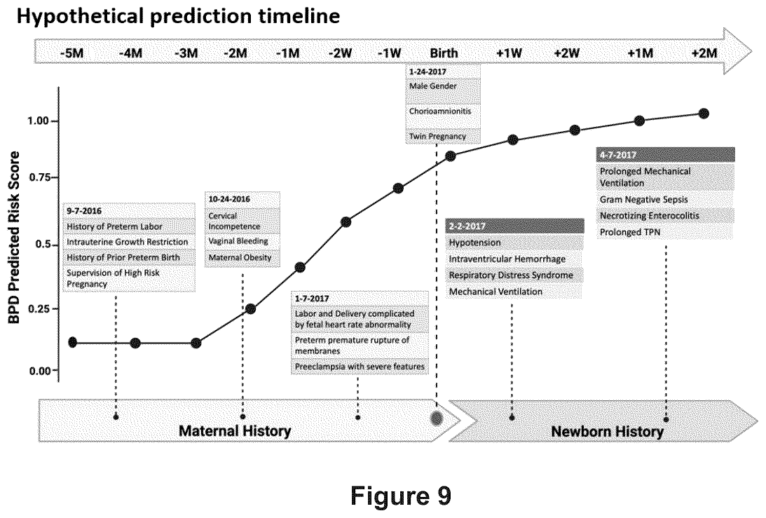

To investigate the relationship between the 24 neonatal outcomes, a correlation network was constructed showing tetrachoric correlations greater than 0.5 between pairs of outcomes (FIG. 8) and based on the maternal factors extracted from the EHR (FIG. 2). Several correlations were observed between various neonatal comorbidities with sepsis, pulmonary hemorrhage and atelectasis each showing correlations greater than 0.5 with 14 other outcomes. Conversely, the correlations of candidiasis, polycythemia and meconium aspiration syndrome (MAS) with any of the other outcomes did not exceed 0.5. FIG. 9 is a hypothetical prediction model for BPD incorporating known risk factors extracted from FIG. 2. In sum, the input data (FIG. 2) demonstrated strong internal correlations (FIG. 8) that justify the use of multitask learning and form the basis for hypothesis testing (FIG. 9) based on known clinical risk.

AI Model Predicts Neonatal Comorbidities Before, at and after Birth

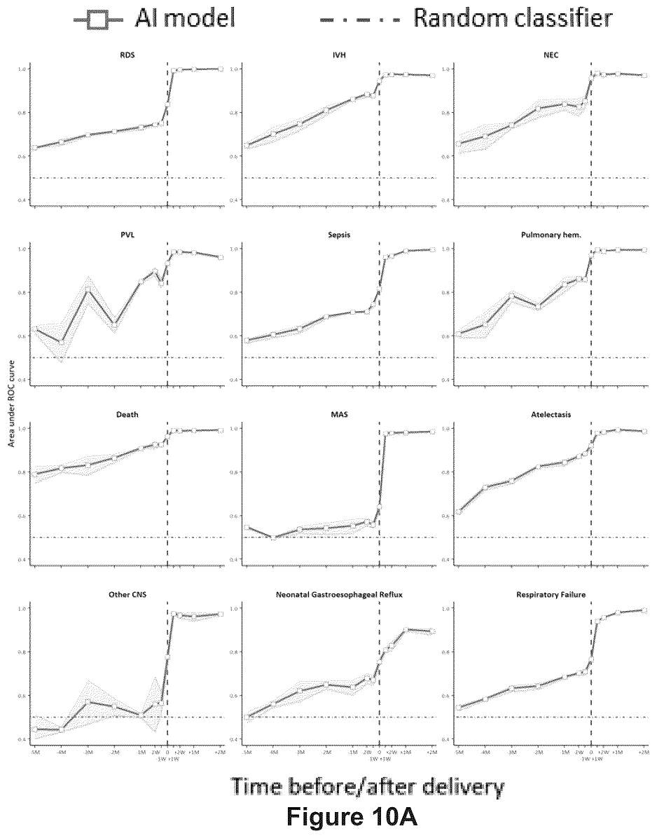

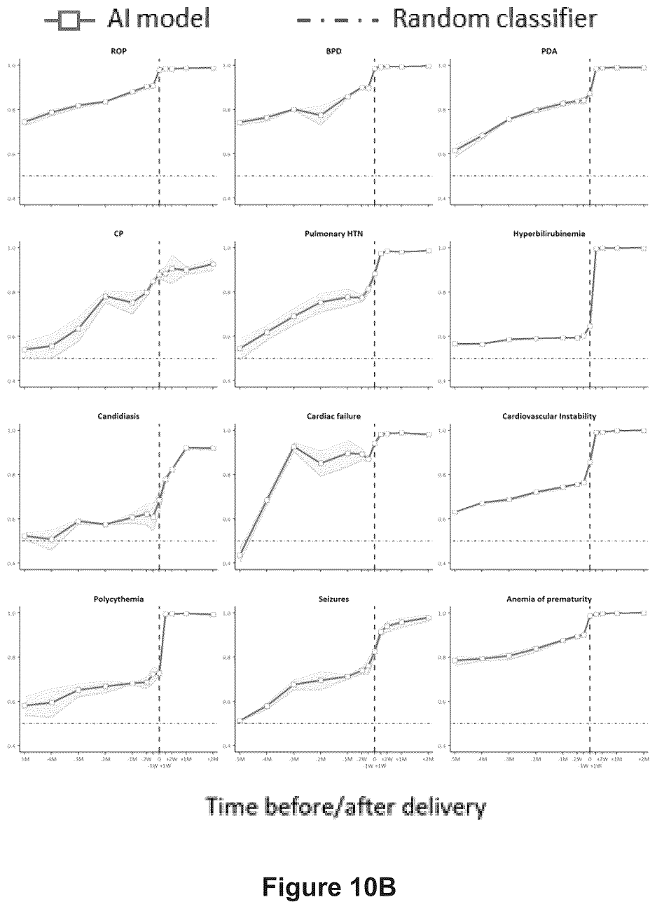

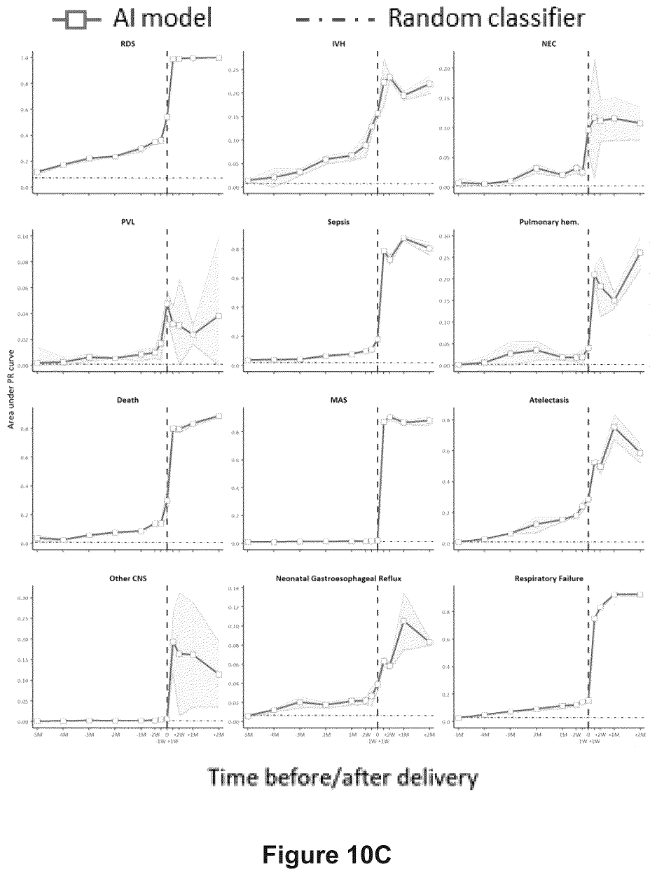

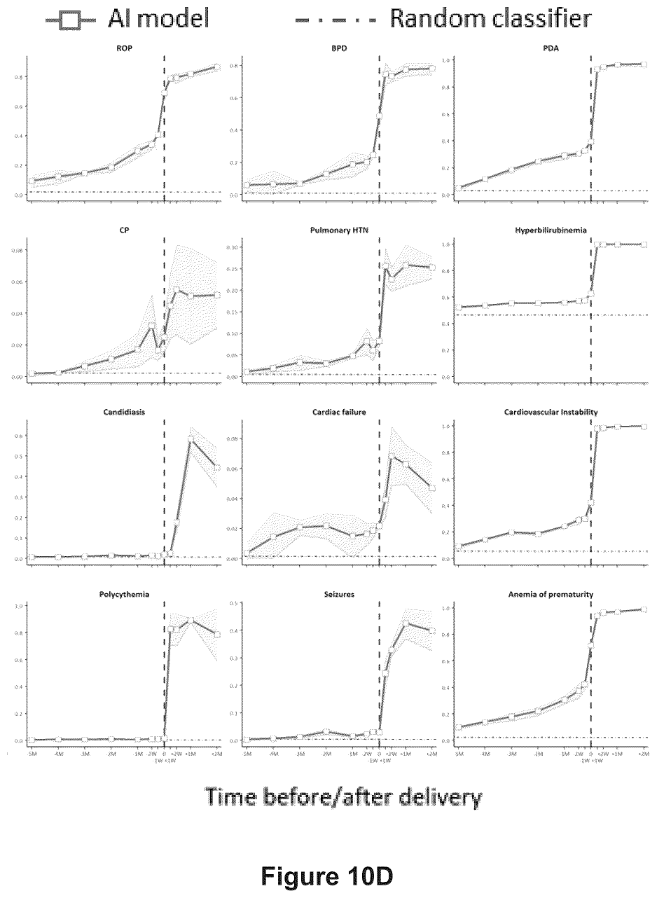

AUC and AUPRC (compared to a random classifier, equivalent to the prevalence of an outcome) of the AI model at different prediction periods from 5 months before delivery up to 2 months after delivery are reported in FIG. 7A. Predictions at delivery achieved AUCs ranging from 0.64 (MAS) to 0.99 (BPD and anemia of prematurity), with AUCs exceeding 0.9 for ten of the 24 neonatal outcomes considered (IVH, NEC, ROP, BPD, PVL, pulmonary hemorrhage, death, atelectasis, cardiac failure and anemia of prematurity) and between 0.8 and 0.9 for seven additional outcomes (RDS, PDA, sepsis, CP, pulmonary hypertension, cardiac instability and seizures). AUPRC was up to 62.7 times higher than that of a random classifier for PVL, 57.9 time higher for BPD, 41.4 times higher for death and 39.4 for NEC (absolute numbers are reported in FIGS. 10A-10D). The calculators developed include detailed outcomes data longitudinally such that a clinician can better quantify risk for the fetus or infant.

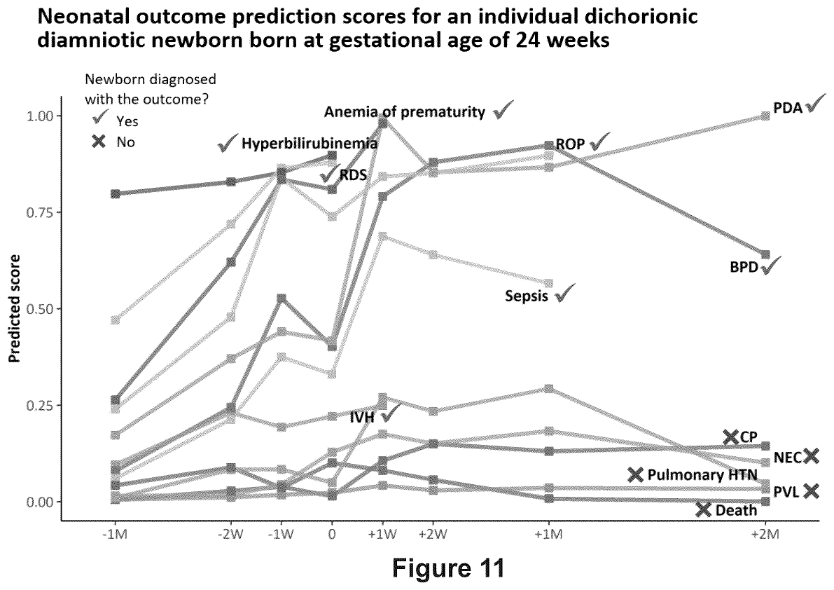

Importantly, the AI model showed good predictive performance before birth: one week before delivery the AUC was higher than 0.9 for death and ROP, and between 0.8 and 0.9 for IVH, NEC, BPD, PDA, PVL, pulmonary hemorrhage, CP, pulmonary HTN, atelectasis, cardiac failure and anemia of prematurity. Similarly, AUPRC at one week before delivery/birth was at least 10 times higher than that of a random classifier for twelve outcomes, in particular 30.6 times higher for BPD, 25.1 times for atelectasis and 24.8 and 24.4 times for ROP and PVL, respectively. FIG. 11 demonstrates the same AI prediction model for an individual patient born at 24 weeks and 2 days gestational age, incorporating this patient's unique maternal, neonatal and infantile time series data to formulate predictions on various outcomes related to prematurity. This patient was chosen to serve as an individual test on the model's ability to predict neonatal outcomes. This patient had EHR diagnoses of RDS, IVH (Grade I bilateral), BPD, Sepsis, PDA, Anemia of Prematurity, ROP and Hyperbilirubinemia. The individual prediction score at birth was highest for ROP, Anemia of Prematurity, RDS, Hyperbilirubinemia and Sepsis, all diagnoses for which the patient ultimately had. The prediction score at birth was lowest for IVH, NEC, Pulmonary Hypertension, CP, PVL, and Death. In sum, this data suggests that our AI model can predict individual outcomes on both a population and individual level.

### Predictive Ability is Consistent in Both Term and Preterm Newborns

Newborns delivered after 37 weeks have traditionally been considered a relatively low-risk group for adverse neonatal outcomes, with lower rates of neonatal morbidity and mortality compared to preterm newborns. Nevertheless, full-term newborns, especially those with cardiac, neurologic or genetic disorders are at increased risk for long NICU hospitalizations secondary to disease pathologies that overlap with preterm infants. Because of this, we sought to assess the ability of our AI model to predict neonatal outcomes in term newborns.

All 24 morbidity and mortality outcomes were more prevalent among preterm newborns, (n=3,639, 11.5%) than in full-term newborns (n=27,998, 88.5%-Table 4 and Table 3). For example, IVH prevalence was 5.1% and 0.2% in preterm and term newborns respectively, while NEC prevalence was 1.8% and 0.04%, respectively. FIG. 3 depicts the AUC for each outcome in preterm and term newborns, as the AUC is not dependent on the prevalence of the outcome. AUCs in term newborns were similar to those seen in preterm newborns for most of the outcomes; however, a decrease in the AUC was observed for RDS (0.661 in full-term vs. 0.833 in pre-term newborns), ROP (0.665 vs. 0.918), sepsis (0.707 vs. 0.814), hyperbilirubinemia (0.606 vs. 0.728), candidiasis (0.626 vs. 0.711), cardiac instability (0.683 vs. 0.773) and neonatal gastroesophageal reflux (0.659 vs. 0.734).

### AI Model is Robust to Temporal Dataset Shift and Hold Promise to Translate to Other Healthcare Settings

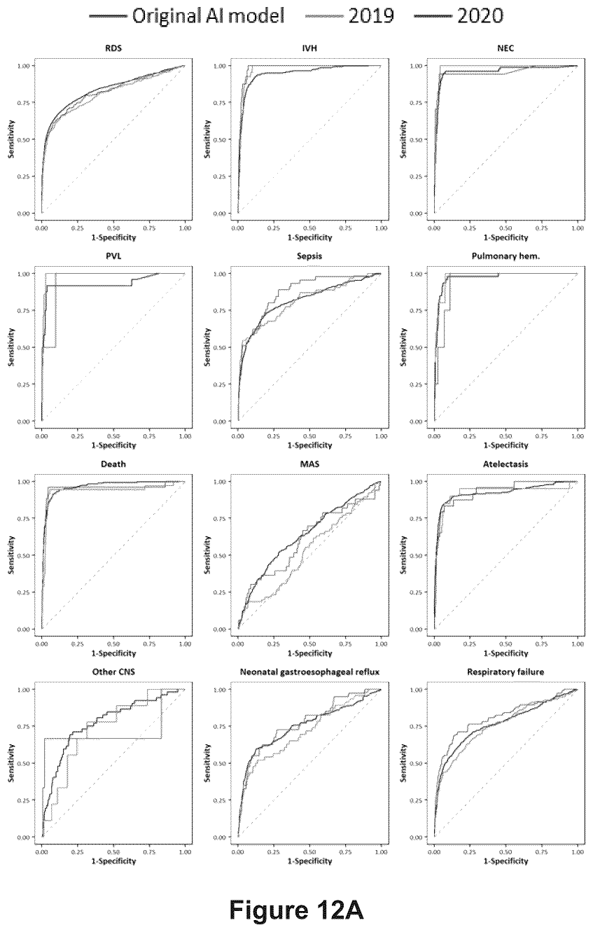

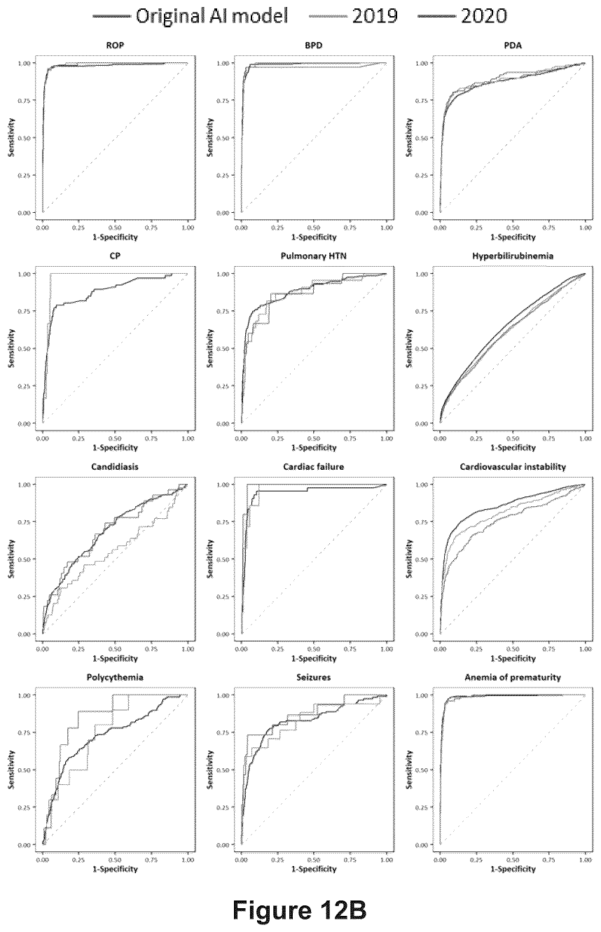

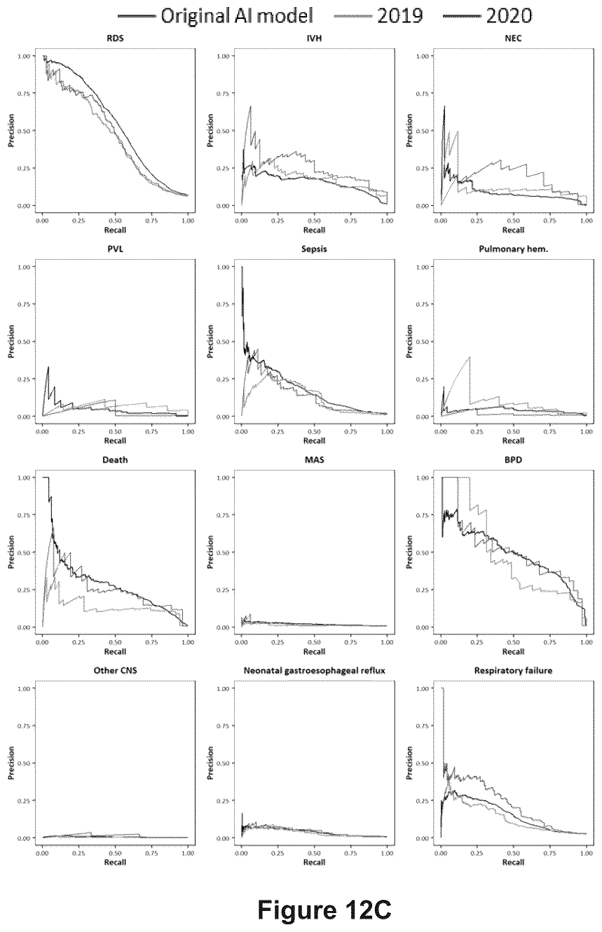

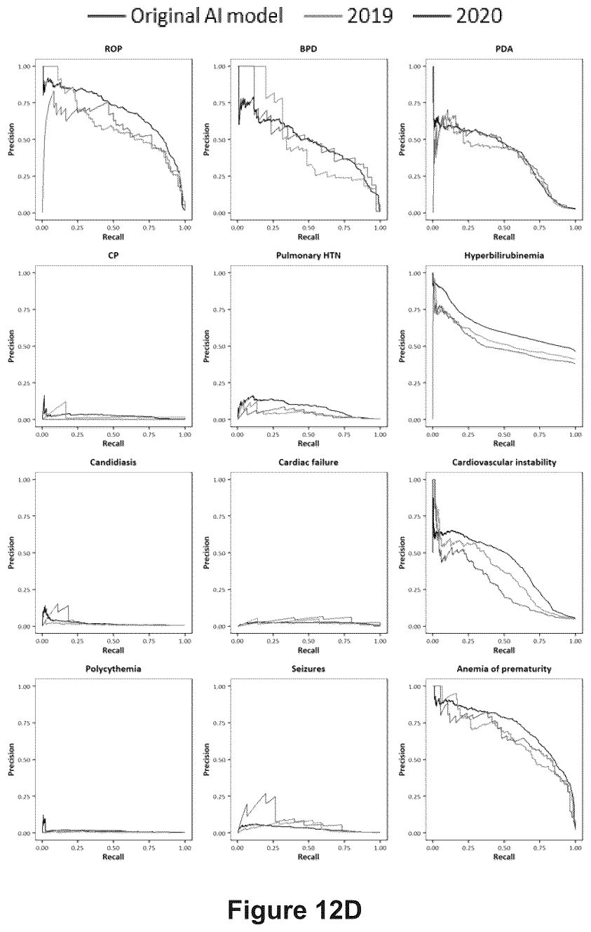

The AI model was robust to potential temporal dataset shifts; the performance of the AI model at delivery/birth trained on newborns born between 2014 and the end of 2018 (n=22,101), and tested in newborns born in 2019 (n=5,852) and 2020 (n=4,397) was similar to that of the original model (FIGS. 12A-12D). AUCs, AUPRCs and AUPRCs compared to a random classifier are reported in Table 5. Performances in 2019 and 2020 were in line with those seen for the original model; for example, AUPRC compared to a random classifier to predict NEC was 39.4 for the original model, and 44.0 and 45.1 in 2019 and 2020, respectively. For IVH, this went from 20.4 for the original model to 26.2 in 2019 and 35.6 in 2020.

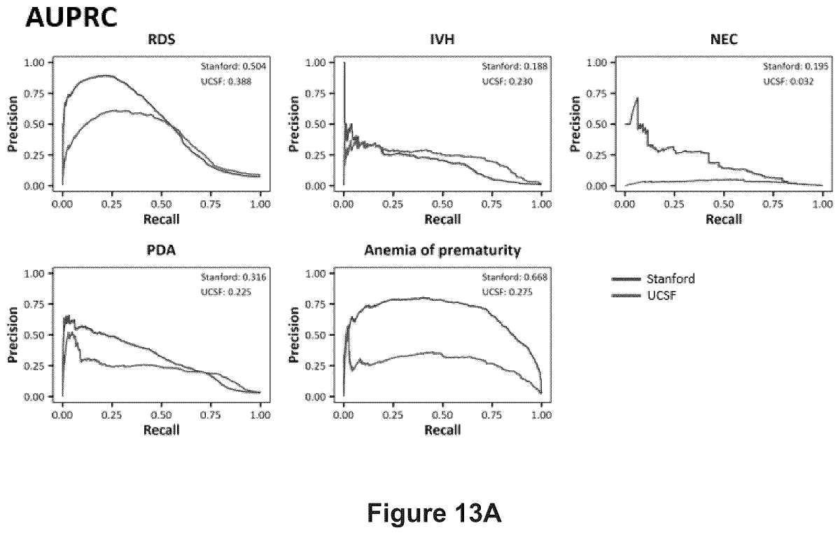

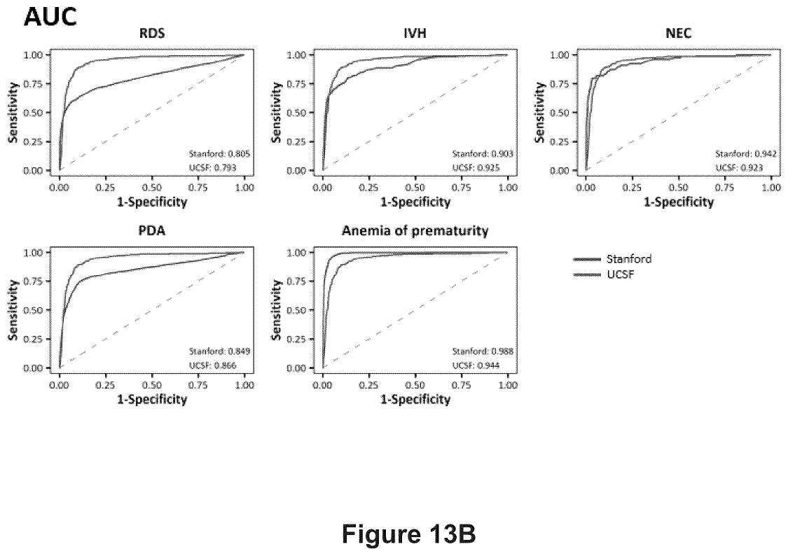

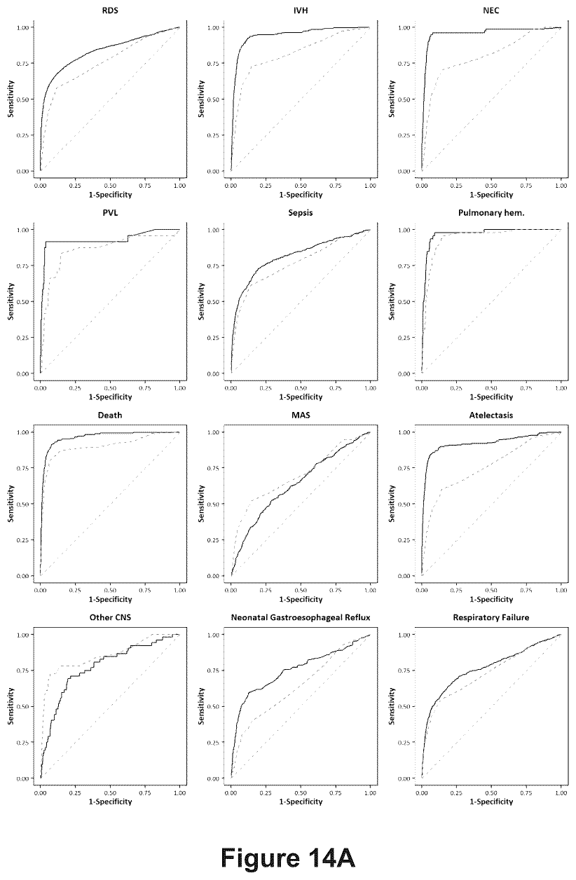

The simplified models built for validation in an external dataset were tested in 12,258 newborns obtained from UCSF EHRs and described in Table 6. Details of the simplified models trained using Stanford data are outlined in Table 7 and the results are visualized in FIGS. 13A-13B. AUCs of the models were similar across the two datasets for all the five outcomes (IVH: 0.903 in Stanford vs. 0.925 in UCSF; NEC: 0.942 vs. 0.923; anemia of prematurity: 0.988 vs. 0.944; RDS: 0.805 vs. 0.793; PDA: 0.849 vs. 0.866). AUPRCs were comparable for IVH (0.188 in Stanford vs. 0.230 in UCSF), PDA (0.316 vs. 0.225) and RDS (0.504 vs. 0.388); however, AUPRC dropped in the test data for NEC (0.195 vs. 0.032) and anemia of prematurity (0.668 vs. 0.275).

Subgroup Discovery Algorithm Identifies Subsets of Newborns for which the Predictive Ability of the AI Model is Improved

Using the 128-dimensional latent space of maternal EHR sequences, subgroup discovery yielded subsets of newborns comprising at least 30% of the whole study population where the AI model at delivery/birth achieved higher levels of precision and recall (FIG. 1 and Table 8). The subgroups identified achieved higher AUPRCs, in particular in comparison to a random classifier, for most neonatal outcomes. Above all, predictive ability of the AI model improved in subgroups identified for NEC (from 0.096 in the full dataset to 0.516 in the subgroup), ROP (from 0.690 to 0.860), BPD (from 0.487 to 0.641), PDA (from 0.394 to 0.466), hyperbilirubinemia (from 0.627 to 0.753) and anemia of prematurity (from 0.717 to 0.963). For most outcomes, subgroup discovery identified subsets of newborns with a lower prevalence of the outcome of interest compared to the full dataset. Since baseline AUPRC (i.e. the AUPRC of a random classifier) is equivalent to the prevalence of the outcome, it is important to compare the improvement compared to a random classifier. In subgroups identified, AUPRC of the AI model compared to a random classifier particularly improved for NEC (from 39.8 in the full dataset to 588.8 in the subgroup), anemia of prematurity (from 30.9 to 301.3), candidiasis (from 3.2 to 16.1), cardiac failure (from 16.7 to 64.3), atelectasis (from 29.4 to 103.2) and ROP (from 40.3 to 125.2). The subgroup discovery algorithm ultimately enhanced the predictive capability of the models, especially for outcomes that occur infrequently such as NEC.

### The AI Model Outperforms Current Used Risk Scores

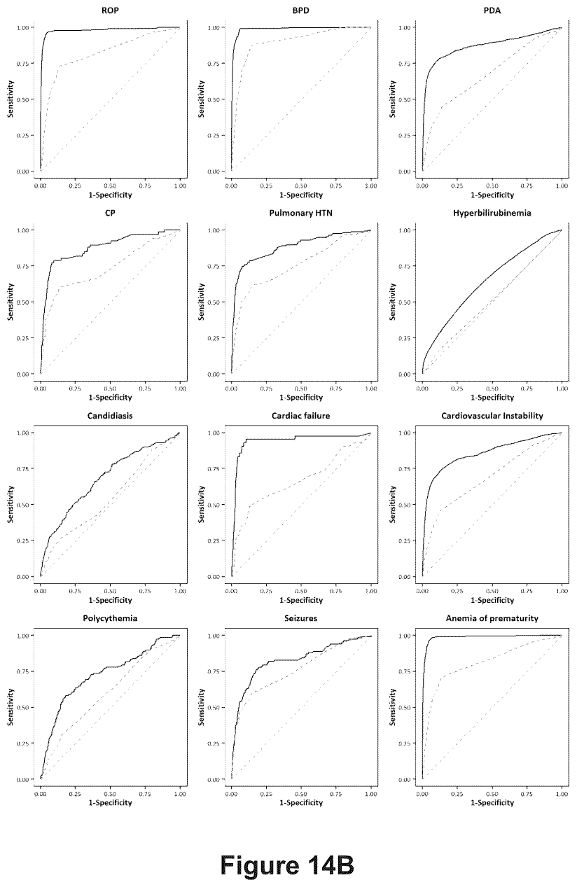

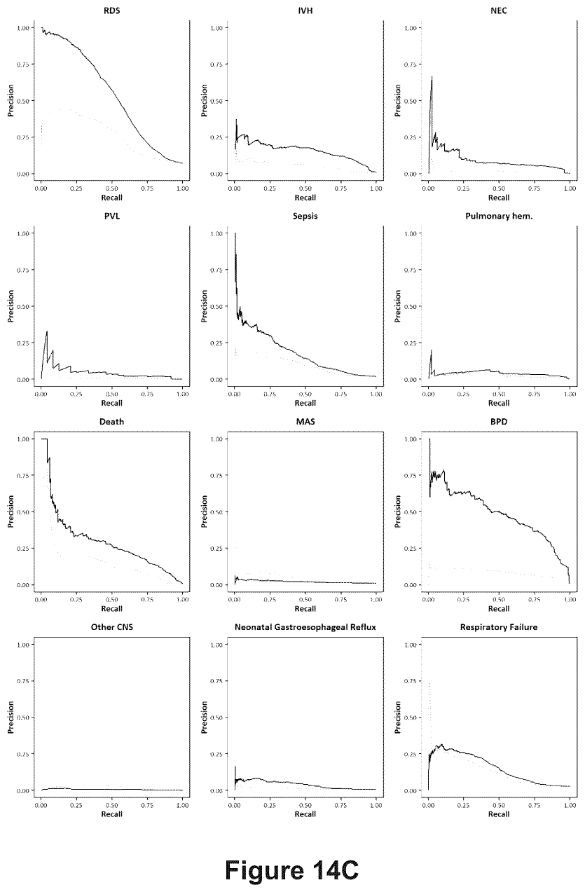

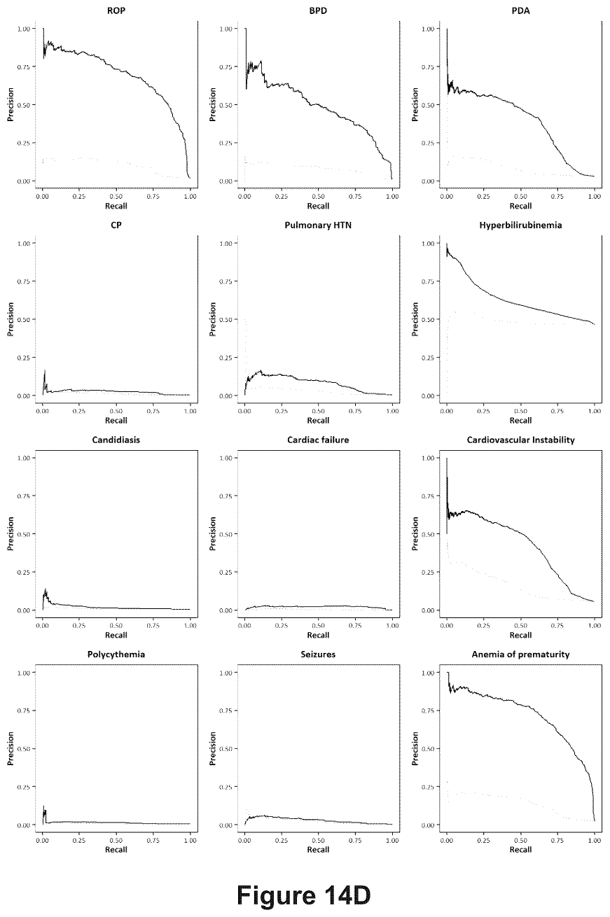

The AI model at delivery/birth largely outperformed the Apgar score at 1 minute both in terms of AUC and AUPRC as shown in Table 9 and FIGS. 14A-14B. AUPRC and AUC of the AI model was significantly higher than that of the APGAR score for 22 of the 24 outcomes, these include RDS, IVH, NEC, ROP, BPD, PDA, sepsis, pulmonary hemorrhage, CP, pulmonary HTN, hyperbilirubinemia and death (all p-values <0.001). When evaluated in preterm newborns, the AI model was notably better in terms of AUPRC and AUC also compared to the NICHD risk score (Table 10). The AI model showed a significant improvement compared to the NICHD score for all the outcomes with the exception of polycythemia and other CNS disorders. Of note, the AI model was designed to measure many additional outcomes beyond those measured by the NICHD-NRN or the APGAR score models. As such, comparisons must be interpreted with caution.

### Leveraging EHR Data to Explore Pathological Processes Underlying Neonatal Conditions

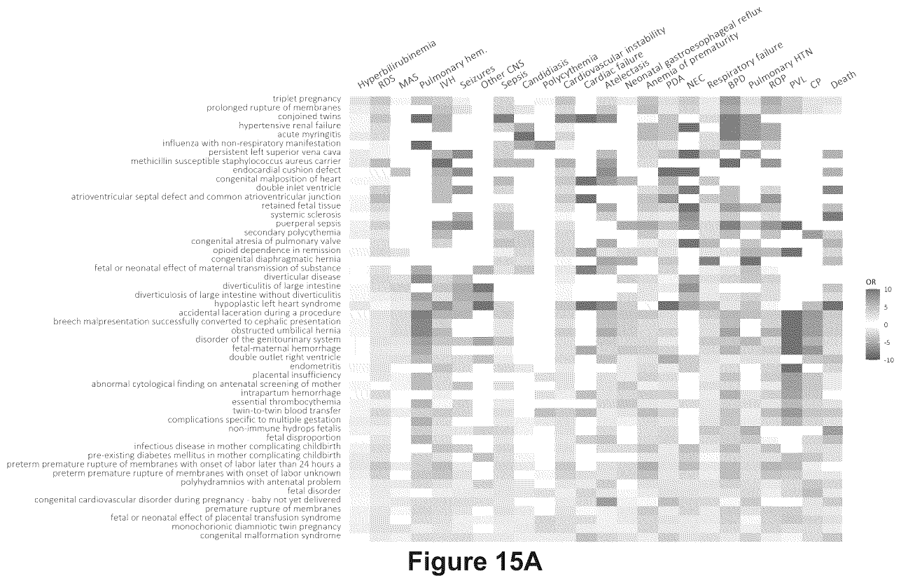

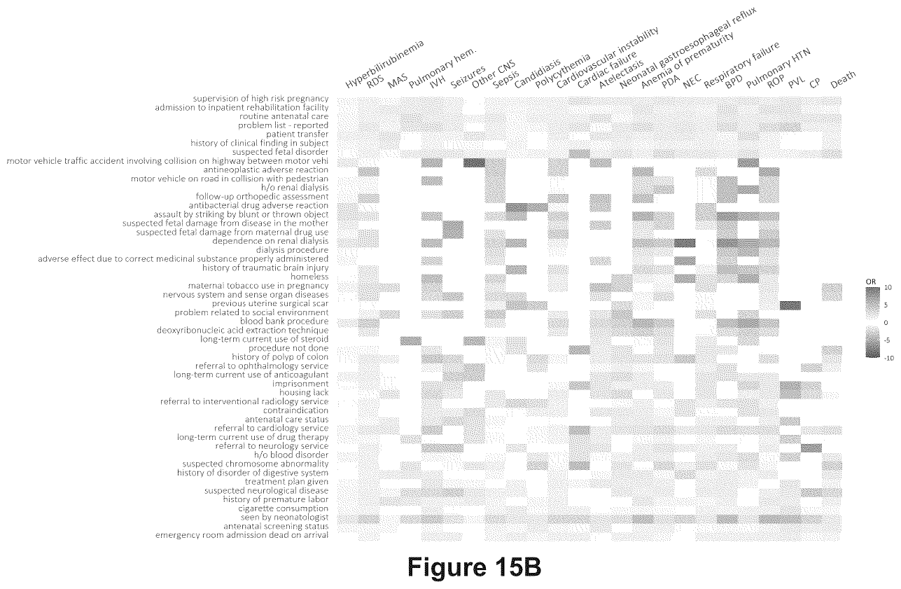

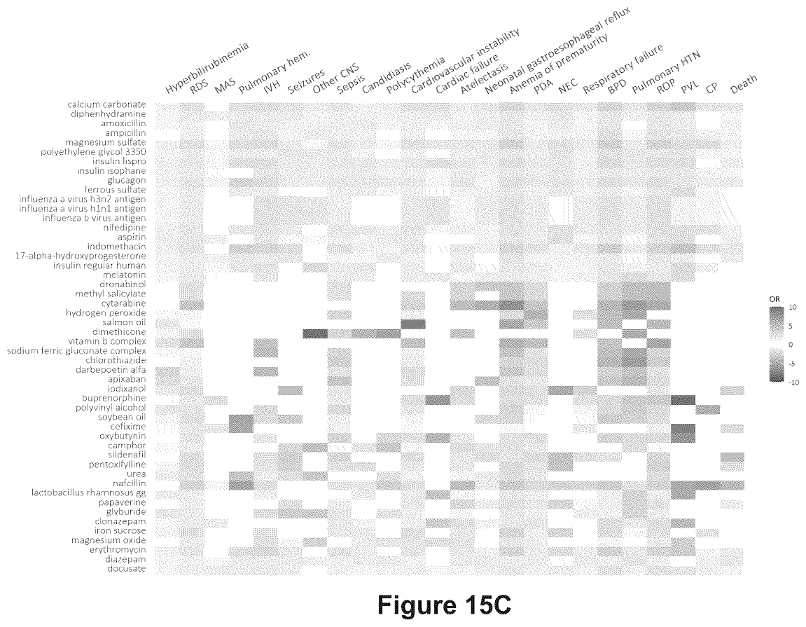

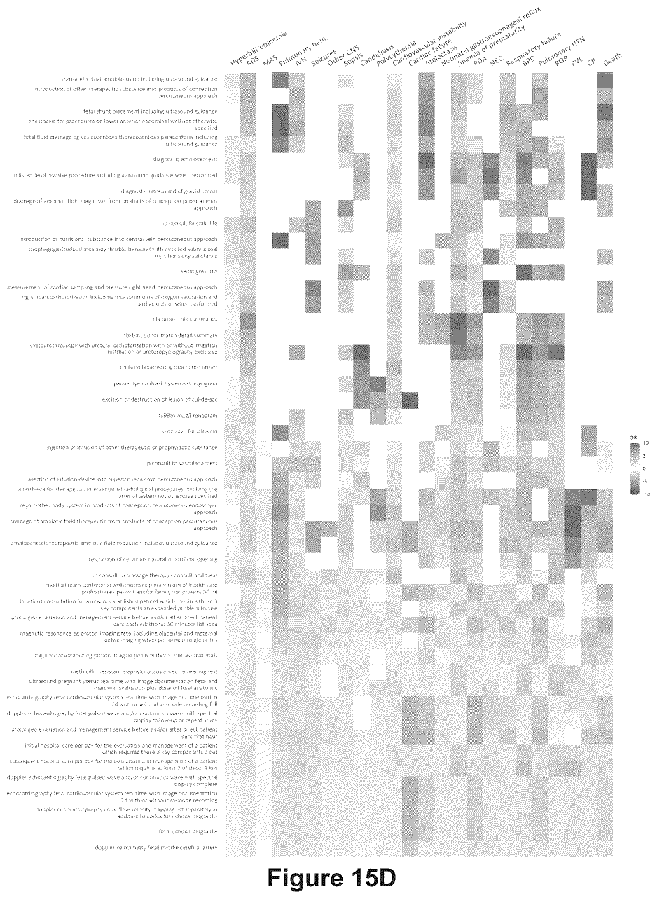

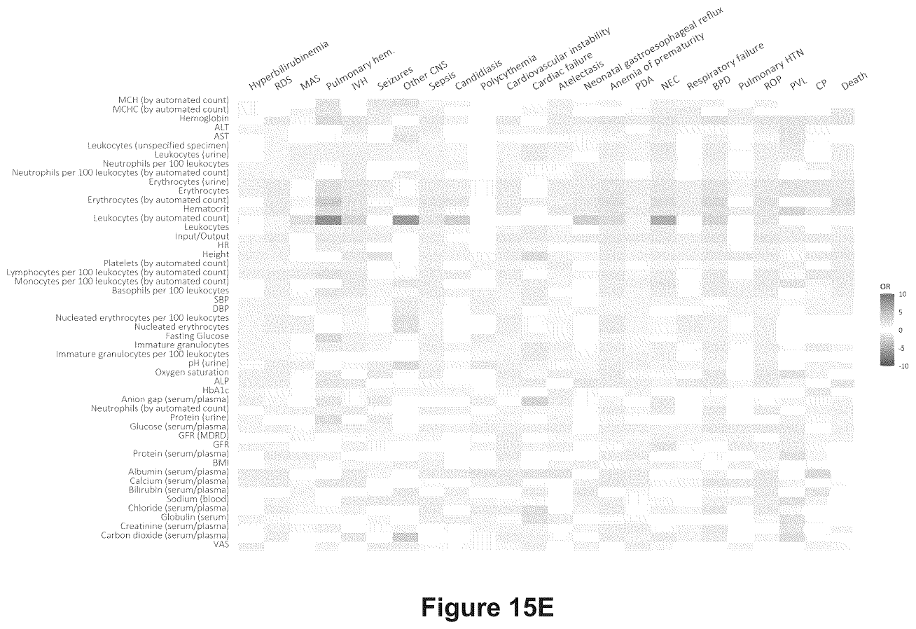

For each of the five identifiable categories of conditions, medications, observations, procedures and measurements, separate heatmaps are reported in the supplementary material (Supplementary FIGS. 15A-15D), showing odds ratios for the 50 codes (rows) for which the average odds ratio across all 24 outcomes (column) is highest and each of the 24 outcomes (columns). All associations between concept codes and neonatal outcomes can also be interactively queried, visualized and downloaded at the following link:

Of note, Supplementary FIG. 15D is a heat map of odds ratios between maternal laboratory measurements 1 week prior to delivery and the 24 neonatal outcomes. Higher laboratory values connote either a positive or negative odds ratio. Notable laboratory measurements that suggest a protective association against neonatal outcomes include serum albumin, serum protein, platelets, basophils, lymphocytes and eosinophils. This data suggests that there is interplay between the maternal immune system at 1 week prior to delivery and the relative health of the fetus that carries forward into the neonatal period and beyond.

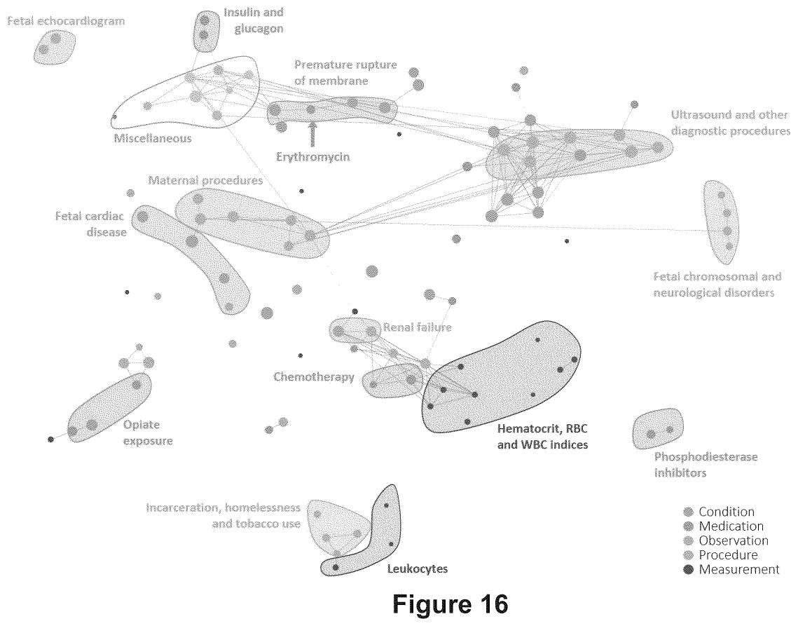

The correlation network in FIG. 16 shows the codes, and the interactions between codes, for which the average odds ratio across all 24 outcomes is highest. Among these codes strongly associated with neonatal outcomes were maternal outcomes including puerperal sepsis, PROM (prelabor rupture of membranes), preterm premature rupture of membranes (PPROM) with onset of labor unknown, PPROM with onset of labor later than 24 hours after rupture, opioid dependence in remission, fetal-maternal hemorrhage, various congenital heart diseases, renal failure and/or dependence on dialysis. In addition, there were codes that appeared to be novel risk factors for outcomes such as methicillin susceptible Staphylococcus aureus carrier status, renal failure, blood cell indices such as hematocrit, chemotherapy exposure and certain medications including phosphodiesterase inhibitors and opiates.

### Simultaneous Modeling of Neonatal Morbidities Improves Predictive Power

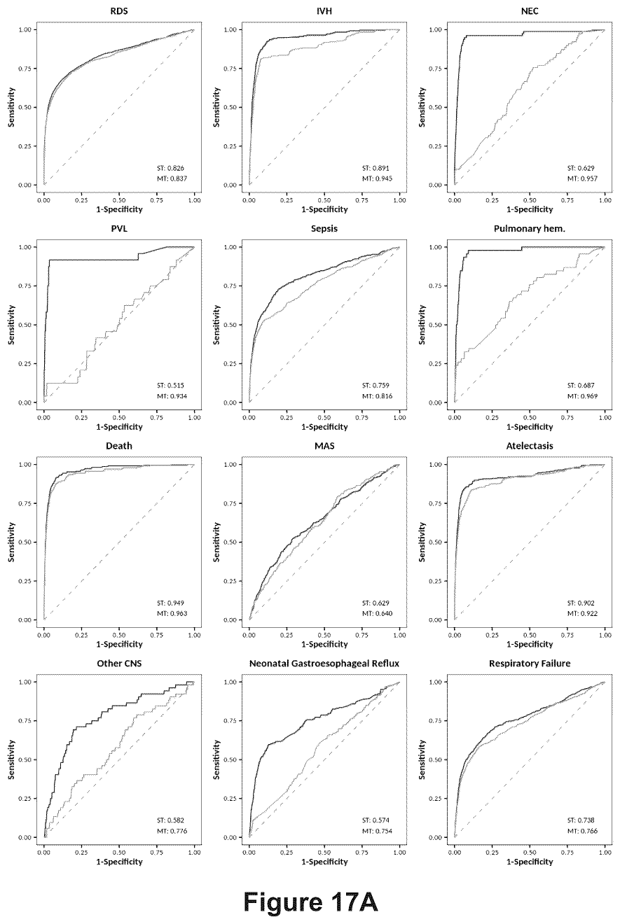

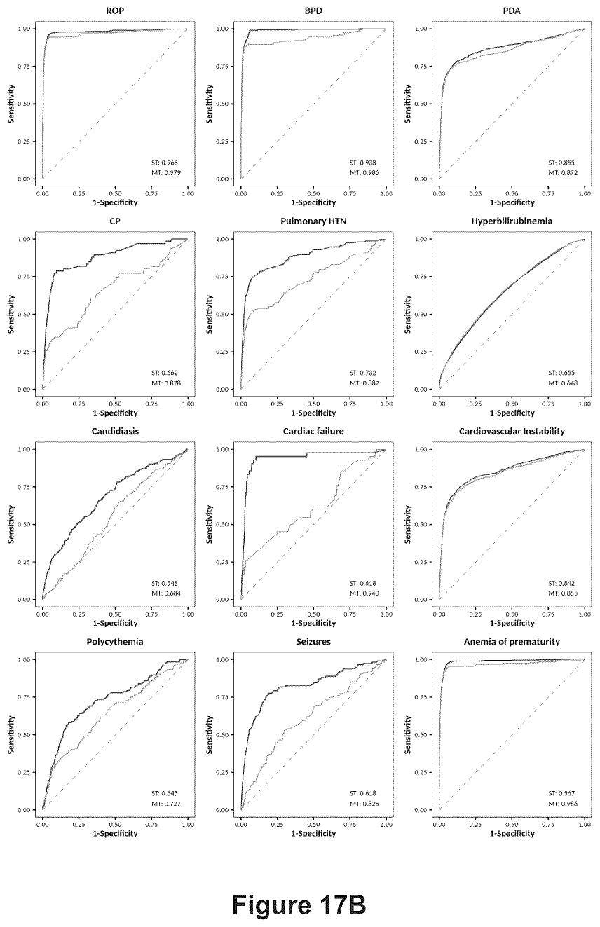

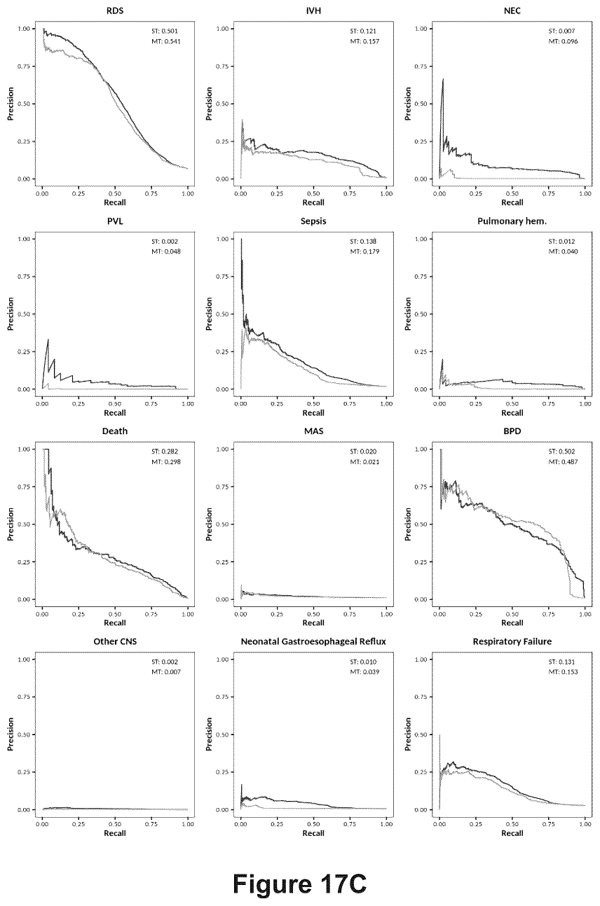

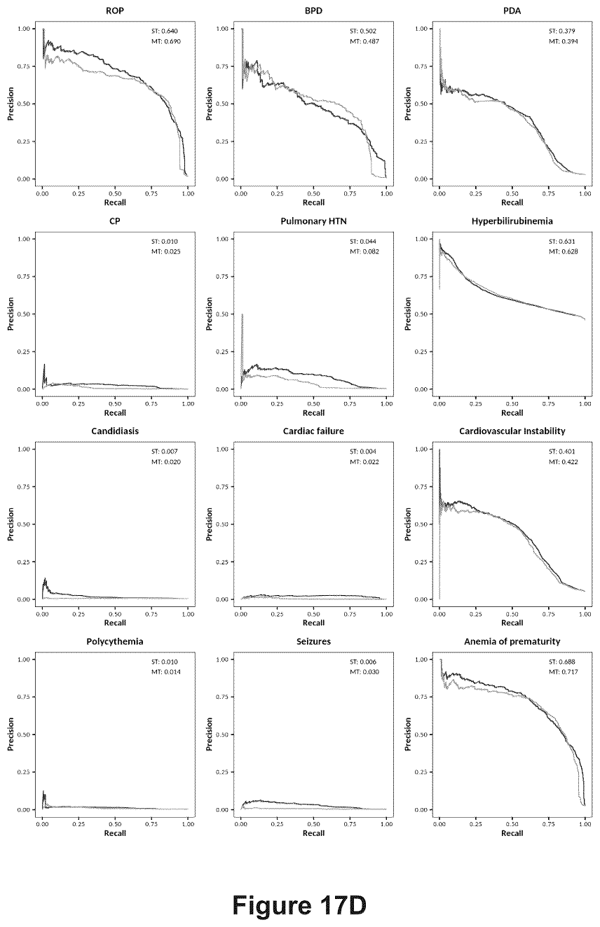

Results of the multi-task versus single-task experiment are reported in FIGS. 17A-17D, and show a clear improvement in both AUPRC and AUC of the multi-task model in comparison to the single-task model. The largest improvement, in terms of AUC, was observed for PVL (0.515 for the single-task model vs. 0.934 for the multi-task model), NEC (0.629 vs. 0.957), cardiac failure (0.618 vs. 0.940), and pulmonary hemorrhage (0.687 vs. 0.969).

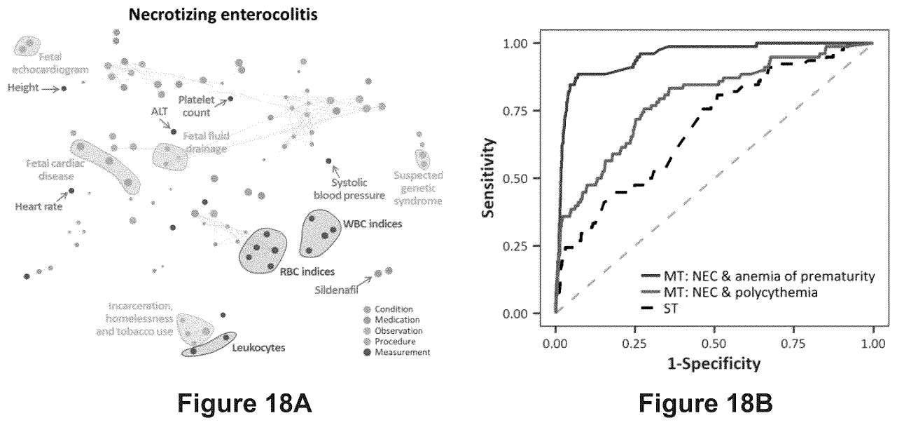

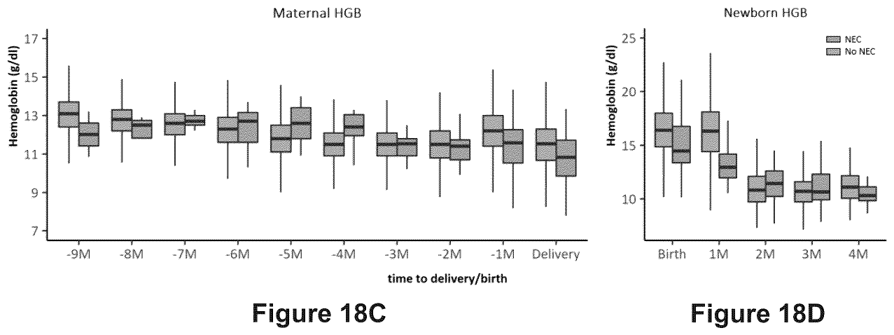

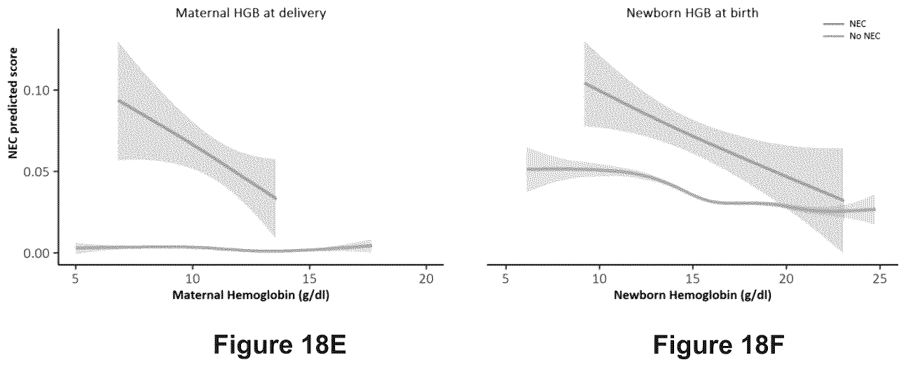

Given that there are a few neonatal outcomes that are exceptionally difficult for clinicians to predict, we undertook a detailed exploration NEC and related outcomes of interest. The relationship between maternal anemia, neonatal anemia, anemia of prematurity and NEC is depicted in FIGS. 18A-18F. AUPRC and AUC for NEC utilizing the single-task model were 0.007 and 0.629, respectively, as opposed to 0.095 and 0.957 obtained by the multi-task model. Tetrachoric correlation between NEC and polycythemia was 0.13, whereas that between NEC and anemia of prematurity was 0.75. The two-output multi-task model simultaneously predicting NEC and polycythemia achieved an AUPRC of 0.010 and an AUC of 0.636 for NEC, whereas the multi-task model predicting NEC and anemia of prematurity achieved an AUPRC of 0.056 and an AUC of 0.897 (FIG. 18A).

The AI Model Discriminates Newborns with IVH According to the IVH Grade

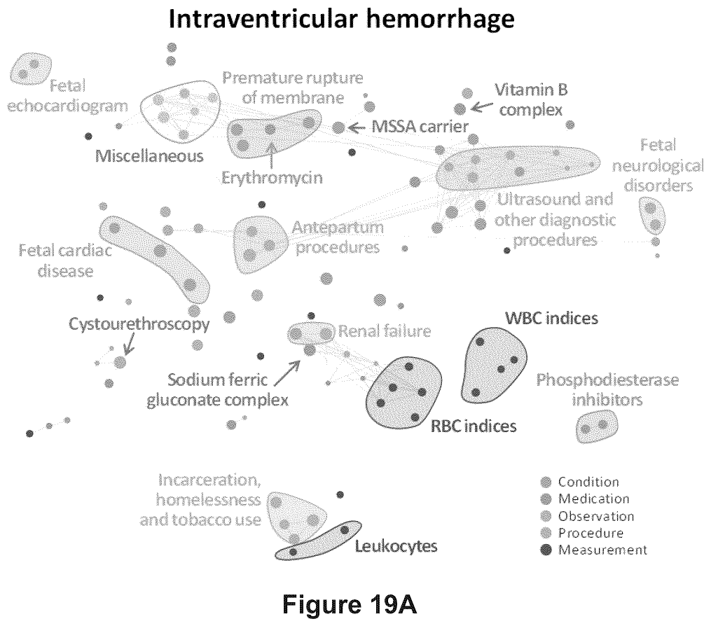

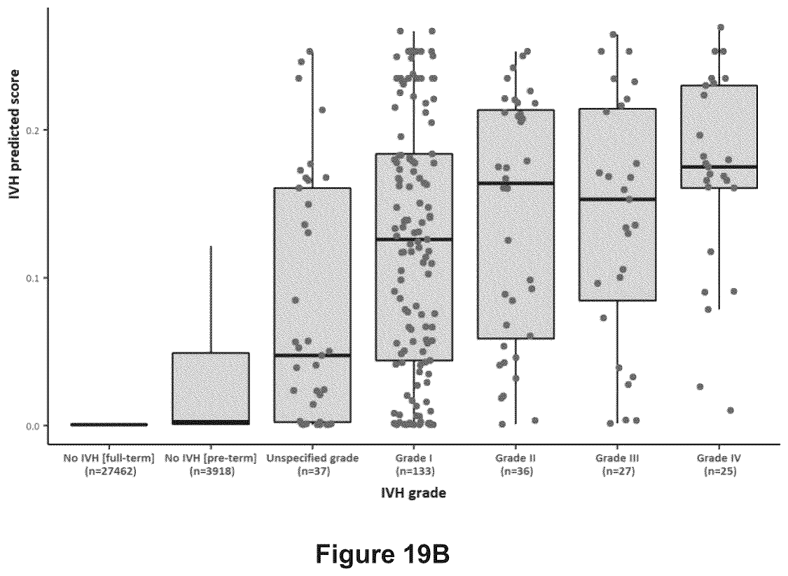

As IVH can also be difficult for clinicians to predict, we analyzed the IVH predicted score outputted by the AI model at delivery/birth for newborns with IVH. Newborns with IVH were grouped based on the IVH grade using records in newborns' EHR (the grade was unspecified when there were no records related to the grade of IVH). Importantly, the AI model was able to discriminate newborns based on the IVH grade; the IVH predicted score was, on average, lower for newborn with a lower actual IVH grade compared to ones with a higher IVH grade, with the average IVH predicted score increasing as the IVH grade increased (FIGS. 19A-19B). Given that IVH typically occurs within the first 96 hours of life, the IVH predicted score only included maternal and neonatal inputs that occurred at and prior to birth to avoid backwards contamination of the algorithm. In essence, the IVH predicted scores depicted in FIG. 19B suggest a dose-dependent relationship between the model inputs and the severity of IVH.

Discussion: Utilizing data at a single center collected from >27,000 mothers linked with >32,000 neonates between 2014-2020, it has been demonstrated that predictions of neonatal outcomes from various maternal conditions extracted exclusively from the EHR is possible. Prediction of neonatal morbidity remains a crucial problem across neonatal intensive care units. Almost all neonatal morbidities are classified based on clinical signs or resultant findings of disease rather than through the use of biomarkers representative of disease pathophysiology. Although prematurity is a significant risk factor for the emergence of a given neonatal morbidity, gestational age alone is not sensitive or specific for reliably predicting neonatal disease. Often, diagnosis occurs too late resulting in severe morbidity or mortality. Rapid and early diagnosis of neonatal disease is thus necessary to prevent severe morbidity and mortality. AI methodologies that can make predictions from large heterogeneous datasets have the potential to transform neonatal care by standardizing risk assessment across disparate patient populations. To our knowledge, this is the first investigation that has employed these approaches to interrogate the vast array of clinical data collected stored within both the maternal and newborn EHR to make clinically significant predictions of neonatal outcomes.

This work is distinct from prior risk prediction approaches utilizing the EHR in a few important ways. First, to our knowledge this is one of the largest sample sizes of maternal-infant records utilizing discrete clinical data including 5 categories of codes and extracted from the EHR. Second, using advanced machine learning methodologies, we have found novel associations between maternal conditions and neonatal outcomes that have clinical plausibility. Third, our findings demonstrate that fetal exposures to health conditions of the mother (anemia, certain medication exposures, social determinants of health) appear to increase neonatal susceptibility to diseases such as NEC, BPD, IVH, PDA, and CP. Fourth, we have built the first longitudinal clinical risk calculator (FIGS. 7A-7B and FIG. 11), incorporating real-time clinical data to predict neonatal outcomes beginning before birth and extending chronologically until 2 months of age. This calculator has the potential to transform clinical care in a number of different ways including: 1) minimizing inter-individual variability in management among providers; 2) providing individualized care based on a standardized risk assessment tool; 3) understanding longitudinal population level risk applied to individuals; 4) assist in targeting individual patients most appropriate for enrollment into translational and clinical trials based on longitudinal risk for a given disease and 5) allow clinicians to make better informed real-time assessments of their patients and pursue interventions or therapies in a timely fashion.

Most prior clinical risk prediction models employ algorithms based on a set of known risk factors, captured at a singular point in time utilizing data from large cohort studies. In adults, examples include the Atherosclerotic Cardiovascular Disease calculator, the CHA2DS2-VASc score for thromboembolic risk in atrial fibrillation, and Model for End-Stage Liver Disease for prediction of survival in patients with various forms of liver failure (insert citation). Within neonatology, calculators frequently used include the NICHD-NRN calculator, the BPD outcome estimator, the outcome trajectory estimator, the clinical risk index for babies (CRIB) and the Score for Neonatal Acute Physiology (SNAP) (insert citation). All of these predict survivability and other morbidities related to preterm birth and/or critical illness. But these calculators rely on information collected shortly before or after birth, making them difficult to rely on longitudinally. This is problematic given the lengthy hospitalizations of many critically ill neonates and the variable latencies for the most prevalent diseases. For instance, preterm birth often requires a 3-6 month NICU hospitalization.27 Thus, the need for risk prediction calculators that incorporate longitudinal data is crucial, as risk in this population is dynamic with an ever-increasing set of additive variables that serially accumulate and interact.28 Among the best known examples of a time-based clinical risk-prediction tool that is utilized for pediatric patients is the hour-specific nomogram for hyperbilirubinemia risk assessment tool.29 This hour-specific nomogram and calculator allows clinicians to determine if the level of serum bilirubin meets criteria for phototherapy and/or double-volume exchange therapy. This support tool is highly applicable, widely used and broadly lauded for its ease of use, all desirable characteristics that have resulted in widespread adoption. But this tool is solely used for management of hyperbilirubinemia. Our EHR-based longitudinal clinical risk prediction tool (FIGS. 7A-7B and FIG. 11) has combined many of the advantageous elements of other calculators, including the ability to project risk for multiple morbidities concurrently while incorporating maternal, and neonatal data simultaneously.

The longitudinal nature of our risk-assessment tool, combined with the comprehensive nature of the clinical data extracted has enabled us to uncover novel maternal factors and conditions associated with neonatal risk (FIGS. 18A-18F and FIG. 19A). In fact, we have found that risk for NEC in infants is highly associated with conditions that stem from chronic medical illness in mothers, including maternal anemia, social determinants of health (homelessness, incarceration), and certain prenatal maternal medications including indomethacin and sildenafil (FIGS. 18A-18F). Risk for IVH is associated with maternal factors including opiate exposure, renal failure and methicillin sensitive Staphylococcus aureus carrier status (FIG. 19A). Further studies are needed to validate these findings. Nevertheless, we believe these to be important findings insofar as risk related to prematurity is classically believed to originate from factors such as gestational age, birthweight and sex-static categories related to anthropometrics, rather than dynamic maternal conditions that likely impact underlying biology reflected at the maternal-fetal interface and subsequently impacting the fetus.

In addition, these findings lend nuance to the notion of clustering of acquired diseases of prematurity. Infants born prematurely often experience a co-occurrence of morbidities including BPD, NEC, ROP, cerebral palsy (CP) and sepsis. FIG. 9 demonstrates overlapping patterns of disease as evidenced by the large number of interconnected lines between each of the 24 different neonatal outcomes. RDS, anemia, BPD, sepsis, and NEC all highly correlate with one another as outcomes of prematurity. These outcomes can be predicted in aggregate based on the clinical trajectory of a maternal pregnancy but can also be identified individually, ie., for IVH (FIGS. 19A-19B). Indeed, our model demonstrates that IVH grade, based on the Papile grading system, can be predicted at birth with increasing accuracy with increasing severity of IVH. This suggests that the multi-task approach is capable of categorizing outcomes in a manner similar to what has been corroborated by clinical epidemiologic research.30

NEC is a relatively rare disease even in neonates born prior to 28 weeks' gestation (incidence 4-10%) thereby making it difficult to characterize and prospectively study. The current study is one of the largest investigations of NEC risk, that combines maternal and neonatal factors in a unified prediction. We found that in the multi-task approach, anemia and/or anemia of prematurity was highly correlated with NEC (FIGS. 9, 18D, and 18F). This observation adds to prior evidence of association from smaller studies. Severe anemia (i.e. hemoglobin <8) has been postulated as one of the first events in a cascading series of bowel-hypoxia-ischemia. These findings suggest that the hemoglobin level in either mothers or neonates may also be associated with the development of NEC, as lower hemoglobin levels in mothers shortly after conception (−9M) was correlated with neonates who later developed NEC (FIG. 18C). Additionally, neonatal hemoglobin level at birth, along with greater variance in the hemoglobin levels over the first two months of life was also associated with development of NEC (FIG. 18F). Impaired placental-fetal transfusion as may occur with partial cord occlusion or early umbilical cord clamping, may be the first sentinel steps in a sequence of events that contribute to anemia, transient hypovolemic shock and ischemic stress that later predisposes for later NEC. Various studies have demonstrated associations between anemia severity and NEC, some even suggesting a dose-dependent relationship between the two entities. In one of the largest studies to date that included 598 VLBW infants, forty-four whom developed at least stage II NEC, a hazard ratio of 5.99, p=0.001 was observed for those infants with severe anemia defined as hemoglobin <8 g/dL.39 Alternatively, in the recent Transfusion of Prematures (TOP) trial, a prospective study randomly assigning 1824 preterm neonates <29 weeks' gestation to higher versus lower hemoglobin thresholds, there was no difference observed in NEC rates for neonates that had a higher versus lower transfusion threshold, although this was not the primary outcome investigated.

Although the precise biological mechanism for NEC has not been definitively established, neonatal anemia can contribute to impaired oxygen delivery that may result in mesenteric vasculature. Underlying gastrointestinal hypoperfusion is thought to disturb the local microbiome, increase local production of pro-inflammatory mediators and exaggerate damage to the immature intestinal barrier. Our findings in FIGS. 18A-18F suggest that: 1) maternal anemia may be a novel risk factor for NEC and 2) there is a relationship at delivery between the degree of maternal and/or neonatal anemia and subsequent risk of NEC. Ultimately, further prospective investigations are needed to validate this relationship, assess whether transfusions at higher hemoglobin thresholds are protective and determine if there is a role for hormone replacement therapy (erythropoietin, darbepoetin alpha) in select populations at risk for complications secondary to severe anemia. Additionally, it is recognized that the number needed to screen and/or treat when evaluating maternal anemia is likely to be quite high given the relative rarity of a NEC diagnosis.

### Limitations

There are several limitations that must be considered when evaluating this investigation. First, we recognize that ICD coding as captured from the EHR does not always completely mimic or replicate clinical findings in patients. This is particularly true for categories such as diagnoses where clinical variability and interpretation can result in subjectivity. Additionally, although the overall prevalence of neonatal disease at our single institution site approximates prevalence across the United States, it is recognizes that these results may not be entirely generalizable across all institutions. However, such models are capable of predicting neonatal outcomes independent of institution. Moreover, clinical risk prediction models should recommend specific decisions that a clinician can employ as studies have shown that it is the recommendations that are likely to influence provider behavior. This investigation has been designed and intended as a first step in longitudinal risk prediction. Only through additional validation can our predictive models reasonably be used to recommend specific interventions or therapies.

Conclusion: The machine learning methodology employed herein has allowed the building of predictive models for neonatal outcomes and will potentially serve as an important resource for clinicians and researchers to examine independently. It has been observed that novel associations between various maternal and neonatal features and specific neonatal outcomes. The first longitudinal clinical risk prediction tool for various neonatal outcomes has been developed. A greater insight into the effect of the fetal environment has been gained and how it may contribute to risk for neonatal disease.

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

TABLE 1
List of maternal vitals and laboratory measurements

| | Unit of | Median [IQR] at | | % available at |
|---|---|---|---|---|
| Measurement | measure | delivery/birth | | delivery/birth |
| Vitals Measurements | | | | |
| Body weight | g | 2688 | (2400, 3040) | 81.7 |
| Body height | inches | 64 | (62, 66) | 76.1 |
| Body mass index (BMI) [Ratio] | kg/m² | 29.01 | (26.13, 32.82) | 76.8 |
| Body surface area | per m² | 1.85 | (1.74, 1.99) | 76.8 |
| Pulse rate | counts/min | 80 | (72, 88) | 97.4 |
| Respiratory rate | counts/min | 18 | (16, 18) | 99.7 |
| Body temperature | faraday | 98.2 | (97.9, 98.6) | 99.7 |
| Heart rate | counts/min | 82 | (72, 93) | 99.7 |
| Diastolic blood pressure | mmHg | 67 | (59, 75) | 99.7 |
| Systolic blood pressure | mmHg | 115 | (106, 126) | 99.7 |
| Oxygen saturation | % | 99 | (98, 100) | 89.6 |
| Laboratory Measurements | | | | |
| Alanine aminotransferase [Enzymatic | unit/l | 22 | (18, 30) | 32.2 |
| activity/volume] in Serum or Plasma | | | | |
| Albumin [Mass/volume] in Serum or Plasma | g/dl | 3 | (2.6, 3.5) | 14.8 |
| Alkaline phosphatase [Enzymatic | unit/l | 118 | (89, 155) | 14.7 |
| activity/volume] in Serum or Plasma | | | | |
| Anion gap in Serum or Plasma | mmol/l | 10 | (8, 12) | 16.5 |
| Aspartate aminotransferase [Enzymatic | unit/l | 19 | (14, 27) | 32.2 |
| activity/volume] in Serum or Plasma | | | | |
| Basophils [#/volume] in Blood by Automated | thousand | 0.03 | (0.02, 0.05) | 64.7 |
| count | per ml | | | |
| Basophils [#/volume] in Blood by Manual count | thousand | 0.03 | (0.02, 0.05) | 83.1 |
| | per ml | | | |
| Basophils/100 leukocytes in Blood | % | 0.3 | (0.2, 0.5) | 83.0 |
| Basophils/100 leukocytes in Blood by | % | 0.3 | (0.2, 0.5) | 64.8 |
| Automated count | | | | |
| Bicarbonate [Moles/volume] in Venous blood | mmol/l | 21.5 | (20, 22.9) | 30.2 |
| Bilirubin total [Mass/volume] in Serum or | mg/dl | 0.3 | (0.2, 0.4) | 14.9 |
| Plasma | | | | |
| Body surface area | per m² | 1.84 | (1.72, 1.97) | 73.8 |
| Calcium [Mass/volume] in Serum or Plasma | mg/dl | 8.8 | (8.5, 9.1) | 16.7 |
| Carbon dioxide, total [Moles/volume] in Serum | mmol/l | 23 | (21, 25) | 16.7 |
| or Plasma | | | | |
| Chloride [Moles/volume] in Serum or Plasma | mmol/l | 103 | (102, 105) | 16.7 |
| Creatinine [Mass/volume] in Serum or Plasma | mg/dl | 0.6 | (0.51, 0.71) | 32.8 |
| Eosinophils [#/volume] in Blood by Automated | thousand | 0.07 | (0.04, 0.11) | 92.9 |
| count | per ml | | | |
| Eosinophils/100 leukocytes in Blood by | % | 0.7 | (0.4, 1.1) | 93.0 |
| Automated count | | | | |
| Erythrocyte distribution width [Ratio] by | | 13.9 | (13.3, 14.7) | 97.0 |
| Automated count | | | | |
| Erythrocytes [#/volume] in Blood | million | 4.1 | (3.84, 4.36) | 87.8 |
| | per ml | | | |
| Erythrocytes [#/volume] in Blood by Automated | million | 4.04 | (3.73, 4.31) | 23.9 |
| count | per l | | | |
| Erythrocytes [#/volume] in Urine by Automated | million | 4.1 | (3.84, 4.36) | 87.7 |
| count | per ml | | | |
| Fasting glucose [Mass/volume] in Serum or | mg/dl | 80 | (75, 86) | 27.1 |
| Plasma | | | | |
| Globulin [Mass/volume] in Serum | g/dl | 3.6 | (3, 4.1) | 13.5 |
| Glomerular filtration rate in Serum, Plasma or | ml/min/ | 128 | (113, 141) | 17.2 |
| Blood | 1.73 m² | | | |
| Glomerular filtration rate in Serum, Plasma or | ml/min/ | 128 | (112, 141) | 17.0 |
| Blood by Creatinine-based formula (MDRD) | 1.73 m² | | | |
| Glucose [Mass/volume] in Serum or Plasma | mg/dl | 90 | (80, 104) | 31.6 |
| Hematocrit [Volume Fraction] of Blood by | % | 36.3 | (33.5, 38.6) | 98.6 |
| Automated count | | | | |
| Hemoglobin [Mass/volume] in Blood | mg/dl | 12.1 | (11.1, 12.9) | 98.1 |
| Hemoglobin A1c/Hemoglobin total in Blood | % | 5.2 | (4.9, 5.5) | 10.4 |
| Immature granulocytes [#/volume] in Blood by | thousand | 0.07 | (0.04, 0.11) | 34.3 |
| Automated count | per ml | | | |
| Immature granulocytes/100 leukocytes in Blood | % | 0.7 | (0.5, 1) | 34.3 |
| by Automated count | | | | |
| Input/Output | ml | 500 | (300, 700) | 98.6 |
| Leukocytes [#/volume] in Blood | thousand | 10.3 | (8.6, 12.8) | 87.8 |
| | per ml | | | |
| Leukocytes [#/volume] in Blood by Automated | thousand | 4.1 | (3.82, 4.38) | 45.6 |
| count | per ml | | | |
| Leukocytes [#/volume] in Unspecified specimen | thousand | 10.5 | (8.7, 13) | 67.9 |
| by Automated count | per ml | | | |
| Leukocytes [Presence] in Urine | thousand | 10.3 | (8.6, 12.8) | 87.7 |
| | per ml | | | |
| Lymphocytes [#/volume] in Blood by | thousand | 1.79 | (1.44, 2.2) | 93.2 |
| Automated count | per ml | | | |
| Lymphocytes/100 leukocytes in Blood by | % | 18.2 | (14.1, 22.4) | 93.4 |
| Automated count | | | | |
| MCH [Entitic mass] | pg | 30 | (28.3, 31.4) | 87.8 |
| MCH [Entitic mass] by Automated count | pg | 29.9 | (28.2, 31.3) | 67.9 |
| MCHC [Mass/volume] | g/dl | 33.3 | (32.7, 33.9) | 87.8 |
| MCHC [Mass/volume] by Automated count | g/dl | 33.2 | (32.6, 33.8) | 67.9 |
| MCV [Entitic volume] | fl | 89.9 | (86, 93.2) | 88.0 |
| MCV [Entitic volume] by Automated count | fl | 89.7 | (85.9, 93.1) | 67.9 |
| Mean blood pressure | mmHg | 84.33 | (77, 92) | 54.5 |
| Monocytes [#/volume] in Blood by Automated | thousand | 0.67 | (0.54, 0.83) | 93.2 |
| count | per ml | | | |
| Monocytes/100 leukocytes in Blood by | % | 6.7 | (5.5, 7.9) | 93.4 |
| Automated count | | | | |
| Neutrophils [#/volume] in Blood by Automated | thousand | 7.3 | (5.87, 9.32) | 93.4 |
| count | per ml | | | |
| Neutrophils/100 leukocytes in Blood | % | 73.2 | (68.3, 78.2) | 83.4 |
| Neutrophils/100 leukocytes in Blood by | % | 72.7 | (68, 77.7) | 64.7 |
| Automated count | | | | |
| Nucleated erythrocytes [#/volume] in Blood | % | 0 | (0, 0) | 34.8 |
| Nucleated erythrocytes/100 leukocytes [Ratio] | thousand | 0 | (0, 0) | 34.8 |
| in Body fluid | per ml | | | |
| Pain severity [Score] Visual analog score | | 1 | (0, 5) | 28.4 |
| pH of Urine by Test strip | | 6.5 | (6, 7) | 47.9 |
| Platelets [#/volume] in Blood | thousand | 200 | (166, 239) | 88.6 |
| | per ml | | | |
| Platelets [#/volume] in Blood by Automated | thousand | 201 | (166, 240) | 67.8 |
| count | per ml | | | |
| Potassium [Moles/volume] in Serum or Plasma | mmol/l | 3.9 | (3.6, 4.1) | 17.2 |
| Protein [Mass/volume] in Serum or Plasma | g/dl | 6.7 | (6.2, 7.2) | 14.7 |
| Protein [Mass/volume] in Urine | mg/dl | 9 | (6.5, 28) | 28.0 |
| Sodium [Moles/volume] in Blood | mmol/l | 137 | (135, 138) | 15.4 |
| Specific gravity of Urine | | 1.01 | (1.01, 1.02) | 43.8 |
| Specific gravity of Urine by Test strip | | 1.01 | (1.01, 1.02) | 26.0 |
| Urea nitrogen [Mass/volume] in Serum or | mg/dl | 9 | (7, 12) | 16.7 |
| Plasma | | | | |

TABLE 2
list of Observational Medical Outcomes Partnership (OMOP) Common Data Model
(CDM) concept IDs used to defined each of the 24 neonatal outcomes considered

| Concept ID | Concept name | outcome |
|---|---|---|
| 4048150 | Neonatal aspiration of milk and regurgitated food | MAS + Other |
| | | Aspiration |
| 4173178 | Neonatal aspiration of mucus | MAS + Other |
| | | Aspiration |
| 4153454 | Aspiration of liquor or mucus in newborn | MAS + Other |
| | | Aspiration |
| 4048457 | Aspiration of vomit in newborn | MAS + Other |
| | | Aspiration |
| 439934 | Meconium aspiration syndrome | MAS + Other |
| | | Aspiration |
| 437374 | Neonatal aspiration of meconium | MAS + Other |
| | | Aspiration |
| 4172995 | Neonatal aspiration of milk | MAS + Other |
| | | Aspiration |
| 433589 | Neonatal aspiration of amniotic fluid | MAS + Other |
| | | Aspiration |
| 434154 | Neonatal aspiration syndromes | MAS + Other |
| | | Aspiration |
| 45765391 | Chorea-athetoid cerebral palsy | CP |
| 442543 | Monoplegic cerebral palsy | CP |
| 4101736 | Hypotonic cerebral palsy | CP |
| 4043747 | Spastic cerebral palsy | CP |
| 4159737 | Paraplegic cerebral palsy | CP |
| 4043884 | Monoplegic cerebral palsy affecting lower limb | CP |
| 45771250 | Triplegic cerebral palsy | CP |
| 45773357 | Dystonic cerebral palsy | CP |
| 132617 | Diplegic cerebral palsy | CP |
| 44806793 | Spastic hemiplegic cerebral palsy | CP |
| 4173811 | Congenital quadriplegia | CP |
| 37396501 | Worster Drought syndrome | CP |
| 45765394 | Pentaplegic cerebral palsy | CP |
| 45765390 | Non-spastic cerebral palsy | CP |
| 4045844 | Dyskinetic cerebral palsy | CP |
| 44811521 | Bilateral spastic cerebral palsy | CP |
| 45765393 | Bilateral cerebral palsy | CP |
| 4195154 | Spastic tetraplegia with rigidity syndrome | CP |
| 4048800 | Dystonic/rigid cerebral palsy | CP |
| 4141403 | Cerebral palsy, not congenital or infantile, acute | CP |
| 44809963 | Choreo-athetotic cerebral palsy | CP |
| 4045842 | Monoplegic cerebral palsy affecting upper limb | CP |
| 762354 | Neuromuscular scoliosis of thoracolumbar spine | CP |
| | co-occurrent and due to cerebral palsy | |
| 37204364 | Severe microbrachycephaly, intellectual disability, | CP |
| | athetoid cerebral palsy syndrome | |
| 45765392 | Mixed cerebral palsy | CP |
| 762348 | Neuromuscular scoliosis of lumbar spine co- | CP |
| | occurrent and due to cerebral palsy | |
| 375525 | Athetoid cerebral palsy | CP |
| 134031 | Hemiplegic cerebral palsy | CP |
| 444022 | Tetraplegic cerebral palsy | CP |
| 4058438 | Double athetosis | CP |
| 4150300 | Ataxic cerebral palsy | CP |
| 4134120 | Cerebral palsy | CP |
| 45771249 | Choreic cerebral palsy | CP |
| 4236182 | Interstitial pulmonary fibrosis of prematurity | BPD |
| 42600161 | Pulmonary nodular fibroplasia | BPD |
| 4263344 | Pulmonary fibroplasia | BPD |
| 4283942 | Bronchopulmonary dysplasia of newborn | BPD |
| 4201423 | Wilson-Mikity syndrome | BPD |
| 4263343 | Perinatal pulmonary fibroplasia | BPD |
| 313023 | Chronic respiratory disease in perinatal period | BPD |
| 4079973 | Perinatal subependymal hemorrhage | IVH |
| 42535103 | Neonatal non-traumatic intraventricular | IVH |
| | hemorrhage | |
| 36716544 | Fetal or neonatal non-traumatic intraventricular | IVH |
| | hemorrhage | |
| 4048279 | Intraventricular hemorrhage due to birth injury | IVH |
| 4048278 | Intraventricular (nontraumatic) hemorrhage, grade | IVH |
| | 2, of fetus and newborn | |
| 436519 | Perinatal intraventricular hemorrhage | IVH |
| 4144154 | Non-traumatic intracerebral ventricular | IVH |
| | hemorrhage | |
| 434155 | Intraventricular (nontraumatic) hemorrhage, grade | IVH |
| | 3, of fetus and newborn | |
| 4110185 | Intracerebral hemorrhage, intraventricular | IVH |
| 4171123 | Perinatal subependymal hemorrhage with | IVH |
| | intraventricular and intracerebral extension | |
| 4048277 | Intraventricular (nontraumatic) hemorrhage, grade | IVH |
| | 1, of fetus and newborn | |
| 4173332 | Perinatal subependymal hemorrhage with | IVH |
| | intraventricular extension | |
| 36716627 | Traumatic intraventricular hemorrhage | IVH |
| 4079972 | Intraventricular hemorrhage of prematurity | IVH |
| 4180743 | Intraventricular hemorrhage of fetus | IVH |
| 36716543 | Fetal or neonatal intraventricular non-traumatic | IVH |
| | hemorrhage grade 4 | |
| 443752 | Ventricular hemorrhage | IVH |
| 37394466 | Intraventricular (nontraumatic) haemorrhage, | IVH |
| | grade 4, of fetus and newborn | |
| 37311911 | Necrotizing enterocolitis of newborn, stage 1B | NEC |
| 4308227 | Neonatal necrotizing enterocolitis | NEC |
| 201957 | Necrotizing enterocolitis in fetus OR newborn | NEC |
| 37311908 | Necrotizing enterocolitis of newborn, stage 3A | NEC |
| 37311912 | Necrotizing enterocolitis of newborn, stage 1A | NEC |
| 37311909 | Necrotizing enterocolitis of newborn, stage 2B | NEC |
| 37311910 | Necrotizing enterocolitis of newborn, stage 2A | NEC |
| 37311907 | Necrotizing enterocolitis of newborn, Stage 3B | NEC |
| 4287783 | Perinatal necrotizing enterocolitis | NEC |
| 43021583 | Patent arterial duct with normal origin and | PDA |
| | insertion | |
| 37205075 | Pulmonary valve agenesis, intact ventricular | PDA |
| | septum, persistent ductus arteriosus syndrome | |
| 4053893 | Patent ductus arteriosus with left-to-right shunt | PDA |
| 4109328 | Delayed closure of patent arterial duct | PDA |
| 37204212 | Multisystemic smooth muscle dysfunction | PDA |
| | syndrome | |
| 315922 | Patent ductus arteriosus | PDA |
| 4053656 | Patent ductus arteriosus with right-to-left shunt | PDA |
| 372435 | Periventricular leukomalacia | PVL |
| 4071867 | Neonatal cerebral leukomalacia | PVL |
| 45768986 | Acute respiratory distress in newborn with | RDS |
| | surfactant disorder | |
| 45772947 | Acute respiratory distress in newborn | RDS |
| 258866 | Respiratory distress syndrome in the newborn | RDS |
| 37207968 | Bilateral retinopathy of prematurity of eyes stage 0 | ROP |
| 36684751 | Bilateral retinopathy of prematurity of eyes stage 3 - | ROP |
| | ridge with extraretinal fibrovascular proliferation | |
| 443520 | Retinopathy of prematurity stage 5 - total retinal | ROP |
| | detachment | |
| 36684752 | Bilateral retinopathy of prematurity of eyes stage 2 - | ROP |
| | intraretinal ridge | |
| 373766 | Retinopathy of prematurity | ROP |
| 36684624 | Retinopathy of prematurity of right eye | ROP |
| 443519 | Retinopathy of prematurity stage 4 - subtotal | ROP |
| | retinal detachment | |
| 375251 | Retinopathy of prematurity stage 2 - intraretinal | ROP |
| | ridge | |
| 36684621 | Retinopathy of prematurity of right eye stage 3 - | ROP |
| | ridge with extraretinal fibrovascular proliferation | |
| 36684754 | Bilateral retinopathy of prematurity | ROP |
| 36684622 | Retinopathy of prematurity of right eye stage 2 - | ROP |
| | intraretinal ridge | |
| 36684753 | Bilateral retinopathy of prematurity of eyes stage | ROP |
| | 1 - demarcation line | |
| 36684685 | Retinopathy of prematurity of left eye stage 2 - | ROP |
| | intraretinal ridge | |
| 36684687 | Retinopathy of prematurity of left eye | ROP |
| 36684684 | Retinopathy of prematurity of left eye stage 3 - | ROP |
| | ridge with extraretinal fibrovascular proliferation | |
| 36684686 | Retinopathy of prematurity of left eye stage 1 - | ROP |
| | demarcation line | |
| 375250 | Retinopathy of prematurity stage 1 - demarcation | ROP |
| | line | |
| 36684623 | Retinopathy of prematurity of right eye stage 1 - | ROP |
| | demarcation line | |
| 379009 | Retinopathy of prematurity stage 3 - ridge with | ROP |
| | extraretinal fibrovascular proliferation | |
| 4339722 | Neonatal anemia | Anemia of prematurity |
| 4079852 | Physiological anemia of infancy | Anemia of prematurity |
| 4173191 | Late anemia of newborn | Anemia of prematurity |
| 36713168 | Hemolytic disease of newborn co-occurrent and | Anemia of prematurity |
| | due to ABO immunization | |
| 4071073 | Late anemia of newborn due to isoimmunization | Anemia of prematurity |
| 36674478 | Neonatal autoimmune hemolytic anemia | Anemia of prematurity |
| 432452 | Anemia of prematurity | Anemia of prematurity |
| 4173191 | Late anemia of newborn | Anemia of prematurity |
| 4071073 | Late anemia of newborn due to isoimmunization | Anemia of prematurity |
| 133594 | Bacterial sepsis of newborn | Sepsis |
| 4048275 | Sepsis of newborn due to anaerobes | Sepsis |
| 46270041 | Sepsis of newborn due to group B Streptococcus | Sepsis |
| 36715567 | Neonatal sepsis caused by Malassezia | Sepsis |
| 761851 | Neonatal sepsis caused by Staphylococcus | Sepsis |
| 35622880 | Early-onset neonatal sepsis | Sepsis |
| 42536689 | Sepsis of neonate caused by Streptococcus | Sepsis |
| | pyogenes | |
| 4071727 | Sepsis of newborn due to Escherichia coli | Sepsis |
| 4048594 | Sepsis of newborn due to Staphylococcus aureus | Sepsis |
| 35622881 | Late-onset neonatal sepsis | Sepsis |
| 4071063 | Sepsis of the newborn | Sepsis |
| 761852 | Neonatal sepsis caused by Streptococcus | Sepsis |
| 763027 | Sepsis of newborn due to Streptococcus agalactiae | Sepsis |
| 4071740 | Neonatal jaundice with Crigler-Najjar syndrome | Hyperbilirubinemia |
| 4067525 | Fetal OR neonatal jaundice from polycythemia | Hyperbilirubinemia |
| 4170445 | Neonatal jaundice due to deficiency of enzyme | Hyperbilirubinemia |
| | system for bilirubin conjugation | |
| 4071737 | Perinatal jaundice from bleeding | Hyperbilirubinemia |
| 4071736 | Perinatal jaundice from polycythemia | Hyperbilirubinemia |
| 4096143 | Fetal OR neonatal jaundice from swallowed | Hyperbilirubinemia |
| | maternal blood | |
| 4071080 | Neonatal jaundice with porphyria | Hyperbilirubinemia |
| 4251487 | Perinatal jaundice due to inspissated bile | Hyperbilirubinemia |
| | syndrome | |
| 440847 | Neonatal jaundice associated with preterm | Hyperbilirubinemia |
| | delivery | |
| 4071741 | Perinatal jaundice due to congenital obstruction of | Hyperbilirubinemia |
| | bile duct | |
| 4048294 | Neonatal jaundice with Rotor's syndrome | Hyperbilirubinemia |
| 4239658 | Neonatal jaundice due to delayed conjugation | Hyperbilirubinemia |
| | from delayed development of conjugating system | |
| 4048290 | Neonatal jaundice due to glucose-6-phosphate | Hyperbilirubinemia |
| | dehydrogenase deficiency | |
| 4071083 | Perinatal jaundice due to galactosemia | Hyperbilirubinemia |
| 4048614 | Neonatal jaundice with Gilbert's syndrome | Hyperbilirubinemia |
| 435656 | Neonatal jaundice | Hyperbilirubinemia |
| 4071079 | Neonatal jaundice with congenital hypothyroidism | Hyperbilirubinemia |
| 4048293 | Delayed conjugation causing neonatal jaundice | Hyperbilirubinemia |
| | associated with another disorder | |
| 4048610 | Perinatal jaundice from swallowed maternal blood | Hyperbilirubinemia |
| 4230351 | Fetal OR neonatal jaundice from infection | Hyperbilirubinemia |
| 4221399 | Neonatal jaundice due to delayed conjugation | Hyperbilirubinemia |
| | from breast milk inhibitor | |
| 4048613 | Neonatal jaundice with Dubin-Johnson syndrome | Hyperbilirubinemia |
| 4328890 | Fetal OR neonatal jaundice from drugs AND/OR | Hyperbilirubinemia |
| | toxins transmitted from mother | |
| 4071735 | Perinatal jaundice from bruising | Hyperbilirubinemia |
| 4071743 | Perinatal jaundice due to cystic fibrosis | Hyperbilirubinemia |
| 4165508 | Lucey-Driscoll syndrome | Hyperbilirubinemia |
| 4071076 | Perinatal jaundice from maternal transmission of | Hyperbilirubinemia |
| | drug or toxin | |
| 4171095 | Prolonged newborn physiological jaundice | Hyperbilirubinemia |
| 439137 | Neonatal jaundice due to delayed conjugation | Hyperbilirubinemia |
| 4173180 | Newborn physiological jaundice | Hyperbilirubinemia |
| 442255 | Intestinal obstruction by inspissated milk in | Neonatal |
| | newborn | gastroesophageal |
| | | reflux |
| 42536732 | Neonatal intestinal perforation due to in utero | Neonatal |
| | intestinal volvulus | gastroesophageal |
| | | reflux |
| 42536730 | Neonatal intestinal perforation co-occurrent and | Neonatal |
| | due to intestinal atresia | gastroesophageal |
| | | reflux |
| 4172869 | Peptic ulcer of newborn | Neonatal |
| | | gastroesophageal |
| | | reflux |
| 37116437 | Neonatal obstruction of intestine | Neonatal |
| | | gastroesophageal |
| | | reflux |
| 42536564 | Neonatal perforation of intestine caused by drug | Neonatal |
| | | gastroesophageal |
| | | reflux |
| 42536733 | Neonatal isolated ileal perforation | Neonatal |
| | | gastroesophageal |
| | | reflux |
| 4071070 | Neonatal hematemesis | Neonatal |
| | | gastroesophageal |
| | | reflux |
| 4319461 | Paralytic ileus of the newborn | Neonatal |
| | | gastroesophageal |
| | | reflux |
| 36712969 | Neonatal gastroesophageal reflux | Neonatal |
| | | gastroesophageal |
| | | reflux |
| 4048286 | Neonatal rectal hemorrhage | Neonatal |
| | | gastroesophageal |
| | | reflux |
| 42536731 | Neonatal intestinal perforation with congenital | Neonatal |
| | intestinal stenosis | gastroesophageal |
| | | reflux |
| 36676688 | Neonatal inflammatory skin and bowel disease | Neonatal |
| | | gastroesophageal |
| | | reflux |
| 4180181 | Neonatal gastrointestinal disorder | Neonatal |
| | | gastroesophageal |
| | | reflux |
| 4318858 | Spastic ileus of the newborn | Neonatal |
| | | gastroesophageal |
| | | reflux |
| 36715839 | Neonatal eosinophilic esophagitis | Neonatal |
| | | gastroesophageal |
| | | reflux |
| 4172870 | Gastritis of newborn | Neonatal |
| | | gastroesophageal |
| | | reflux |
| 36717488 | Neonatal esophagitis | Neonatal |
| | | gastroesophageal |
| | | reflux |
| 42539039 | Neonatal intestinal perforation with in utero | Neonatal |
| | intraluminal obstruction | gastroesophageal |
| | | reflux |
| 37109016 | Neonatal gastrointestinal hemorrhage | Neonatal |
| | | gastroesophageal |
| | | reflux |
| 42536729 | Neonatal malabsorption with gastrointestinal | Neonatal |
| | hormone-secreting endocrine tumor | gastroesophageal |
| | | reflux |
| 4316375 | Neonatal respiratory alkalosis | Respiratory failure |
| 4051337 | Neonatal pneumonia | Respiratory failure |
| 318856 | Neonatal respiratory arrest | Respiratory failure |
| 4080883 | Neonatal aspiration pneumonia | Respiratory failure |
| 36716747 | Acquired vocal cord paralysis in newborn | Respiratory failure |
| 36716745 | Neonatal hypotonia of hypopharynx | Respiratory failure |
| 252305 | Obstructive apnea of newborn | Respiratory failure |
| 4147117 | Perinatal respiratory distress | Respiratory failure |
| 4079694 | Perinatal pneumoperitoneum | Respiratory failure |
| 4181199 | Neonatal respiratory system disorder | Respiratory failure |
| 4172872 | Chronic pulmonary insufficiency of prematurity | Respiratory failure |
| 36716886 | Neonatal mass of hypopharynx | Respiratory failure |
| 36716750 | Neonatal epistaxis | Respiratory failure |
| 4079848 | Apnea of prematurity | Respiratory failure |
| 4262580 | Primary sleep apnea of newborn | Respiratory failure |
| 42539560 | Mixed neonatal apnea | Respiratory failure |
| 36716743 | Central neonatal apnea | Respiratory failure |
| 4318857 | Neonatal respiratory depression | Respiratory failure |
| 37116463 | Acquired neonatal pulmonary cysts | Respiratory failure |
| 37108745 | Neonatal pneumomediastinum | Respiratory failure |
| 4173177 | Respiratory insufficiency syndrome of newborn | Respiratory failure |
| 258564 | Perinatal interstitial emphysema | Respiratory failure |
| 36717571 | Neonatal traumatic hemorrhage of trachea | Respiratory failure |
| | following procedure on lower respiratory tract | |
| 4317960 | Neonatal respiratory failure | Respiratory failure |
| 4172996 | Neonatal tracheal perforation | Respiratory failure |
| 4110550 | Neonatal cardiorespiratory arrest | Respiratory failure |
| 4171093 | Neonatal pulmonary air leak | Respiratory failure |
| 4070651 | Neonatal candidiasis of lung | Respiratory failure |
| 4318553 | Respiratory tract hemorrhage of the newborn | Respiratory failure |
| 4316374 | Neonatal respiratory acidosis | Respiratory failure |
| 4051333 | Neonatal chlamydial pneumonia | Respiratory failure |
| 37311892 | Primary central sleep apnea of prematurity | Respiratory failure |
| 4173330 | Acquired subglottic stenosis in newborn | Respiratory failure |
| 4210115 | Neonatal tracheobronchial hemorrhage | Respiratory failure |
| 4171094 | Prolonged apnea of newborn | Respiratory failure |
| 42536748 | Infection causing tracheitis in neonate | Respiratory failure |
| 42536753 | Tracheo-bronchial malacia in neonate | Respiratory failure |
| 36716741 | Respiratory instability of prematurity | Respiratory failure |
| 36716744 | Apnea of newborn due to neurological injury | Respiratory failure |
| 4149586 | Perinatal massive pulmonary hemorrhage | Pulmonary hemorrhage |
| 42573131 | Bleeder syndrome | Pulmonary hemorrhage |
| 256036 | Hemorrhagic varicella pneumonitis | Pulmonary hemorrhage |
| 195289 | Goodpasture's syndrome | Pulmonary hemorrhage |
| 257375 | Neonatal pulmonary hemorrhage | Pulmonary hemorrhage |
| 42536566 | Perinatal hemorrhage of lung due to traumatic | Pulmonary hemorrhage |
| | injury | |
| 4111119 | Hemorrhagic bronchopneumonia | Pulmonary hemorrhage |
| 4051335 | Hemorrhagic pneumonia | Pulmonary hemorrhage |
| 761075 | Acute idiopathic neonatal pulmonary hemorrhage | Pulmonary hemorrhage |
| 4171119 | Hemorrhagic pulmonary edema | Pulmonary hemorrhage |
| 43021073 | Perinatal pulmonary hemorrhage | Pulmonary hemorrhage |
| 4301606 | Pulmonary hemorrhage | Pulmonary hemorrhage |
| 42573132 | Exercise-induced pulmonary hemorrhage | Pulmonary hemorrhage |
| 4071717 | Perinatal lung intra-alveolar hemorrhage | Pulmonary hemorrhage |
| 44783620 | Heritable pulmonary arterial hypertension due to | Pulmonary HTN |
| | ALK1 or endoglin mutation | |
| 44783619 | Heritable pulmonary arterial hypertension due to | Pulmonary HTN |
| | BMPR2 mutation | |
| 40493243 | Eisenmenger's syndrome | Pulmonary HTN |
| 44783622 | Pulmonary arterial hypertension associated with | Pulmonary HTN |
| | connective tissue disease | |
| 40482858 | Pulmonary arterial hypertension associated with | Pulmonary HTN |
| | portal hypertension | |
| 4124831 | Sporadic primary pulmonary hypertension | Pulmonary HTN |
| 4013643 | Pulmonary arterial hypertension | Pulmonary HTN |
| 44782561 | Pulmonary arterial hypertension induced by toxin | Pulmonary HTN |
| 44783625 | Pulmonary arterial hypertension associated with | Pulmonary HTN |
| | schistosomiasis | |
| 44782562 | Pulmonary arterial hypertension associated with | Pulmonary HTN |
| | congenital systemic-to-pulmonary shunt | |
| 44783621 | Associated pulmonary arterial hypertension | Pulmonary HTN |
| 4121462 | Persistent pulmonary hypertension of the newborn | Pulmonary HTN |
| 44783623 | Pulmonary arterial hypertension associated with | Pulmonary HTN |
| | HIV infection | |
| 44783624 | Pulmonary arterial hypertension associated with | Pulmonary HTN |
| | congenital heart disease | |
| 4119611 | Familial primary pulmonary hypertension | Pulmonary HTN |
| 4121620 | Pulmonary arterial hypertension induced by drug | Pulmonary HTN |
| 44783618 | Heritable pulmonary arterial hypertension | Pulmonary HTN |
| 44783626 | Pulmonary arterial hypertension associated with | Pulmonary HTN |
| | chronic hemolytic anemia | |
| 44782560 | Idiopathic pulmonary arterial hypertension | Pulmonary HTN |
| 36715093 | Braddock syndrome | Pulmonary HTN |
| 4043411 | Benign neonatal familial convulsions | Seizures |
| 4046209 | Benign non-familial neonatal convulsions | Seizures |
| 762706 | Benign familial neonatal seizures, non-refractory | Seizures |
| 4171110 | Fifth day fits | Seizures |
| 37399364 | Folinic acid responsive seizure syndrome | Seizures |
| 4159149 | Seizures complicating intracranial hemorrhage in | Seizures |
| | the newborn | |
| 762705 | Benign familial neonatal seizures, refractory | Seizures |
| 37395921 | ICCA syndrome | Seizures |
| 762709 | Seizures in the newborn, non-refractory | Seizures |
| 762579 | Seizures in the newborn, refractory | Seizures |
| 380533 | Convulsions in the newborn | Seizures |
| 4089691 | Familial neonatal seizures | Seizures |
| 4186827 | Seizures complicating infection in the newborn | Seizures |
| 36675039 | Severe neonatal onset encephalopathy with | Seizures |
| | microcephaly | |
| 4244383 | Benign neonatal convulsions | Seizures |
| 46273607 | MECP2-related severe neonatal encephalopathy | Other CNS disorders |
| 42535008 | Mild hypoxic ischemic encephalopathy of | Other CNS disorders |
| | newborn | |
| 42535007 | Moderate hypoxic ischemic encephalopathy of | Other CNS disorders |
| | newborn | |
| 4061270 | Neonatal agitation | Other CNS disorders |
| 444292 | Cerebral depression in newborn | Other CNS disorders |
| 4318859 | Neonatal encephalopathy | Other CNS disorders |
| 4200079 | Head lag in the newborn | Other CNS disorders |
| 36714076 | Symmetrical thalamic calcification | Other CNS disorders |
| 4290019 | Central nervous system dysfunction in newborn | Other CNS disorders |
| 4182388 | Lethal neonatal spasticity | Other CNS disorders |
| 377980 | Cerebral irritability in newborn | Other CNS disorders |
| 36674814 | Neonatal brainstem dysfunction | Other CNS disorders |
| 42535006 | Severe hypoxic ischemic encephalopathy of | Other CNS disorders |
| | newborn | |
| 372444 | Coma in the newborn | Other CNS disorders |
| 4082314 | Postnatal hypoxic encephalopathy | Other CNS disorders |
| 4318860 | Drowsiness of the newborn | Other CNS disorders |
| 4319463 | Neonatal hypokinesia | Other CNS disorders |
| 4079556 | Neonatal asphyxial encephalopathy | Other CNS disorders |
| 442631 | Abnormal cerebral signs in the newborn | Other CNS disorders |
| 42535380 | Hypoxic ischemic encephalopathy due to birth | Other CNS disorders |
| | trauma | |
| 4278842 | Perinatal pulmonary collapse | Atelectasis |
| 4243494 | Perinatal secondary atelectasis | Atelectasis |
| 260212 | Perinatal atelectasis | Atelectasis |
| 258554 | Primary atelectasis, in perinatal period | Atelectasis |
| 4006329 | Perinatal partial atelectasis | Atelectasis |
| 4300236 | Neonatal systemic candidiasis | Candidiasis |
| 440840 | Neonatal candidiasis | Candidiasis |
| 4070650 | Neonatal candidiasis of intestine | Candidiasis |
| 42538263 | Neonatal oral candidiasis | Candidiasis |
| 36717505 | Neonatal mucocutaneous infection caused by | Candidiasis |
| | Candida | |
| 4070651 | Neonatal candidiasis of lung | Candidiasis |
| 4070648 | Neonatal candidiasis of perineum | Candidiasis |
| 4173170 | Neonatal dysrhythmia | Cardiovascular |
| | | instability |
| 37395937 | Idiopathic neonatal atrial flutter | Cardiovascular |
| | | instability |
| 4106274 | Neonatal cardiac arrest | Cardiovascular |
| | | instability |
| 4110550 | Neonatal cardiorespiratory arrest | Cardiovascular |
| | | instability |
| 443522 | Neonatal bradycardia | Cardiovascular |
| | | instability |
| 443523 | Neonatal tachycardia | Cardiovascular |
| | | instability |
| 42537678 | Neonatal polycythemia due to placental | Polycythemia |
| | insufficiency | |
| 36716549 | Polycythemia neonatorum following blood | Polycythemia |
| | transfusion | |
| 4305235 | Polycythemia due to donor twin transfusion | Polycythemia |
| 42537679 | Neonatal polycythemia due to intra-uterine growth | Polycythemia |
| | retardation | |
| 439140 | Neonatal polycythemia | Polycythemia |
| 4297988 | Polycythemia due to maternal-fetal transfusion | Polycythemia |
| 36716548 | Polycythemia neonatorum due to inherited | Polycythemia |
| | disorder of erythropoietin production | |
| 36716748 | Neonatal cardiac failure due to decreased left | Cardiac failure |
| | ventricular output | |
| 4172864 | Neonatal cardiac failure | Cardiac failure |
| 37110330 | Neonatal cardiac failure due to pulmonary | Cardiac failure |
| | overperfusion | |

TABLE 3
Prevalence, AUPRC, AUPRC compared to a random classifier and AUC of
the AI model to predict the 24 neonatal outcomes, stratified by pre-
and full-term (gestational week at delivery <37 or ≥37 weeks)

| | Pre-term newborns (n = 3,936) | | | | Full-term newborns (n = 27,998) | | | |
|---|---|---|---|---|---|---|---|---|
| | Prevalence | | AUPRC | | Prevalence | | AUPRC | |
| Outcome | (%) | AUPRC | vs RC | AUC | (%) | AUPRC | vs RC | AUC |
| RDS | 37.0 | 0.776 | 2.09 | 0.836 | 3.1 | 0.076 | 2.42 | 0.662 |
| IVH | 5.1 | 0.183 | 3.56 | 0.846 | 0.2 | 0.048 | 21.82 | 0.878 |
| NEC | 1.8 | 0.105 | 5.71 | 0.851 | 0.04 | 0.045 | 115.73 | 0.869 |
| ROP | 15.1 | 0.699 | 4.64 | 0.918 | 0.02 | 0.007 | 32.01 | 0.663 |
| BPD | 7.1 | 0.507 | 7.19 | 0.934 | 0.04 | 0.019 | 43.85 | 0.891 |
| PDA | 11.1 | 0.439 | 3.96 | 0.862 | 2.0 | 0.363 | 18.43 | 0.823 |
| PVL | 0.6 | 0.052 | 8.57 | 0.884 | 0.0 | n/a | | n/a |
| Sepsis | 8.0 | 0.291 | 3.64 | 0.810 | 1.0 | 0.042 | 4.15 | 0.709 |
| Pulmonary hem. | 1.0 | 0.046 | 4.67 | 0.880 | 0.03 | 0.010 | 30.87 | 0.943 |
| CP | 1.0 | 0.030 | 3.13 | 0.798 | 0.1 | 0.027 | 24.28 | 0.838 |
| Pulmonary HTN | 1.6 | 0.098 | 5.96 | 0.883 | 0.3 | 0.074 | 22.66 | 0.847 |
| Hyperbilirubinemia | 71.9 | 0.862 | 1.20 | 0.728 | 43.8 | 0.525 | 1.20 | 0.606 |
| Death | 3.8 | 0.412 | 10.70 | 0.937 | 0.2 | 0.062 | 34.23 | 0.933 |
| MAS | 1.2 | 0.025 | 2.08 | 0.669 | 1.1 | 0.023 | 2.08 | 0.644 |
| Atelectasis | 4.4 | 0.270 | 6.11 | 0.868 | 0.5 | 0.291 | 56.24 | 0.881 |
| Candidiasis | 1.6 | 0.044 | 2.71 | 0.705 | 0.5 | 0.009 | 1.77 | 0.627 |
| Cardiac failure | 0.5 | 0.024 | 4.54 | 0.868 | 0.1 | 0.022 | 26.59 | 0.944 |
| Cardiovascular | 31.4 | 0.559 | 1.78 | 0.772 | 2.2 | 0.077 | 3.49 | 0.683 |
| Instability | | | | | | | | |
| Other CNS disorder | 0.4 | 0.007 | 1.96 | 0.659 | 0.1 | 0.007 | 5.35 | 0.769 |
| Neonatal | | | | | | | | |
| Gastroesophageal | 2.6 | 0.059 | 2.33 | 0.738 | 0.4 | 0.022 | 5.44 | 0.656 |
| Reflux | | | | | | | | |
| Respiratory failure | 8.4 | 0.217 | 2.57 | 0.756 | 2.0 | 0.118 | 5.94 | 0.704 |
| Polycythemia | 1.5 | 0.019 | 1.31 | 0.593 | 0.3 | 0.012 | 4.12 | 0.658 |
| Seizures | 1.3 | 0.043 | 3.25 | 0.777 | 0.2 | 0.021 | 8.58 | 0.781 |
| Anemia of | 20.5 | 0.724 | 3.53 | 0.902 | 0.0 | n/a | n/a | n/a |
| prematurity | | | | | | | | |

TABLE 4
Summary statistics of maternal/newborn
characteristics and neonatal outcomes.

| | n (%) or | |
|---|---|---|
| | mean (±SD) | |
| Maternal age at delivery | 32.8 | (±5.6) |
| Maternal Race/Ethnicity | | |
| Asian | 8,192 | (25.3%) |
| Black or African American | 695 | (2.1%) |
| Native Hawaiian | 642 | (2.0%) |
| White | 10,707 | (33.1%) |
| Hispanic/Other | 10,025 | (31.0%) |
| Decline to state | 641 | (2.0%) |
| Unknown | 1,452 | (4.5%) |
| Newborn Sex | | |
| Male | 16,710 | (51.6%) |
| Female | 15,400 | (47.6%) |
| Unknown | 244 | (0.8%) |
| Newborn birthweight [g] | 3,128 | (±525) |
| Gestational age at delivery [weeks] | 38.7 | (±2.1) |
| Gestational age <37 weeks | 3,639 | (11.5%) |
| Number of codes in maternal medical | 217 | (±236) |
| history up to delivery/birth | | |
| Number of codes in newborn's medical | 49 | (±73) |
| history up to two months after birth | | |
| Neonatal outcomes | | |
| RDS | 2,248 | (6.9%) |
| IVH | 249 | (0.8%) |
| NEC | 78 | (0.2%) |
| ROP | 554 | (1.7%) |
| BPD | 270 | (0.8%) |
| PDA | 965 | (3.0%) |
| PVL | 24 | (0.07%) |
| Sepsis | 579 | (1.8%) |
| Pulmonary hemorrhage | 46 | (0.1%) |
| CP | 66 | (0.2%) |
| Pulmonary HTN | 154 | (0.5%) |
| Hyperbilirubinemia | 15,015 | (46.4%) |
| Death | 230 | (0.7%) |
| MAS and other aspiration | 357 | (1.1%) |
| Atelectasis | 314 | (1.0%) |
| Candidiasis | 201 | (0.6%) |
| Cardiac failure | 42 | (0.1%) |
| Cardiovascular instability | 1,761 | (5.4%) |
| Other CNS disorder | 52 | (0.2%) |
| Neonatal gastroesophageal reflux | 206 | (0.6%) |
| Respiratory failure | 873 | (2.7%) |
| Polycythemia | 136 | (0.4%) |
| Seizures | 116 | (0.4%) |
| Anemia of prematurity | 750 | (2.3%) |

Note:
gestational age at delivery and newborn birthweight were missing for 713 and 463 newborns, respectively.
RDS: respiratory distress syndrome,
IVH: intraventricular hemorrhage,
NEC: necrotizing enterocolitis,
ROP: retinopathy of prematurity,
BPD: bronchopulmonary dysplasia,
PDA: patent ductus arteriosus,
PVL: periventricular leukomalacia,
CP: cerebral palsy,
HTN: hypertension,
MAS: meconium aspiration syndrome,
CNS: central nervous system

TABLE 5
Temporal dataset shifting experiment. Prevalence, AUPRC, AUPRC compared to a random
classifier and AUC of the original AI model (2014-2020) and in newborns born in
2019 and 2020 from the AI model trained on newborns born between 2014 and 2018.

| | | | | | | | AUPRC | | |
|---|---|---|---|---|---|---|---|---|---|
| | n (%) | | | | | | 2014- | | |
| | 2014-2020 | | 2019 | | 2020 | | 2020 | 2019 | 2020 |
| | (n = | | n = | | (n = | | (n = | (n = | (n = |
| | 32,350) | | (5,852) | | 4,397) | | 32,350) | 5,852) | 4,397) |
| RDS | 2,248 | (6.9%) | 391 | (6.7%) | 267 | (6.1%) | 0.541 | 0.461 | 0.474 |
| IVH | 249 | (0.8%) | 40 | (0.7%) | 32 | (0.7%) | 0.157 | 0.179 | 0.259 |
| NEC | 78 | (0.2%) | 17 | (0.3%) | 17 | (0.4%) | 0.096 | 0.128 | 0.174 |
| ROP | 554 | (1.7%) | 82 | (1.4%) | 60 | (1.4%) | 0.690 | 0.601 | 0.568 |
| BPD | 270 | (0.8%) | 35 | (0.6%) | 34 | (0.8%) | 0.487 | 0.486 | 0.515 |
| PDA | 965 | (3.0%) | 136 | (2.3%) | 112 | (2.5%) | 0.394 | 0.367 | 0.395 |
| PVL | 24 | (0.1%) | 7 | (0.1%) | 2 | (0.0%) | 0.048 | 0.065 | 0.031 |
| Sepsis | 579 | (1.8%) | 62 | (1.1%) | 45 | (1.0%) | 0.179 | 0.127 | 0.139 |
| Pulmonary hem. | 46 | (0.1%) | 10 | (0.2%) | 4 | (0.1%) | 0.040 | 0.099 | 0.014 |
| CP | 66 | (0.2%) | 6 | (0.1%) | 0 | (0.0%) | 0.025 | 0.023 | |
| Pulmonary HTN | 154 | (0.5%) | 22 | (0.4%) | 15 | (0.3%) | 0.082 | 0.040 | 0.039 |
| Hyperbilirubinemia | 15,014 | (46.4%) | 2,414 | (41.3%) | 1,679 | (38.2%) | 0.628 | 0.545 | 0.513 |
| Death | 230 | (0.7%) | 35 | (0.6%) | 26 | (0.6%) | 0.298 | 0.134 | 0.254 |
| MAS | 357 | (1.1%) | 65 | (1.1%) | 33 | (0.8%) | 0.021 | 0.015 | 0.016 |
| Atelectasis | 314 | (1.0%) | 20 | (0.3%) | 24 | (0.5%) | 0.286 | 0.055 | 0.089 |
| Candidiasis | 201 | (0.6%) | 39 | (0.7%) | 27 | (0.6%) | 0.020 | 0.011 | 0.029 |
| Cardiac failure | 42 | (0.1%) | 7 | (0.1%) | 5 | (0.1%) | 0.022 | 0.024 | 0.040 |
| Cardiovascular | 1,761 | (5.4%) | 301 | (5.1%) | 221 | (5.0%) | 0.422 | 0.359 | 0.275 |
| Instability | | | | | | | | | |
| Other CNS | 52 | (0.2%) | 9 | (0.2%) | 3 | (0.1%) | 0.007 | 0.004 | 0.013 |
| disorder | | | | | | | | | |
| Neonatal | 206 | (0.6%) | 46 | (0.8%) | 40 | (0.9%) | 0.039 | 0.037 | 0.044 |
| Gastroesophageal | | | | | | | | | |
| Reflux | | | | | | | | | |
| Respiratory failure | 873 | (2.7%) | 149 | (2.5%) | 114 | (2.6%) | 0.153 | 0.132 | 0.233 |
| Polycythemia | 136 | (0.4%) | 10 | (0.2%) | 9 | (0.2%) | 0.014 | 0.005 | 0.008 |
| Seizures | 116 | (0.4%) | 17 | (0.3%) | 15 | (0.3%) | 0.030 | 0.038 | 0.082 |
| Anemia of | 750 | (2.3%) | 121 | (2.1%) | 75 | (1.7%) | 0.717 | 0.648 | 0.654 |
| prematurity | | | | | | | | | |

| | AUPRC vs RC | | | AUC | | |
|---|---|---|---|---|---|---|
| | 2014- | | | 2014- | | |
| | 2020 | 2019 | 2020 | 2020 | 2019 | 2020 |
| | (n = | (n = | (n = | (n = | (n = | (n = |
| | 32,350) | 5,852) | 4,397) | 32,350) | 5,852) | 4,397) |
| RDS | 7.8 | 6.9 | 7.8 | 0.837 | 0.806 | 0.820 |
| IVH | 20.4 | 26.2 | 35.6 | 0.945 | 0.976 | 0.982 |
| NEC | 39.8 | 44.0 | 45.1 | 0.957 | 0.985 | 0.955 |
| ROP | 40.3 | 42.9 | 41.6 | 0.979 | 0.988 | 0.987 |
| BPD | 58.4 | 81.2 | 66.6 | 0.986 | 0.989 | 0.968 |
| PDA | 13.2 | 15.8 | 15.5 | 0.872 | 0.886 | 0.893 |
| PVL | 64.1 | 54.7 | 67.4 | 0.934 | 0.990 | 0.952 |
| Sepsis | 10.0 | 12.0 | 13.6 | 0.816 | 0.813 | 0.870 |
| Pulmonary hem. | 28.4 | 58.0 | 15.0 | 0.969 | 0.977 | 0.947 |
| CP | 12.2 | 22.2 | | 0.878 | 0.967 | 0.000 |
| Pulmonary HTN | 17.3 | 10.5 | 11.4 | 0.882 | 0.868 | 0.865 |
| Hyperbilirubinemia | 1.4 | 1.3 | 1.3 | 0.648 | 0.615 | 0.611 |
| Death | 42.0 | 22.4 | 43.0 | 0.963 | 0.933 | 0.954 |
| MAS | 1.9 | 1.3 | 2.2 | 0.640 | 0.529 | 0.607 |
| Atelectasis | 29.4 | 16.1 | 16.4 | 0.922 | 0.917 | 0.930 |
| Candidiasis | 3.2 | 1.6 | 4.8 | 0.684 | 0.557 | 0.687 |
| Cardiac failure | 16.7 | 20.1 | 35.1 | 0.940 | 0.963 | 0.985 |
| Cardiovascular | 7.8 | 7.0 | 5.5 | 0.855 | 0.811 | 0.756 |
| Instability | | | | | | |
| Other CNS | 4.1 | 2.4 | 18.4 | 0.776 | 0.738 | 0.715 |
| disorder | | | | | | |
| Neonatal | 6.1 | 4.7 | 4.8 | 0.754 | 0.719 | 0.774 |
| Gastroesophageal | | | | | | |
| Reflux | | | | | | |
| Respiratory failure | 5.7 | 5.2 | 9.0 | 0.766 | 0.755 | 0.810 |
| Polycythemia | 3.3 | 2.8 | 3.9 | 0.727 | 0.756 | 0.848 |
| Seizures | 8.3 | 13.0 | 24.0 | 0.825 | 0.831 | 0.870 |
| Anemia of | 30.9 | 31.3 | 38.3 | 0.986 | 0.986 | 0.987 |
| prematurity | | | | | | |

Note:
RC: random classifier;
RDS: respiratory distress syndrome;
IVH: intraventricular hemorrhage;
NEC: necrotizing enterocolitis;
ROP: retinopathy of prematurity;
BPD: bronchopulmonary dysplasia;
PDA: patent ductus arteriosus;
PVL: periventricular leukomalacia;
CP: cerebral palsy;
MAS: meconium aspiration syndrome;
CNS: central nervous system

TABLE 6
Summary statistics of maternal/newborn characteristics
in the external validation data from UCSF

| | | | Pre-term | | Full-term | |
|---|---|---|---|---|---|---|
| | Overall | | newborns | | newborns | |
| n (%) or mean (±SD) | (n = 12,256) | | (n = 1,856) | | (n = 10,400) | |
| Maternal age at delivery (years) | 32.8 | (±5.3) | 32.9 | (±5.2) | 32.4 | (±6.1) |
| Newborn weight (g) | 3154.8 | (±711.6) | 3354.7 | (467.9) | 2009.1 | (782.1) |
| GA (weeks) | 38.6 | (±3.1) | 39.6 | (±1.2) | 32.8 | (±4.0) |
| Newborn Sex | | | | | | |
| Male | 6192 | (50.5%) | 5292 | (50.9%) | 900 | (48.5%) |
| Female | 5934 | (48.4%) | 5095 | (49.0%) | 839 | (45.2%) |
| Unknown | 130 | (1.1%) | 13 | (0.1%) | 117 | (6.3%) |
| Maternal race | | | | | | |
| American Indian or Alaska Native | 47 | (0.4%) | 41 | (0.4%) | 6 | (0.3%) |
| Asian | 2558 | (20.9%) | 2292 | (22.0%) | 266 | (14.3%) |
| Black or African American | 749 | (6.1%) | 612 | (5.9%) | 137 | (7.4%) |
| Native Hawaiian or Other Pacific | 155 | (1.3%) | 140 | (1.3%) | 15 | (0.8%) |
| Islander | | | | | | |
| Other | 2015 | (16.4%) | 1620 | (15.6%) | 395 | (21.3%) |
| Unknown/Declined | 680 | (5.5%) | 491 | (4.7%) | 189 | (10.2%) |
| White or Caucasian | 6052 | (49.4%) | 5204 | (50.0%) | 848 | (45.7%) |
| Maternal ethnicity | | | | | | |
| Hispanic or Latino | 1618 | (13.2%) | 1289 | (12.4%) | 329 | (17.7%) |
| Not Hispanic or Latino | 9819 | (80.1%) | 8506 | (81.8%) | 1313 | (70.7%) |
| Unknown/Declined | 819 | (6.7%) | 605 | (5.8%) | 214 | (11.5%) |
| Neonatal outcomes | | | | | | |
| RDS | 1092 | (8.9%) | | | | |
| IVH | 199 | (1.6%) | | | | |
| NEC | 30 | (0.2%) | | | | |
| PDA | 431 | (3.5%) | | | | |
| Anemia of prematurity | 287 | (2.3%) | | | | |

TABLE 7
Logistic regression models built using Stanford data to predict RDS, NEC, IVH, PDA and
anemia of prematurity based on the top 10 codes for each outcome plus gestational age

| | | | | |
|---|---|---|---|---|
| Concept code | Concept type | Concept name | β | p |

IVH

| | | | | |
|---|---|---|---|---|
| 2110317 | Procedure | cesarean delivery only including postpartum care | 0.53 | 4.4 × 10⁻⁰³ |
| 1550023 | Drug | insulin lispro | 0.66 | 3.7 × 10⁻⁰⁴ |
| 437334 | Condition | cervical incompetence with antenatal problem | 0.26 | 0.45 |
| 2514404 | Procedure | initial hospital care per day for the evaluation and | 0.17 | 0.31 |
| | | management of a patient which requires these 3 key | | |
| | | components | | |
| 2514421 | Procedure | inpatient consultation for a new or established patient | 0.45 | 0.10 |
| | | which requires these 3 key components | | |
| 1178663 | Drug | indomethacin | 0.20 | 0.41 |
| 432695 | Condition | post-term pregnancy | −0.71 | 0.23 |
| 444098 | Condition | gestation period 40 weeks | −0.73 | 0.21 |
| 19093848 | Drug | magnesium sulfate | 1.06 | 2.0 × 10⁻⁰⁹ |
| 45757175 | Condition | preterm labor in second trimester with preterm | −0.18 | 0.49 |
| | | delivery in second trimester | | |
| | | Gestational age (in days) | −0.04 | 1.3 × 10⁻⁵⁶ |

NEC

| | | | | |
|---|---|---|---|---|
| 4150125 | Condition | persistent pain following procedure | 0.10 | 0.89 |
| 2514422 | Procedure | inpatient consultation for a new or established patient | 0.84 | 0.07 |
| | | which requires these 3 key components | | |
| 2514421 | Procedure | inpatient consultation for a new or established patient | 0.15 | 0.73 |
| | | which requires these 3 key components | | |
| 4150816 | Condition | bicornuate uterus | 1.12 | 0.08 |
| 19037038 | Drug | calcium gluconate | 0.74 | 0.15 |
| 4289303 | Condition | placenta accreta | 0.71 | 0.29 |
| 432695 | Condition | post-term pregnancy | −0.70 | 0.50 |
| 198492 | Condition | second degree perineal laceration | −2.01 | 0.05 |
| 19093848 | Drug | magnesium sulfate | 1.54 | 2.5 × 10⁻⁰⁶ |
| 45757175 | Condition | preterm labor in second trimester with preterm | −0.11 | 0.76 |
| | | delivery in second trimester | | |
| | | Gestational age (in days) | −0.05 | 1.8 × 10⁻³⁰ |

Anemia of prematurity

| | | | | |
|---|---|---|---|---|
| 2211752 | Procedure | ultrasound pregnant uterus real time with image | 0.33 | 0.02 |
| | | documentation fetal and maternal evaluation plus | | |
| | | detailed fetal anatomic examination | | |
| 2213284 | Procedure | level v - surgical pathology gross and microscopic | 1.58 | 4.5 × 10⁻²⁵ |
| | | examination adrenal resection bone | | |
| 2514404 | Procedure | initial hospital care per day for the evaluation and | 0.31 | 0.01 |
| | | management of a patient which requires these 3 key | | |
| | | components | | |
| 1178663 | Drug | indomethacin | 0.19 | 0.38 |
| 442355 | Condition | gestation period 37 weeks | −1.15 | 4.7 × 10⁻⁴ |
| 432695 | Condition | post-term pregnancy | −0.32 | 0.59 |
| 444098 | Condition | gestation period 40 weeks | −0.18 | 0.78 |
| 19093848 | Drug | magnesium sulfate | 1.02 | 1.5 × 10⁻¹⁴ |
| 443871 | Condition | gestation period 38 weeks | −1.61 | 1.5 × 10⁻³ |
| 45757175 | Condition | preterm labor in second trimester with preterm | −1.35 | 3.2 × 10⁻⁶ |
| | | delivery in second trimester | | |
| | | Gestational age (in days) | −0.08 | 2.0 × 10⁻¹⁵⁸ |

RDS

| | | | | |
|---|---|---|---|---|
| 1318853 | Drug | nifedipine | 0.23 | 0.01 |
| 2110317 | Procedure | cesarean delivery only including postpartum care | 0.75 | 1.6 × 10⁻¹⁴ |
| 1178663 | Drug | indomethacin | 0.07 | 0.68 |
| 2514404 | Procedure | initial hospital care per day for the evaluation and | 0.21 | 0.02 |
| | | management of a patient which requires these 3 key | | |
| | | components | | |
| 2211752 | Procedure | ultrasound pregnant uterus real time with image | 0.30 | 4.4 × 10⁻³ |
| | | documentation fetal and maternal evaluation plus | | |
| | | detailed fetal anatomic examination | | |
| 193275 | Condition | third degree perineal tear during delivery - delivered | −13.35 | 0.93 |
| 2514421 | Procedure | inpatient consultation for a new or established patient | 0.31 | 0.06 |
| | | which requires these 3 key components an expanded | | |
| | | problem focused history an expanded problem | | |
| | | focused examination and straightforward medical | | |
| | | decision making counseling and/or coordination of ca | | |
| 4289303 | Condition | placenta accreta | 0.56 | 0.03 |
| 19093848 | Drug | magnesium sulfate | 0.54 | 1.9 × 10⁻¹⁴ |
| 45757175 | Condition | preterm labor in second trimester with preterm | −1.53 | 1.7 × 10⁻⁷ |
| | | delivery in second trimester | | |
| | | Gestational age (in days) | −0.06 | 1.7 × 10⁻²⁹¹ |

PDA

| | | | | |
|---|---|---|---|---|
| 434462 | Condition | ventricular septal defect | 0.34 | 0.21 |
| 1728416 | Drug | penicillin g | −1.66 | 0.02 |
| 4334808 | Procedure | fetal echocardiography | 1.10 | 5.2 × 10⁻⁶ |
| 2313881 | Procedure | doppler echocardiography color flow velocity | 0.21 | 0.61 |
| | | mapping list separately in addition to codes for | | |
| | | echocardiography | | |
| 2722250 | Procedure | echocardiography fetal cardiovascular system real | 0.07 | 0.98 |
| | | time with image documentation 2d with or without m- | | |
| | | mode recording | | |
| 2211763 | Procedure | doppler echocardiography fetal pulsed wave and/or | 0.44 | 0.85 |
| | | continuous wave with spectral display complete | | |
| 312723 | Condition | congenital heart disease | 1.60 | 2.5 × 10⁻³² |
| 2211764 | Procedure | doppler echocardiography fetal pulsed wave and/or | 0.77 | 0.11 |
| | | continuous wave with spectral display follow-up or | | |
| | | repeat study | | |
| 45757175 | Condition | preterm labor in second trimester with preterm | 0.70 | 2.9 × 10⁻³ |
| | | delivery in second trimester | | |
| 2211762 | Procedure | echocardiography fetal cardiovascular system real | 0.87 | 0.07 |
| | | time with image documentation 2d with or without m- | | |
| | | mode recording follow-up or repeat study | | |
| | | Gestational age (in days) | −0.04 | 1.3 × 10⁻¹²⁵ |

TABLE 8
classification accuracy, in terms of AUC, AUPRC and AUPRC compared to a random classifier,
in subgroups identified through subgroup discovery and in the full dataset

| | Subgroup | | | | | | Full dataset | | | | |
|---|---|---|---|---|---|---|---|---|---|---|---|
| | Subgroup | n pos | | | AUPRC | Median [IQR] | n pos | | | AUPRC | Median [IQR] |
| outcome | size | cases | AUPRC | AUC | vs RC | GA (in weeks) | cases | AUPRC | AUC | vs RC | GA (in weeks) |
| RDS | 4079 | 476 | 0.677 | 0.888 | 5.8 | 39.1 (37.7, 39.9) | 2248 | 0.541 | 0.837 | 7.8 | 39.1 (38.1, 40.0) |
| IVH | 4077 | 10 | 0.044 | 0.852 | 18.1 | 39.4 (38.7, 40.1) | 249 | 0.157 | 0.945 | 20.4 | 39.1 (38.1, 40.0) |
| NEC | 4561 | 4 | 0.516 | 0.751 | 588.8 | 39.4 (38.7, 40.1) | 78 | 0.096 | 0.957 | 39.8 | 39.1 (38.1, 40.0) |
| ROP | 3931 | 27 | 0.860 | 0.998 | 125.2 | 39.7 (39.1, 40.4) | 554 | 0.690 | 0.979 | 40.3 | 39.1 (38.1, 40.0) |
| BPD | 3974 | 17 | 0.641 | 0.998 | 149.9 | 39.6 (39.0, 40.3) | 270 | 0.487 | 0.986 | 58.4 | 39.1 (38.1, 40.0) |
| PDA | 4000 | 111 | 0.466 | 0.873 | 16.8 | 39.3 (38.6, 40.0) | 965 | 0.394 | 0.872 | 13.2 | 39.1 (38.1, 40.0) |
| PVL | 4113 | 3 | 0.041 | 0.992 | 56.3 | 39.3 (38.4, 40.0) | 24 | 0.048 | 0.934 | 64.1 | 39.1 (38.1, 40.0) |
| Sepsis | 3893 | 50 | 0.172 | 0.765 | 13.4 | 39.1 (38.3, 40.0) | 579 | 0.179 | 0.816 | 10. | 39.1 (38.1, 40.0) |
| Pulmonary hem. | 5348 | 4 | 0.031 | 0.984 | 41.6 | 39.3 (38.4, 40.0) | 46 | 0.040 | 0.969 | 28.4 | 39.1 (38.1, 40.0) |
| CP | 4134 | 4 | 0.010 | 0.859 | 10.6 | 39.3 (38.4, 40.0) | 66 | 0.025 | 0.878 | 12.2 | 39.1 (38.1, 40.0) |
| Pulmonary HTN | 4385 | 13 | 0.039 | 0.787 | 13.2 | 39.3 (38.4, 39.9) | 154 | 0.082 | 0.882 | 17.3 | 39.1 (38.1, 40.0) |
| Hyperbilirubinemia | 4101 | 2237 | 0.753 | 0.712 | 1.4 | 37.3 (36.1, 39.0) | 15015 | 0.628 | 0.648 | 1.4 | 39.1 (38.1, 40.0) |
| Death | 4672 | 3 | 0.005 | 0.902 | 7.1 | 39.7 (39.0, 40.1) | 230 | 0.298 | 0.963 | 42. | 39.1 (38.1, 40.0) |
| MAS | 3884 | 29 | 0.013 | 0.656 | 1.7 | 39.1 (38.0, 39.9) | 357 | 0.02 | 0.640 | 1.9 | 39.1 (38.1, 40.0) |
| Atelectasis | 4092 | 10 | 0.252 | 0.891 | 103.2 | 39.6 (39.0, 40.3) | 314 | 0.286 | 0.922 | 29. | 39.1 (38.1, 40.0) |
| Candidiasis | 4082 | 18 | 0.071 | 0.695 | 16.1 | 39.4 (38.7, 40.1) | 201 | 0.020 | 0.684 | 3.2 | 39.1 (38.1, 40.0) |
| Cardiac failure | 4088 | 2 | 0.031 | 0.529 | 64.3 | 39.3 (38.7, 40.1) | 42 | 0.022 | 0.940 | 16.7 | 39.1 (38.1, 40.0) |
| Cardiovascular Instability | 3961 | 234 | 0.431 | 0.878 | 7.3 | 39.1 (38.0, 39.9) | 1761 | 0.422 | 0.855 | 7.8 | 39.1 (38.1, 40.0) |
| Other CNS disorder | 4187 | 5 | 0.004 | 0.795 | 3.7 | 39.3 (39.0, 39.9) | 52 | 0.007 | 0.776 | 4.1 | 39.1 (38.1, 40.0) |
| Neonatal Gastroes. Reflux | 3902 | 14 | 0.039 | 0.757 | 10.8 | 39.3 (38.7, 40.0) | 206 | 0.039 | 0.754 | 6.1 | 39.1 (38.1, 40.0) |
| Respiratory failure | 4682 | 101 | 0.150 | 0.792 | 7.0 | 39.1 (38.3, 40.0) | 873 | 0.153 | 0.766 | 5.7 | 39.1 (38.1, 40.0) |
| Polycythemia | 3976 | 15 | 0.012 | 0.726 | 3.1 | 39.3 (38.6, 40.0) | 136 | 0.014 | 0.727 | 3.3 | 39.1 (38.1, 40.0) |
| Seizures | 4018 | 10 | 0.038 | 0.856 | 15.3 | 39.3 (38.7, 40.0) | 116 | 0.030 | 0.825 | 8.3 | 39.1 (38.1, 40.0) |
| Anemia of prematurity | 4693 | 15 | 0.963 | 1.000 | 301.3 | 39.7 (39.3, 40.3) | 750 | 0.717 | 0.986 | 30.9 | 39.1 (38.1, 40.0) |

TABLE 9
AUPRC and AUC (with 95% confidence intervals) of the AI model and the APGAR score at 1 minute to predict the
24 neonatal outcomes in all newborns (n = 32,354). The APGAR score at 1 minute is composed of 5 discrete
subjective scores (each scored 0-2) composed of 1) appearance 2) heart rate 3) grimace 4)activity and 5) respiratory
effort. The APGAR is reflective of an infant's ability to transition to post-natal life with or without the
help of a clinician providing resuscitative interventions. Of note, the APGAR score is a snapshot of subjective
measures and does not necessarily correlate with neonatal outcomes. Neverthless, it is a universal scoring
system with broad application that serves as a measure of post-natal health shortly after birth.

| | AUPRC | | | AUC | | |
|---|---|---|---|---|---|---|
| Outcome | AI model | Apgar | p-value | AI model | Apgar | p-value |
| RDS | 0.541 (0.536, 0.546) | 0.270 (0.265, 0.275) | <0.001 | 0.837 (0.826, 0.848) | 0.761 (0.750, 0.773) | <0.001 |
| IVH | 0.157 (0.153, 0.161) | 0.056 (0.053, 0.058) | <0.001 | 0.945 (0.929, 0.960) | 0.820 (0.790, 0.851) | <0.001 |
| NEC | 0.096 (0.093, 0.099) | 0.016 (0.015, 0.017) | <0.001 | 0.957 (0.928, 0.986) | 0.803 (0.747, 0.860) | <0.001 |
| ROP | 0.690 (0.685, 0.695) | 0.105 (0.102, 0.109) | <0.001 | 0.979 (0.971, 0.987) | 0.822 (0.801, 0.842) | <0.001 |
| BPD | 0.487 (0.482, 0.493) | 0.083 (0.080, 0.086) | <0.001 | 0.986 (0.979, 0.993) | 0.900 (0.881, 0.920) | <0.001 |
| PDA | 0.394 (0.389, 0.400) | 0.087 (0.084, 0.090) | <0.001 | 0.872 (0.857, 0.888) | 0.679 (0.660, 0.698) | <0.001 |
| PVL | 0.048 (0.045, 0.050) | 0.006 (0.006, 0.007) | <0.001 | 0.934 (0.861, 1.000) | 0.862 (0.770, 0.955) | 0.16 |
| Sepsis | 0.179 (0.175, 0.183) | 0.099 (0.096, 0.102) | <0.001 | 0.816 (0.795, 0.837) | 0.766 (0.743, 0.789) | <0.001 |
| Pulmonary hem. | 0.040 (0.038, 0.043) | 0.021 (0.020, 0.023) | <0.001 | 0.969 (0.950, 0.988) | 0.942 (0.915, 0.969) | 0.09 |
| CP | 0.025 (0.023, 0.027) | 0.011 (0.010, 0.013) | <0.001 | 0.878 (0.828, 0.927) | 0.736 (0.663, 0.809) | <0.001 |
| Pulmonary HTN | 0.082 (0.079, 0.085) | 0.030 (0.029, 0.032) | <0.001 | 0.882 (0.849, 0.915) | 0.758 (0.713, 0.803) | <0.001 |
| Hyperbilirubinemia | 0.628 (0.623, 0.633) | 0.492 (0.486, 0.497) | <0.001 | 0.648 (0.642, 0.654) | 0.516 (0.510, 0.522) | <0.001 |
| Death | 0.298 (0.293, 0.303) | 0.167 (0.163, 0.171) | <0.001 | 0.963 (0.951, 0.976) | 0.904 (0.874, 0.933) | <0.001 |
| MAS | 0.021 (0.020, 0.023) | 0.045 (0.042, 0.047) | <0.001 | 0.640 (0.609, 0.671) | 0.702 (0.670, 0.734) | 0.003 |
| Atelectasis | 0.286 (0.281, 0.291) | 0.049 (0.046, 0.051) | <0.001 | 0.922 (0.901, 0.943) | 0.759 (0.728, 0.790) | <0.001 |
| Candidiasis | 0.020 (0.019, 0.022) | 0.009 (0.008, 0.010) | <0.001 | 0.684 (0.644, 0.723) | 0.557 (0.516, 0.599) | <0.001 |
| Cardiac failure | 0.022 (0.020, 0.023) | 0.004 (0.004, 0.005) | <0.001 | 0.940 (0.893, 0.986) | 0.667 (0.567, 0.768) | <0.001 |
| Cardiovascular Instability | 0.422 (0.417, 0.427) | 0.162 (0.158, 0.166) | <0.001 | 0.855 (0.844, 0.867) | 0.690 (0.676, 0.704) | <0.001 |
| Other CNS disorder | 0.007 (0.006, 0.007) | 0.024 (0.022, 0.026) | <0.001 | 0.776 (0.707, 0.845) | 0.855 (0.790, 0.920) | 0.10 |
| Neonatal | 0.039 (0.037, 0.041) | 0.016 (0.014, 0.017) | <0.001 | 0.754 (0.713, 0.795) | 0.646 (0.605, 0.686) | <0.001 |
| Gastroesophageal Reflux | | | | | | |
| Respiratory failure | 0.153 (0.150, 0.157) | 0.137 (0.133, 0.140) | <0.001 | 0.766 (0.747, 0.785) | 0.732 (0.712, 0.752) | 0.02 |
| Polycythemia | 0.014 (0.013, 0.015) | 0.007 (0.006, 0.008) | <0.001 | 0.727 (0.680, 0.774) | 0.605 (0.557, 0.654) | <0.001 |
| Seizures | 0.030 (0.028, 0.032) | 0.025 (0.024, 0.027) | <0.001 | 0.825 (0.779, 0.871) | 0.763 (0.712, 0.814) | 0.04 |
| Anemia of prematurity | 0.717 (0.712, 0.722) | 0.140 (0.136, 0.143) | <0.001 | 0.986 (0.982, 0.990) | 0.813 (0.795, 0.832) | <0.001 |

Note:
RDS: respiratory distress syndrome; IVH: intraventricular hemorrhage; NEC: necrotizing enterocolitis; ROP: retinopathy of prematurity; BPD: bronchopulmonary dysplasia; PDA: patent ductus arteriosus; PVL: periventricular leukomalacia; CP: cerebral palsy; MAS: meconium aspiration syndrome; CNS: central nervous system; p-values obtained using bootstrap.

TABLE 10
AUPRC and AUC (with 95% confidence intervals) of the AI model and the NICHD risk score to predict the 24 neonatal outcomes
in pre-term newborns (n = 3,936). The NICHD model predicts mortality or major morbidities including BPD, NEC, ROP
IVH, white-matter injury and neurodevelopmental impairment for infants born at 22-25 weeks gestation. Of note, this model
was not designed to predict outcomes for infants born outside of 22-25 weeks gestation, or any additional outcomes beyond
the pre-speciefied ones. As such, comparisons between the AI model and the NICHD model must be interpreted with caution.

| | AUPRC | | | AUC | | |
|---|---|---|---|---|---|---|
| Outcome | AI model | NICHD | p-value | AI model | NICHD | p-value |
| RDS | 0.784 (0.770, 0.797) | 0.375 (0.360, 0.391) | <0.001 | 0.840 (0.827, 0.854) | 0.467 (0.448, 0.487) | <0.001 |
| IVH | 0.181 (0.169, 0.194) | 0.104 (0.094, 0.114) | <0.001 | 0.847 (0.825, 0.868) | 0.567 (0.521, 0.614) | <0.001 |
| NEC | 0.106 (0.096, 0.116) | 0.072 (0.064, 0.081) | <0.001 | 0.853 (0.818, 0.887) | 0.595 (0.511, 0.680) | <0.001 |
| ROP | 0.704 (0.689, 0.719) | 0.220 (0.207, 0.234) | <0.001 | 0.919 (0.906, 0.932) | 0.533 (0.505, 0.562) | <0.001 |
| BPD | 0.507 (0.491, 0.523) | 0.177 (0.165, 0.190) | <0.001 | 0.935 (0.924, 0.946) | 0.603 (0.561, 0.645) | <0.001 |
| PDA | 0.441 (0.424, 0.457) | 0.178 (0.166, 0.191) | <0.001 | 0.864 (0.845, 0.883) | 0.526 (0.493, 0.560) | <0.001 |
| PVL | 0.051 (0.044, 0.059) | 0.014 (0.011, 0.019) | <0.001 | 0.885 (0.841, 0.929) | 0.523 (0.326, 0.628) | <0.001 |
| Sepsis | 0.292 (0.277, 0.307) | 0.210 (0.197, 0.224) | <0.001 | 0.812 (0.786, 0.837) | 0.625 (0.588, 0.662) | <0.001 |
| Pulmonary hem. | 0.047 (0.040, 0.054) | 0.071 (0.063, 0.080) | <0.001 | 0.881 (0.847, 0.916) | 0.674 (0.567, 0.782) | <0.001 |
| CP | 0.030 (0.025, 0.036) | 0.012 (0.009, 0.017) | <0.001 | 0.800 (0.734, 0.865) | 0.573 (0.475, 0.671) | <0.001 |
| Pulmonary HTN | 0.093 (0.084, 0.103) | 0.031 (0.026, 0.037) | <0.001 | 0.881 (0.840, 0.921) | 0.532 (0.448, 0.615) | <0.001 |
| Hyperbilirubinemia | 0.866 (0.854, 0.876) | 0.706 (0.690, 0.720) | <0.001 | 0.729 (0.711, 0.747) | 0.490 (0.468, 0.511) | <0.001 |
| Death | 0.319 (0.304, 0.334) | 0.249 (0.235, 0.264) | <0.001 | 0.932 (0.913, 0.951) | 0.616 (0.550, 0.683) | <0.001 |
| MAS | 0.025 (0.021, 0.031) | 0.014 (0.011, 0.019) | <0.001 | 0.670 (0.583, 0.757) | 0.539 (0.452, 0.626) | 0.06 |
| Atelectasis | 0.261 (0.247, 0.275) | 0.087 (0.079, 0.097) | <0.001 | 0.869 (0.842, 0.895) | 0.628 (0.584, 0.672) | <0.001 |
| Candidiasis | 0.046 (0.039, 0.053) | 0.017 (0.014, 0.022) | <0.001 | 0.709 (0.644, 0.775) | 0.516 (0.438, 0.593) | <0.001 |
| Cardiac failure | 0.023 (0.019, 0.029) | 0.007 (0.005, 0.011) | <0.001 | 0.870 (0.794, 0.947) | 0.492 (0.330, 0.655) | <0.001 |
| Cardiovascular Instability | 0.563 (0.547, 0.579) | 0.307 (0.292, 0.322) | <0.001 | 0.775 (0.759, 0.791) | 0.469 (0.448, 0.490) | <0.001 |
| Other CNS disorder | 0.006 (0.004, 0.009) | 0.004 (0.002, 0.006) | 0.28 | 0.640 (0.491, 0.789) | 0.532 (0.340, 0.724) | 0.33 |
| Neonatal | 0.060 (0.053, 0.069) | 0.026 (0.021, 0.032) | <0.001 | 0.740 (0.691, 0.790) | 0.456 (0.476, 0.612) | <0.001 |
| Gastroesophageal Reflux | | | | | | |
| Respiratory failure | 0.216 (0.203, 0.230) | 0.128 (0.117, 0.139) | <0.001 | 0.755 (0.725, 0.784) | 0.519 (0.482, 0.556) | <0.001 |
| Polycythemia | 0.020 (0.015, 0.025) | 0.011 (0.008, 0.015) | 0.19 | 0.593 (0.528, 0.659) | 0.369 (0.554, 0.709) | 0.42 |
| Seizures | 0.044 (0.038, 0.051) | 0.026 (0.021, 0.032) | <0.001 | 0.779 (0.711, 0.848) | 0.505 (0.407, 0.604) | <0.001 |
| Anemia of prematurity | 0.729 (0.715, 0.744) | 0.277 (0.262, 0.292) | <0.001 | 0.905 (0.893, 0.917) | 0.524 (0.499, 0.549) | <0.001 |

Note:
RDS: respiratory distress syndrome; IVH: intraventricular hemorrhage; NEC: necrotizing enterocolitis; ROP: retinopathy of prematurity; BPD: bronchopulmonary dysplasia; PDA: patent ductus arteriosus; PVL: periventricular leukomalacia; CP: cerebral palsy; MAS: meconium aspiration syndrome; CNS: central nervous system; p-values obtained using bootstrap.

What is claimed is:

1. A machine learning model, comprising:
a multitask neural network, wherein the neural network comprises an encoder, a hidden state, and a decoder, wherein the encoder reads an input, wherein the hidden state represents an internal learned representation of the entire input, and wherein the decoder interprets the interprets internal learned representation and reconstructs the input.

2. The machine learning model of claim 1, wherein the input comprises electronic health records (EHR) for an individual.

3. The machine learning model of claim 1, wherein the machine learning model is trained using EHR for a plurality of individuals and a plurality of newborns, wherein each individual in the plurality of individuals has birthed at least one newborn in the plurality of newborns.

4. A method for assessing neonatal risk, comprising:
obtaining or having obtained electronic health records (EHR) for an individual; and
identifying at least one neonatal disorder for a child of the individual based on metabolites in the EHR utilizing a machine learning model comprising deep learning neural network with at least one bottleneck layer.

5. The method of claim 4, wherein the machine learning model determines respiratory support strategies (including ventilator settings) to reduce adverse outcomes.

6. The method of claim 5, wherein the respiratory support strategies includes ventilator settings.

7. The method of claim 4, wherein the model identifies a medication prescribed to a mother than can impact neonatal morbidities.

8. The method of claim 4, wherein the at least one neonatal disorder is selected from bronchopulmonary dysplasia (BPD), intraventricular hemorrhage (IVH), necrotizing enterocolitis (NEC), retinopathy of prematurity (ROP), Bronchopulmonary dysplasia (BPD), intraventricular hemorrhage (IVH), necrotizing enterocolitis (NEC), retinopathy of prematurity (ROP), pulmonary hypertension, pulmonary hemorrhage, jaundice, periventricular leukomalacia (PVL), respiratory distress syndrome (RDS), early onset sepsis, late onset sepsis, patent ductus arteriosus (PDA), cerebral palsy, and neurodevelopmental impairment (NDI).

9. The method of claim 4, wherein the EHR comes from multiple institutions.

10. The method of claim 4, further comprising treating the child for the at least one neonatal disorder.

11. A method for providing intravenous nutrients to a premature baby, comprising:
obtaining or having obtained electronic health records (EHR) for an individual, wherein the EHR comprise details about the individual's health, and the individual is a premature baby; and
selecting a nutrient bag comprising a mix of nutrients to supplement the health of the individual based on a recommendation by a machine learning model.

12. The method of claim 11, wherein the machine learning model comprises a multitask neural network, wherein the neural network comprises an encoder, a hidden state, and a decoder, wherein the encoder reads an input, wherein the hidden state represents an internal learned representation of the input, and wherein the decoder interprets the interprets internal learned representation and reconstructs the input.

13. The method of claim 12, wherein the nutrient bag is one bag of a set of nutrient bags, wherein each bag in the set of nutrient bags is comprised of a composition of nutrients generated by clustering from a bottleneck layer.

14. The method of claim 13, wherein the nutrient bag improves wound healing.

15. The method of claim 13, wherein the nutrient bag improves neurocognitive development.

16. The method of claim 13, wherein the nutrient bag improves respiratory health.

17. The method of claim 13, wherein the nutrient bag improves gastrointestinal health.

18. The method of claim 13, wherein the nutrient bag improves eye health.

19. A method for nutritional support, comprising:
obtaining health information about an individual; and
providing a dietary recommendation for the individual.

20. The method of claim 19, wherein providing a dietary recommendation comprises providing a food recommendation.

21. The method of claim 20, wherein the food recommendation includes at least one baby food recommendation.

22. The method of claim 19, wherein providing a dietary recommendation comprises interfacing with a database of foods and nutritional information.

23. A method for manufacturing intravenous nutritional supplement solutions, comprising:
developing a set of nutritional recipes for intravenous supplementation using a machine learning model;
producing a nutrient bag comprising a recipe from the set of recipes.

24. The method of claim 23, wherein the machine learning model comprises a multitask neural network, wherein the neural network comprises an encoder, a hidden state, and a decoder, wherein the encoder reads an input, wherein the hidden state represents an internal learned representation of the input, and wherein the decoder interprets the interprets internal learned representation and reconstructs the input.

25. The method of claim 23, wherein producing a nutrient bag comprises producing a nutrient bag for each recipe in the set of recipes.

26. The method of claim 23, wherein the set of nutritional recipes comprises at least 5 recipes.

27. The method of claim 23, wherein the set of nutritional recipes comprises 15 recipes.

28. The method of claim 23, wherein the nutrient bag is a sterile IV bag.
